(19) United States
(12) Patent Application Publication
(10) Pub. No.: US 2024/0009249 A1
(43) Pub. Date: Jan. 11, 2024

(54) ADIPOGENIC CELL COMPOSITIONS AND METHODS

(71) Applicant: Flagship Pioneering, Inc., Cambridge, MA (US)

(72) Inventors: Alexandra Sophie DE BOER, Cambridge, MA (US); Le Phuan Lizzie NGO, Cambridge, MA (US); Avak KAHVEJIAN, Cambridge, MA (US); Jordan LEWANDOWSKI, Cambridge, MA (US)

(21) Appl. No.: 18/038,630

(22) PCT Filed: Nov. 24, 2021
(86) PCT No.: PCT/US21/60831

§ 371 Date: May 24, 2023

Related U.S. Application Data
(60) Provisional application No. 63/118,226, filed on Nov. 25, 2020.
(60) Provisional application No. 63/118,237, filed on Nov. 25, 2020.
(60) Provisional application No. 63/118,235, filed on Nov. 25, 2020.
(60) Provisional application No. 63/118,232, filed on Nov. 25, 2020.

(51) Int. Cl. A61K 35/35; C12N 5/077; C12N 5/0775; A61K 35/28; A61K 38/18; A61P 43/00

(52) U.S. Cl. CPC A61K 35/35; C12N 5/0653; C12Y 114/16001; C12N 5/0665; A61K 35/28; A61K 38/1816; A61P 43/00; C12N 2501/2303; C12N 2501/22; C12N 2501/33; C12N 2501/26; C12N 2506/1369

(57) ABSTRACT

Disclosed herein are compositions comprising adipogenic cells that are useful for the treatment, prevention, or amelioration of diseases or disorders.

### CROSS REFERENCE TO RELATED APPLICATION

This application claims the benefit of priority to U.S. Provisional Patent Application Nos. 63/118,226, filed Nov. 25, 2020, 63/118,232, filed Nov. 25, 2020, 63/118,235, filed Nov. 25, 2020, and 63/118,237, filed Nov. 25, 2020, the entire contents of all of which are hereby incorporated by reference.

### INCORPORATION BY REFERENCE OF SEQUENCE LISTING

The present application is being filed with a Sequence Listing in electronic format. The Sequence Listing is provided as a file VL71-001PC_123828-5032_SequenceListing_ST25, created on Nov. 24, 2021, and is 28,672 bytes in size. The information in electronic format of the Sequence Listing is incorporated by reference in its entirety.

### FIELD

This invention relates, in part, to allogenic, non-immunogenic, long-acting compositions comprising adipogenic cells and methods of making and using the same that are useful for the treatment or prevention of a disease or disorder, e.g., in a mammalian subject, such as a human.

### BACKGROUND

Some diseases or disorders are associated with abnormal protein production or complete protein deficiency. For example, hyperphenylalaninemia (HPA) is characterized by elevated levels of the amino acid phenylalanine most commonly due to impaired function of phenylalanine hydroxylase (PAH), the enzyme that catabolizes phenylalanine to tyrosine. Anemia is characterized by reduced red blood cell production caused by the body's inability to produce enough erythropoietin (EPO).

There is presently a paucity of effective treatments for these, and many other, diseases or disorders, and therefore, there remains a need for therapies that are useful for treating these diseases or disorders. Existing cellular therapies have nummerous shortcomings including, poor potency, low levels of expression (e.g., protein, lipid, etc.), cost, short-term engraftment, immunogencity (safety), and poor scalability/manufacturability.

### SUMMARY

In one aspect, the present invention relates to an allogenic, non-immunogenic, long-acting composition including a therapeutically effective amount of a substantially pure adipogenic cells. In some embodiments, the composition is capable of treating, preventing, or ameliorating a disease or disorder in a subject in need thereof. In some embodiments, the composition is capable of treating, preventing, or ameliorating a disease or disorder in the subject when administered in a single administration. In some embodiments, the adipogenic cells are cultured and expanded. In some embodiments, the composition does not result in an inflammatory reaction upon administration. In some embodiments, the composition elicits less than about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% increase in TNF-alpha, IL-2, or IFN-gamma, or any combination thereof, upon administration to a subject. In some embodiments, the composition elicits an increase of about 5%, about 10%, about 15%, about 20%, about 25%, about 30%, about 35%, about 40%, about 45%, about 50%, about 60%, about 70%, about 80%, about 90%, about 100%, about 150%, about 200%, about 250%, about 300%, about 350%, or about 400% or more of IDO, HLA-G, HGF, PGE2, TGFbeta, and IL-6, or any combination thereof, upon administration to a subject. In some embodiments, the adipogenic cells are selected from adipocytes, adipogenic stem cells (ASCs), and CD34⁺ cells. In some embodiments, the adipogenic cells are adipocytes. In some embodiments, the adipocytes are brown/beige adipocytes or white adipocytes. In some embodiments, the adipocytes express and/or secrete one or more of CIDEC, FABP4, PLIN1, LGALS12, ADIPOQ, TUSC5, SLC19A3, PPARG, LEP, CEBPA, or a combination thereof. In some embodiments, the adipocytes are characterized as having one or more, 2 or more, 3 or more, 4 or more, 5 or more, 10 or more, 15 or more, 20 or more, 25 or more, 30 or more, or 35 or more of the following:
a. being post-mitotic;
b. having a lipid content of greater than about 35% (% fresh weight of adipose tissue); optionally greater than about 40%, about 45%, about 50%, about 55%, about 60%, about 65%, about 70%, about 75%, or about 80%;
c. having a fat content in adipose tissue of about 60% to about 95%, optionally 60-94%, about 60% to about 90%, about 60% to about 85%, about 60% to about 80%, about 60% to about 75%, about 60% to about 70%, about 60% to about 65%, about 65% to about 90%, about 70% to about 90%, about 75% to about 90%, about 80% to about 90%, or about 85% to about 90%;
d. having an average fat content of about 80%, optionally about 75 to about 85%;
e. having a water content in adipose tissue of about 5% to about 40%, optionally about 6-36%, about 5% to about 35%, about 5% to about 30%, about 5% to about 25%, about 5% to about 20%, about 5% to about 15%, about 5% to about 10%, about 10% to about 40%, about 15% to about 40%, about 20% to about 40%, about 25% to about 40%, about 30% to about 40%, or about 35% to about 40%);
f. having an average water content of about 15%, optionally about 12.5% to about 17.5%;
g. having a specific gravity of about 1 g/mL, optionally 0.916 g/mL, about 0.5 g/mL, about 0.6 g/mL, about 0.7 g/mL, about 0.8 g/mL, about 0.9 g/mL, about 1.1 g/mL, or about 1.2 g/mL;
h. having a lipid content comprising one or more of stearic acid, oleic acid, linoleic acid, palmitic acid, palmitoleic acid, and myristic acid, a derivative thereof;
i. having a lipid content comprising one or more of free fatty acids, cholesterol, monoglycerides, and diglycerides;
j. having a lipid droplet of a size greater than about 90% of the cell volume, optionally greater than 95% or greater than about 98%, or about 93%, or about 95%, or about 97%, or about 99%;
k. having a lipid droplet comprising at least about 30% to about 99% of the volume of the cell; optionally at least about 40% to about 90%, about 50% to about 90%, about 60% to about 90%, about 70% to about 90% about 80% to about 90%, about 50%, about 60%, about 70%, about 80%, or about 90%;
l. having a surface size of about 20-300 μm in diameter, optionally about 20-300 μm, about 20-200 μm, about 20-100 μm, about 20-500 μm, about 20-30 μm, about 50-300 μm, about 50-200 μm, about 50-100 μm, about 100-300 μm, about 100-200 μm, about 150-300 μm, about 150-200 μm, or about 200-300 μm;
m. having a nucleus volume of about 200-400 μm³, optionally about 200 to about 350 μm³, about 200 to about 300 μm³, about 200 to about 250 μm³, about 250 to about 400 μm³, about 250 to about 350 μm³, about 250 to about 300 μm³, about 300 to about 350 μm³ or about 300 to about 400 μm³;
n. having a total volume of about 4,000-18,000 μm³, optionally about 4000 to about 15000 μm³, about 5000 to about 15000 μm³, about 10000 to about 15000 μm³, about 12500 to about 15000 μm³, about 4000 to about 10000 μm³, about 5000 to about 15000 μm³, about 7500 to about 15000 μm³, about 10000 to about 15000 μm³, about 12500 to about 15000 μm³;
o. having a nucleus to cell ratio of about 1:20-1:90, optionally about 1:20 to about 1:80, about 1:20 to about 1:70, about 1:20 to about 1:60, about 1:20 to about 1:50, about 1:20 to about 1:40, about 1:20 to about 1:30; about 1:30 to about 1:80, about 1:40 to about 1:80, about 1:50 to about 1:80, about 1:60 to about 1:80, or about 1:70 to about 1:80;
p. having a flattened nucleus;
q. having a small cytoplasm of less than about 10% to about 60% of total cell volume, wherein the cytoplasm excludes lipid droplets volume, optionally less than about 20%, less than about 30%, less than about 40%, or less than about 50%;
r. being capable of absorbing and releasing liquids;
s. being buoyant in in water or an aqueous solution, optionally media, or HBSS;
t. having a non-centrally located nucleus;
u. having one or more fat droplets;
v. having a non-spherical cytoplasm;
w. being capable of secreting one or more of adiponectin, leptin, and TNF-alpha;
x. being capable of lipogenesis;
y. being capable of storing triglycerides (TG);
z. being capable of secreting non-esterified fatty acids (NEFA), optionally long chain fatty acids such as oleic acid palmitoleic acid, linoleic acid, arachidonic acid, lauric acid, and stearic acid;
aa. being responsive to hormones;
bb. being responsive to neural input;
cc. having a cell turn-over rate of about 9 years, optionally about 8 to about 10 years;
dd. having an average diameter of about 45 μm, optionally about 47.2 μm, about 40 μm, about; 42.5 μm, about 47.5 μm, or about 50 μm;
ee. a cell population having a diameter distribution wherein about 25% of cells have a diameter of less than about 50 μm; about 40% of cells have a diameter of about 50-69 μm; about 25% of cells have a diameter of about 70-89 μm, and about 10% of cells have a diameter of greater than or equal to about 90 μm;
ff. responsive to atrial natriuretic peptide (ANP);
gg. capable of lipolysis;
hh. expressing receptors that can bind and respond to steroid hormones;
ii. lysed due to phosphatidylcholine;
jj. cell density of about 1 g/ml, optionally about 0.8 g/ml, about 0.9 g/ml, about 1.1 g/ml, about 1.2 g/ml;
kk. greater than about 80% viability, optionally about 85%, about 90%, about 95%, about 97%, about 98%, or about 99%;
ll. greater than about 80% purity, optionally about 85%, about 90%, about 95%, about 97%, about 98%, or about 99%,
mm. adequate potency, optionally amount of Oil Red O eluted greater than about 200 μg/ml; and
nn. negative for microbial contamination.

In some embodiments, the adipocytes are present in the composition at a concentration of about 38,000,000 cells/mL, about 70,000,000 cells/mL to about 3,000,000 cells/mL, or about 40,000,000 cells/mL to about 20,000,000 cells/mL. In some embodiments, the composition comprises about 50,000 to about 6,000,000,000 adipogenic cells, optionally selected from one or more of adipocytes and adipocyte precursor cells (such as adipogenic stem cells (ASCs), and CD34⁺ cells). In some embodiments, the adipogenic cells are ASCs. In some embodiments, the ASCs are present in the composition at a concentration of about 0.1-100 million cells/mL or about 5 million cells/mL. In some embodiments, the composition comprises about 1 million to about 750 million ASCs or about 120 million ASCs. In some embodiments, the composition comprises an ASC concentration of about 250,000 cells/kg to about 4 million cells/kg.

In some embodiments, the ASCs are characterized as having one or more, or one, two, three of the following:
a. viability of about 90% or greater;
b. glucose uptake of about 5 mmol/L to about 10 mmol/L;
c. and lactate production of about 10 mmol/L to about 15 mmol/L.

In some embodiments, the ASCs express elevated levels of one or more of CDw210, CD107b, CD164, and CD253, or any combination thereof, compared to wild type ASCs and/or unenriched ASCs. In some embodiments, the ASCs express reduced levels of one or more of CD266, CD151, CD49c, and CD9, or any combination thereof compared to wild type ASCs and/or unenriched ASCs. In some embodiments, the ASCs express elevated levels of one or more of CD361, CD120b, CD164, and CD213A1, any combination thereof compared to wild type ASCs and/or unenriched ASCs. In some embodiments, the ASCs express reduced levels of one or more of CD266, CD167, CD325, and CD115, or any combination thereof compared to wild type ASCs and/or unenriched ASCs. In some embodiments, the ASCs express elevated levels of one or more of CDw210b, CD340 and CDw293, or any combination thereof compared to wild type ASCs and/or unenriched ASCs. In some embodiments, the ASCs express reduced levels of one or more of CD151, CD10, CD26, and CD142, or any combination thereof compared to wild type ASCs and/or unenriched ASCs. In some embodiments, less than about 5% of ASCs express one or more of the surface markers HLAII, CDI Ib, CDI Ic, CD14, CD45, CD31, CD34, CD80 and CD86. In some embodiments, at least about 90% or at least about 95% of the ASCs express one or more of the surface markers HLA I, CD29, CD44, CD59, CD73, CD90, and CD105. In some embodiments, the ASCs express elevated levels of CD10 compared to wild type ASCs and/or unenriched ASCs. In some embodiments, at least about 90% or at least about 95% of the ASCs express CD10 compared to wild type ASCs and/or unenriched ASCs. In some embodiments, the ASCs comprise a population of ASCs selectively enriched for CD10 and/or unenriched ASCs. In some embodiments, the adipogenic cells are white adipocytes obtainable by CD10-enriched ASCs and/or unenriched ASCs. In some embodiments, the adipogenic cells are CD34⁺ cells. In some embodiments, the adipogenic cells are mammalian adipogenic cells. In some embodiments, the adipogenic cells are selected from human adipogenic cells or adipogenic cells suitable for use in a human subject. In some embodiments, the adipogenic cells, upon administration to a subject, provide a therapeutically effective amount of adipocytes. In some embodiments, the adipogenic cells, upon administration to a subject, provide a therapeutically effective amount of one or more proteins and/or other molecules, including, but not limited to, erythropoietin (EPO); adipsin; phenylalanine hydroxylase (PAH); adiponectin; PEX5; ATP:cob(1)alamin adenosyl transferase (MMAB); 14-3-3 protein epsilon; 2-oxoisovalerate dehydrogenase subunit alpha, mitochondrial, BCKDHA; 2-Oxoisovalerate dehydrogenase subunit beta, mitochondrial, BCKDHB; 3-Hydroxyisobutyrate dehydrogenase (HIBADH); 3-Hydroxyisobutyryl-CoA deacylase (HIBCH); 3-Methylcrotonyl CoA carboxylase, MCCC1; 3-Methylcrotonyl CoA carboxylase, MCCC2; 4-Aminobutyrate-α-ketoglutarate aminotransferase (ABAT); 5-nucleotidase; 6-phosphogluconate dehydrogenase, decarboxylating; medium-chain acyl-CoA dehydrogenase, MCAD; short-chain acyl-CoA dehydrogenase, SCAD; very long-chain acyl-CoA dehydrogenase, VLCAD; Acetyl-CoA thiolase (acetyl-coenzyme A acetyltransferase), ACAT1; Acid ceramidase; Adenine phosphoribosyltransferase, APRT; Adenosine deaminase; Adipocyte enhancer-binding protein 1; Agrin; Aldehyde oxidase; Aldo-keto reductase family 1 member C2; Alkaline phosphatase, tissue-nonspecific isozyme; Alkyldihydroxyacetonephosphate synthase, AGPS; Alpha-2-macroglobulin; Alpha-enolase; Alpha-fetoprotein; Alpha-L-iduronidase, Alpha-N-acetylglucosaminidase; Alpha-N-acetylglucosaminidase 82 kDa form; Alpha-N-acetylglucosaminidase 77 kDa form; Aminoacylase-1; Angiotensinogen; Angiotensin-1; Angiotensin-2; Angiotensin-3; Angiotensin-4; Angiotensin 1-9; Angiotensin 1-7; Angiotensin 1-5; Angiotensin 1-4; Annexin A5; Adaptor Related Protein Complex 3 Subunit Beta 1, AP3B1; Apolipoprotein E; Argininosuccinate lyase, ASL; Argininosuccinate synthase; Argininosuccinic acid synthetase, ASS; Arylsulfatase A; Arylsulfatase A component B; Arylsulfatase A component C; Arylsulfatase B; aspartylglucosaminidase; ATP-binding cassette transporter, ABCD1; ATP-dependent RNA helicase, DDX3X; Endorepellin; Beta-2-microglobulin; Beta-galactosidase; Beta-hexosaminidase subunit alpha, HEXA; Beta-hexosaminidase subunit beta, HEXB; Bifunctional purine biosynthesis protein, PURH; Biglycan; Biotinidase; Biotinidase; Bone morphogenetic protein 1; Branching enzyme, GBE1; Calmodulin; Calreticulin; cAMP-dependent protein kinase catalytic subunit gamma; Cartilage oligomeric matrix protein; Cartilage-associated protein; Catalase; Catalase, CAT; Cathepsin A; Cathepsin B; Cathepsin D; Cathepsin F; Cathepsin K; Citrin, SLC25A13; Collagen alpha-1(1) chain; Collagen alpha-1(III) chain; Collagen alpha-1(IV) chain; Arresten; Collagen alpha-1(V) chain, Collagen alpha-1(XI) chain, Collagen alpha-1(XVIII) chain; Endostatin, Collagen alpha-2(I) chain; Collagen alpha-2(IV) chain; Canstatin; Collagen alpha-2(V) chain; Collagen alpha-2(VI) chain; Collagen alpha-3(VI) chain; Complement C1r subcomponent; Complement C1s subcomponent; Complement C3; Complement C4 beta chain; Complement factor D; Carnitine palmitoyltransferase 1A, CPT1A; Cystathionine β-synthase, CBS; Cystatin-C; Cystinosin, CTNS; Cytochrome c; Cytokine receptor-like factor 1; Cytoplasmic acetoacetyl-CoA thiolase, ACAT2; D-bifuncitonal enzyme, HSD17B4; Decorin; Dihydrolipoyl dehydrogenase, mitochondrial; Dihydroxyacetonephosphate acyltransferase, GNPAT; Dipeptidyl peptidase 1; Cathepsin C; EGF-containing fibulin-like extracellular matrix protein 1; EGF-containing fibulin-like extracellular matrix protein 2; Elastin; Elongation factor 2; Electron Transfer Flavoprotein Subunit Alpha, ETFA; Electron Transfer Flavoprotein Subunit Beta, ETFB; Electron transfer flavoprotein dehydrogenase, ETFDH; Extracellular matrix protein 1; Fibrillin-1; Fibrillin-2; Fibronectin; Fibulin-1; Fibulin-5; Formyl-Glycin generating enzyme, SUMF1; Fructose 1,6-biphosphatase, FBP1; Fumarylacetoacetase; Fumarylacetoacetate hydrolase domain-containing protein 2A, FAHD2A; Galactocerebrosidase; Galactokinase 1; Galactose-1-phosphate uridyl transferase, GALT; Ganglioside GM2 activator; Ganglioside GM2 activator isoform short; Gelsolin; GIcNAc phosphotransferase, GNPTA; Glucose-6-phosphate 1-dehydrogenase; Glucose-6-phosphate isomerase; Glucose-6-phosphate translocase, G6PT1; Glutaryl CoA dehydrogenase, GCDH; Glutathione peroxidase 3; Glutathione synthetase; Glycerol kinase; Glycerol-3-phosphate dehydrogenase [NAD(+)], cytoplasmic; Glycine cleavage enzyme system, AMT; Glycine cleavage enzyme system, GCSH; Glycogen debranching enzyme; 4-alpha-glucanotransferase; Amylo-alpha-1,6-glucosidase; Glycogen phosphorylase, liver form; Glypican-1; Glypican-6; Hydroxyacyl-CoA Dehydrogenase Trifunctional Multienzyme Complex Subunit Alpha, HADHA; Haptoglobin; Heparan N-sulfatase, N-sulfoglucosamine sulfohydrolase, SGSH; Heparan-alpha-glucosaminide N-acetyltransferase, HGSNAT; Hormone-sensitive lipase; Hydroxyacyl-coenzyme A dehydrogenase, mitochondrial; Hyperactivity of glutamate dehydrogenase, GLUD1; Hypoxanthine-guanine phosphoribosyltransferase, HPRT; Iduronate-2-sulfatase, IDS; Insulin-like growth factor-binding protein 7; Interstitial collagenase; Isovaleryl-CoA dehydrogenase; Keratin, type II cytoskeletal 1; Keratin, type II cytoskeletal 6B; L-lactate dehydrogenase A chain; L-lactate dehydrogenase B chain; Lactoylglutathione lyase; Laminin subunit alpha-2; Laminin subunit alpha-4; Laminin subunit beta-1; Laminin subunit beta-2; Laminin subunit gamma-1; Leptin; Lipoamide acyltransferase component of branched-chain alpha-keto acid dehydrogenase complex, mitochondrial, DBT; Lipoprotein lipase; Liver and muscle phosphorylase kinase, PHKB; Liver phosphorylase kinase, PHKG2; Lysosomal acid lipase/cholesteryl ester hydrolase; Lysosomal alpha-glucosidase; Lysosomal alpha-mannosidase; Lysosomal protective protein; CLN6 Transmembrane ER Protein, CLN6; CLN8 Transmembrane ER And ERGIC Protein, CLN8; Lysosomal transmembrane CLN3 protein, CLN3; Lysosomal transmembrane CLN5 protein, CLN5; Lysosome-associated membrane glycoprotein 2; Lysosomal trafficking regulator, LYST; Malonyl-CoA decarboxylase, MLYCD; Matrilin-3; Matrix Gla protein; Melanophilin, MLPH; Methionine synthase reductase, MTRR; Methylene tetrahydrofolate homocysteine methyltransferase, MTR; Methylenetetrahydrofolate reductase, MTHFR; Methylmalonic semialdehyde dehydrogenase, ALDH6A1; Methylmalonyl-CoA mutase; Mevalonate kinase; Mitochondrial branched-chain aminotransferase 2, BCAT2; Mitochondrial ornithine translocase, SLC25A15; Methylmalonic aciduria type A, MMAA; Molybdopterin synthase, Gephyrin, MOCS1A; Mucolipin-1, MCOLN1; Muscle phosphorylase kinase, PHKA1; Myosin Va, MYO5A; Myosin light chain 4; N-Acetylgalactosamine-6 Sulfatase, GALNS; N-acetylglucosamine-6-sulfatase; Nicotinamide N-methyltransferase; NPC intracellular cholesterol transporter 1, NPC1; Palmitoyl-protein thioesterase-1, PPT1; Palmitoyl-protein thioesterase, PPT2; Pentraxin-related protein, PTX3; Peptidyl-prolyl cis-trans isomerase, FKBP10; Peroxidasin homolog; Peroxin-1, 2, 3, 5, 6, 7, 10, 12, 13, 14, 26, Phosphoacetylglucosamine mutase; Phosphoglucomutase-1; Phosphoglycerate kinase 1; Phosphoglycerate mutase 1; Pigment epithelium-derived factor, PEDF; Plasma alpha-L-fucosidase; Plasma membrane carnitine transport, OCTN2; Plasma protease C1 inhibitor; Plasminogen activator inhibitor 1; Procollagen-lysine,2-oxoglutarate 5-dioxygenase 1; Propionyl-CoA carboxylase; Prosaposin; Proteoglycan 4; Proteoglycan 4 C-terminal part; Pyruvate carboxylase; Pyruvate dehydrogenase complex, DLAT; Pyruvate dehydrogenase complex, PDHB; Pyruvate dehydrogenase complex, PDHX; Pyruvate dehydrogenase complex, PDP1; Ras-related protein Rab-27A, RAB27A; Retinol-binding protein 4; Ribonuclease T2; Semaphorin-7A; Sepiapterin reductase; Serine protease, HTRA1; Serotransferrin; Serpin B6; Serum amyloid A-1 protein; Short branched-chain acyl-CoA dehydrogenase, ACADSB; Sialic acid synthase; Sialidase-1; Sialin (sialic acid transport), SLC17A5; Solute Carrier Family 22 Member 5, SLC22A5; SPARC-related modular calcium-binding protein 2; Spectrin alpha chain, non-erythrocytic 1; Sphingomyelin phosphodiesterase, SMPD1; Succinyl-CoA 3-oxoacid-CoA transferase, OXCT1; Sushi repeat-containing protein, SRPX2; Tafazzin; Tenascin; Thrombospondin-2; Transforming growth factor-beta-induced protein ig-h3; Transitional endoplasmic reticulum ATPase; Triosephosphate isomerase; Tripeptidyl-peptidase 1; Tumor necrosis factor receptor superfamily member 11B; Vascular endothelial growth factor C; Versican core protein; Vimentin; Vitamin K-dependent protein S; X-linked phosphorylase kinase, PHKA2; Xaa-Pro dipeptidase; α-Fucosidase, FUCA1; α-Galactosidase A, GLA; α-N-Acetylglucosaminidase, NAGA; β-Glucocerebrosidase (aka Glucosylceramidase); GBA, β-glucuronidase, GUSB; β-mannosidasen; VEGFA; VEGF165; FGF2; FGF4; PDGF-BB (platelet-derived growth factor); Ang1 (angiopoiten 1), TGFβ (transforming growth factor); LPA-producing enzyme (AXT); and phthalimide neovascularization factor (PNF1).

In some embodiments, the adipogenic cells comprise a heterologous nucleic acid. In some embodiments, the heterologous nucleic acid comprises an adipocyte-specific promoter, optionally an adiponectin promoter or an aP2/FABP4 promoter optionally comprising a minimal proximal promoter sequence, and optionally further comprises one or more of a distal enhancer sequence and additional transcription factor binding sites, optionally C/EBPa binding sites. In some embodiments, the adipocyte specific promoter is an adiponectin promoter, optionally C/EBPa binding sites. In some embodiments, the adipocyte specific promoter is in operative association with a therapeutic protein.

In one aspect, the present invention relates to an autologous, non-immunogenic, long-acting composition comprising a therapeutically effective amount of substantially pure adipogenic cells, wherein the adipogenic cells comprise one or more heterologous nucleic acid.

In some embodiments, the adipogenic cells are at least about 60%, about 65%, about 70%, about 75%, about 80%, about 85%, about 90%, about 91%, about 92%, about 93%, about 94%, about 95%, about 96%, about 97%, about 98%, or about 99% or more viable. In some embodiments, the composition is substantially free of one or more bacteria, virus, fungus, and pyrogen. In some embodiments, the composition comprises a pharmaceutically acceptable carrier, diluent, excipient, or vehicle. In some embodiments, the diluent comprises one or more of saline, phosphate buffered saline, Dulbecco's Modified Eagle Medium DMEM, alpha modified Minimal Essential Medium (alpha.MEM), Roswell Park Memorial Institute Media 1640 (RPMI Media 1640), HBSS, human albumin, and Ringer's solution and the like, or any combination thereof. In some embodiments, the composition comprises a therapeutically effective amount of one or more of heparin, FBS, human albumin, bFGF, PPAR-y agonists, insulin, and a Rho kinase inhibitor, or any combination thereof. In some embodiments, the composition comprises a scaffold. In some embodiments, the scaffold comprises biodegradable biomaterials, optionally natural biomaterials such as collagen, various proteoglycans, alginate-based substrates and chitosan. In some embodiments, the scaffold comprises synthetic biomaterials, optionally synthetic polymer-based materials. In some embodiments, the scaffold comprises one or more of a hydrogel, a matrigel, alginates, collagens, chitosans, PGAs, PLAs, and PGA/PLA copolymers, silk, acellular/de-cellularized scaffolds, optionally from cadavers or non-human animals, biodegradable biomaterials, optionally collagen, proteoglycans, alginate-based substrates, chitosan, or any combination thereof. In some embodiments, the composition further comprises a therapeutically effective amount of one or more additional therapeutic agents. In some embodiments, the additional therapeutic agent is one or more of an analgesic and an anti-infective agent. In some embodiments, the composition is formulated for administration by a route selected from subcutaneous, intradermal, intramuscular, intracranial, intraocular, intravenous, and fat pad. In some embodiments, the adipogenic cells persist up to 1 day, up to 2 days, up to 3 days, up to 4 days, up to 5 days, up to 6 days, up to 7 days, up to 2 weeks, up to 3 weeks, up to 1 month, up to 2 months, up to 3 months, up to 4 months, up to 5 months, up to 6 months, up to 7 months, up to 8 months, up to 9 months, up to 10 months, up to 11 months, up to 1 year, or up to 2 years post engraftment, or more. In some embodiments, the adipogenic cells secrete one or more proteins and/or other molecules up to 1 day, up to 2 days, up to 3 days, up to 4 days, up to 5 days, up to 6 days, up to 7 days, up to 2 weeks, up to 3 weeks, up to 1 month, up to 2 months, up to 3 months, up to 4 months, up to 5 months, up to 6 months, up to 7 months, up to 8 months, up to 9 months, up to 10 months, up to 11 months, up to 1 year, or up to 2 years post engraftment, or more.

In one aspect, the present invention relates to a syringe comprising a composition of the disclosure. In some embodiments, the syringe is prefilled, optionally with a volume of less than about 3 mL or about 2 mL or less. In some embodiments, the composition is stable for at least 12, 24, 36, or 48 hours, and exhibits less than about 35%, about 30%, about 25%, about 20%, about 19%, about 18%, about 17%, about 16%, about 15%, about 14%, about 13%, about 12%, about 11%, about 10%, about 9%, about 8%, about 7%, about 6%, or about 5% loss of cell viability when stored at −80° C., −20° C., 4° C., or 21° C.

In one aspect, the present invention relates to a method for treating, preventing, or ameliorating a disease or disorder in a subject in need thereof. In some embodiments, the method includes administering a composition of the disclosure to the subject, optionally via a syringe of the disclosure.

In some embodiments, the subject is a mammal, optionally a primate. In some embodiments, subject is a human, optionally an adult human, a child, or an infant. In some embodiments, the composition is administered in a single administration, optionally at a single site or multiple sites. In some embodiments, the composition is administered in multiple administrations, optionally at a single site or multiple sites). In some embodiments, the composition is administered by subcutaneous injection. In some embodiments, a combined remission or clinical remission of the disease or disorder is achieved within 24, 18, 12, 8, or 6 weeks from administration. In some embodiments, the subject has, is suspected of having, or is suspected of having an elevated risk for a disease or disorder selected from Lysosomal storage disorders, Metabolic disorders, Complement deficiencies, Adipocyte disorders, Endocrine disorders, Vascular diseases, Branched-chain amino acid metabolism disorders, Connective tissue disorders, Fatty acid transport and mitochrondrial oxidation disorders, Genetic dyslipidemias, Hematological disorders, Phenylalanine and tyrosine metabolism disorders, Purine metabolism disorders, Urea cycle disorders, Beta-amino acid and gamma-amino acid disorders, Ketone metabolism disorders, Galactosemia, Glycerol Metabolism Disorders, Glycine Metabolism Disorders, Lysine Metabolism Disorders, Methionine and Sulfur Metabolism Disorders, and Peroxisome biogenesis and very long chain fatty acid metabolism disorders. In some embodiments, the disease or disorder is selected from Wolman disease, Obesity, C3 deficiency, Familial lipodystrophy, Cachexia, Hereditary angioedema, Propionic acidemia Type 1, Ehlers-Danlos syndrome, long-chain 3-hydroxy acyl-CoA dehydrogenase deficiency, Familial LPL deficiency, Protein S deficiency, Tyrosinemia type I, Adenine phosphoribosyltransferase deficiency, Citrullinemia type I, Methylmalonic semialdehyde dehydrogenase deficiency, Succinyl-CoA 3-oxoacid-CoA transferase deficiency, Galactose-1-phosphate uridyl transferase deficiency, Glycerol kinase deficiency, Nonketotic hyperglycinemia, Glutaric acidemia type I, Molybdenum cofactor defect, and Zellweger syndrome. In some embodiments, the composition comprises adipogenic cells that are not transformed. In some embodiments, the subject has, is suspected of having, or is suspected of having elevated risk a disease or disorder selected from Lysosomal storage disorders, Metabolic disorders, Hematological disorders, Bone and connective tissue disorders, Endocrine disorders, Inflammatory disorders, Monogenic disorders, Cancer, Cardiovascular disorders, Branched-chain amino acid metabolism disorders, Fatty acid transport and mitochrondrial oxidation disorders, Genetic dyslipidemias, Phenylalanine and tyrosine metabolism disorders, Purine metabolism disorders, Urea cycle disorders, Ketone metabolism disorders, Glycine Metabolism Disorders, Lysine Metabolism Disorders, Methionine and Sulfur Metabolism Disorders, Peroxisome biogenesis and very long chain fatty acid metabolism disorders, and other protein deficiency disorders. In some embodiments, the disease or disorder is selected from Cystinosis, T2D, Hemophilia A or B, Stickler syndrome, Osteoporosis, Rheumatoid Arthritis, A1AT deficiency, Breast cancer, Atherosclerosis, Isobutyryl-CoA dehydrogenase deficiency, carnitine-acylcarnitine translocase deficiency, Sitosterolemia, Phenylketonuria, Hereditary xanthinuria, Ornithine-transcarbamoylase deficiency, 3-Hydroxy-3-methylglutaryl-CoA synthase deficiency, Nonketotic hyperglycinemia, Hyperlysinemia, Homocystinuria, Refsum disease, and growth failure in children with kidney disease. In some embodiments, the composition comprises adipogenic cells that are transformed, optionally comprising a heterologous nucleic acid comprising a therapeutic transgene. In some embodiments, the adipogenic cells comprise one or more of a genes, or genes associated with cystinosin, GLP-1, Factor VIII, Factor IX, COL2A1, Parathyroid hormone (1-84), alkaline phosphatase, alpha-1 antitrypsin, Trastuzumab, Apolipoprotein A1, Isobutyryl-CoA dehydrogenase, SLC25A20, ATP-binding cassette sub-family G member 5, ABCG5, Phenylalanine hydroxylase, Xanthine dehydrogenase, Ornithine-transcarbamoylase, 3-Hydroxy-3-methylglutaryl-CoA synthase, Glycine cleavage system P protein, Lysine:α-ketoglutarate reductase, Cystathionine β-synthase, Phytanoyl-CoA hydroxylase, and human growth hormone (somatotropin), wherein the gene is in operative association with an adipocyte-specific promoter. In some embodiments, the adipogenic cells are CD34⁺ cells and the disease or disorder is selected from Wolman disease, Obesity, C3 deficiency, Familial lipodystrophy, Cachexia, Hereditary angioedema, Propionic acidemia Type 1, Ehlers-Danlos syndrome, long-chain 3-hydroxy acyl-CoA dehydrogenase deficiency, Familial LPL deficiency, Protein S deficiency, Tyrosinemia type I, Adenine phosphoribosyltransferase deficiency, Citrullinemia type I, Methylmalonic semialdehyde dehydrogenase deficiency, Succinyl-CoA 3-oxoacid-CoA transferase deficiency, Galactose-1-phosphate uridyl transferase deficiency, Glycerol kinase deficiency, Nonketotic hyperglycinemia, Glutaric acidemia type I, Molybdenum cofactor defect, and Zellweger syndrome.

In one aspect, the present invention relates to the use of a composition of the disclosure in treating, preventing, or ameliorating a disease or disorder.

In one aspect, the present invention relates to the use of a composition of the disclosure in the manufacture of a medicament for treating, preventing, or ameliorating a disease or disorder.

In one aspect, the present invention relates to the use of process for in vivo electroporation of adipogenic cells. In some embodiments, the method includes injecting the adipogenic cells into adipose tissue of a subject, placing the adipose tissue between a first plate electrode and a second plate electrode, and passing a current from the first plate electrode through the adipose tissue to the second plate electrode.

In one aspect, the present invention relates to an allogenic, non-immunogenic, long-acting composition comprising a therapeutically effective amount of substantially pure adipogenic cells, wherein the adipogenic cells comprise a heterologous nucleic acid. In some embodiments, the adipogenic cells express elevated levels of CD10 compared to wild type adipogenic cells and/or unenriched adipogenic cells. In some embodiments, at least about 90% or at least about 95% of the adipogenic cells express CD10. In some embodiments, wherein the adipogenic cells are obtainable from CD10-enriched ASCs. In some embodiments, the adipogenic cells are white adipocytes obtainable from ASCs that expresses elevated levels of CD10 compared to wild type ASCs and/or unenriched ASCs. In some embodiments, the heterologous nucleic acid comprises an adipocyte-specific promoter.

In one aspect, the present invention relates to an allogenic, non-immunogenic, long-acting composition comprising a therapeutically effective amount of a substantially pure adipogenic cells, wherein the adipogenic cells are obtainable from ASCs that expresses elevated levels of CD10 compared to wild type ASCs and/or unenriched ASCs. In some embodiments, the adipogenic cells comprise a heterologous nucleic acid. In some embodiments, the heterologous nucleic acid comprises an adipocyte-specific promoter. In some embodiments, at least about 90% or at least about 95% of the adipogenic cells express CD10. In some embodiments, the adipogenic cells are white adipocytes.

### BRIEF DESCRIPTION OF THE DRAWINGS

FIGS. 1A-1B depict representative images of human ASCs (FIG. 1A) and murine ASCs (FIG. 1B) in culture after 2 passages. ASCs were isolated from adipose tissues using either the enzymatic digestion method or the explant culture method described in Example 1. Isolated ASCs were expanded in culture, and their images were captured using transmitted light and 20× in a M5000 EVOS imaging system.

FIGS. 2A-2B depict experimental data demonstrating the characterization of surface markers of ASCs isolated from human adipose tissues and expanded in culture. The cells were stained with fluorophore-conjugated antibodies against CD29, CD73, CD90, CD105, CD31, CD45, and CD34 and analyzed with flow cytometry. FIG. 2A depicts experimental data representative of gating strategy for stained ASCs. Most of the ASCs (>97%) are positive for CD73, CD105, and CD90 and negative for CD34, CD45, and CD31. FIG. 2B depicts distributions of fluorescence intensity for different cell surface markers in unstained vs stained ASCs. Stained ASCs display a homogenous normal distribution for both positive and negative markers. Unstained cells are represented as dash lines and stained cells as solid lines.

FIGS. 3A-3B depict experimental data demonstrating the characterization of adipocytes derived from ASC differentiation in culture. FIG. 3A depicts Oil Red O staining of ASCs and differentiated ASCs. The cells were fixed with 10% formaldehyde and stained with Oil Red O solution. The images were captured using RBG transmitted light with a 20× objection in an M5000 EVOS imaging system. Oil Red O binds to neutral lipids and stains lipid droplets dark red. In the differentiated culture, >80% of the cells are round in shape and contain a large number of lipid droplets, shown as dark spheres in the right image. These are differentiated adipocytes. FIG. 3B depicts gene expression levels of adipocyte-specific genes in undifferentiated ASCs and differentiated ASCs. The gene expression levels for adiponectin, PPAR γ, leptin, CIDEC, and FABP4 were quantified using RT-PCR and normalized to actin. All expression levels were then normalized to control (undifferentiated ASCs). All adipocyte-specific genes are significantly upregulated in the differentiated ASCs compared to control.

FIG. 4 depicts a human adiponectin promoter mapping. Minimal elements of human adiponectin promoter include the adiponectin distal enhancer (−2667 to −2507 bp) and the adiponectin proximal promoter region (−540 to +77 bp). The distal enhancer contains 2 binding sites for the transcription factor C/EBPα. The distal enhancer and proximal promoter region together are both necessary and sufficient for transcriptional activation of the human adiponectin promoter.

FIG. 5 depicts aP2/FABP4 promoter mapping. Minimal elements of ap2 promoter include the aP2 distal enhancer (−5.4 kb to −4.9 kb) and the ap2 proximal promoter region (−63 to +21 bp). The distal enhancer and proximal promoter region together are necessary and sufficient for transcriptional activation of the aP2 promoter.

FIGS. 6A-6B depict experimental data showing long-term engraftment of adipocytes derived from transplanted human ASCs in mice (in vivo). Human adipsin (FIG. 6A) and FABP4 (FIG. 6B) were detected at day 117 post-transplant in the dorsal flank.

FIG. 7 depicts experimental data demonstrating in vivo secretion of gaussia luciferase by adipocytes derived from transplanted genetically modified adipogenic cells and long-term engraftment of adipocytes derived from transplanted human ASCs in mice (in vivo). Donor-derived adipocytes expressed GLuc for at least 84 days in recipient mice.

FIG. 8 depicts experimental data demonstrating transplantation of adipocytes and in vivo secretion of adipsin. Human adipsin level was detected in plasma up to 126 days post transplantation.

FIGS. 9A-9F depicts experimental data demonstrating non immunogeneic adipogenic cells (in vivo). No innate immune response was detected at 5 hours and day 5 post transplantation in hASCs and culture-derived hAdipocytes. Levels of TNFα (FIG. 9A), IFNγ (FIG. 9B), IL1β (FIG. 9C), IL6 (FIG. 9D), IL10 (FIG. 9E), and IL2 (FIG. 9F) were measured.

FIG. 10 depicts experimental data demonstrating non immunogeneic adipogenic cells (in vitro).

FIGS. 11A-11B depict images demonstrating long-term engraftment of xenografted human adipose cells in immune competent mice (in vivo) at days 92 (FIG. 11A) and 151 post implantation (FIG. 11B).

FIGS. 12A-12B depict experimental data demonstrating localized biodistribution of transplanted adipocytes. FIG. 12A depicts experimental data demonstrating that luciferase analyzed from day 3-day 98 post transplantation was detected at all timepoints in mice measured in transplant-naïve mice and mice transplanted with adipocytes. FIG. 12B depicts images of luciferase activity in mice measured at day 14 and day 98.

FIGS. 13A-13C depict experimental data demonstrating the increased adipogenic potentiation of CD10+ cells. CD10+ selected ASC populations produced adipocytes that secrete significantly higher levels of adiponectin compared to the control and CD10−. FIG. 13A depicts a schematic for a non-limiting method of culturing and differentiating adipose stem cells into adipocytes.

FIG. 13B depicts images demonstrating ASCs at day 7 post induction. FIG. 13C depicts experimental data demonstrating adiponectin protein in media at day 7 for control, CD10+ and CD10-adipocytes.

FIG. 14A-14B depict experimental data demonstrating the ability to generate and characterize adipocytes that secrete a mammalian serum protein. FIG. 14A depicts a schematic for a non-limiting method of preparing adipocytes that secrete EPO. FIG. 14B depicts experimental data demonstrating adipocyte specific EPO expression (in vitro). Levels of hEPO in hEPO engineered cells and unengineered control cells were detected.

FIG. 15A depicts a schematic for a non-limiting method of preparing adipocytes that secrete gaussia luciferase (GLuc). FIG. 15B depicts experimental data demonstrating adipocyte specific gLUC expression in vitro). Engineered ASCs secreted more GLuc as they were further differentiated into adipocytes.

FIGS. 16A-16D depict experimental data demonstrating the therapeutic effects in mice by transplanting ASCs and adipogenic cells genetically modified to secrete EPO. Levels in the mice transplanted with hEPO expressing ASCs and adipocytes rose above the levels in the control mice and remained higher for 30+ days. FIGS. 16A and 16C depict experimental data demonstrating EPO levels in plasma. FIGS. 16B and 16D depict experimental data demonstrating reticulocyte counts.

FIGS. 17A-17D depict experimental data demonstrating that allogeneic ASCs of the disclosure are non-immunogenic as demonstrated by a lack of cell death in mixed lymphocyte assays.

### DETAILED DESCRIPTION

The present invention relates to, in part, the surprising finding that both engineered and non-engineered adipogenic cells can be transplanted into a subject, leading to long-lasting cell engraftment and in vivo secretion of a protein and/or other molecule, such as protein, making them effective for the treatment of diseases or disorders, including diseases or disorders associated with abnormal protein production or complete protein deficiency.

### Adipogenic Cells

Any adipogenic cells are contemplated by the present invention. Non-limiting examples of adipogenic cells include adipocytes, adipogenic stem cells (ASCs), and CD34⁺ cells.

In some embodiments, the adipogenic cells are allogenic. Allogenic cells include cells obtained from a donor that is different from the subject to be treated. In some embodiments, the adipogenic cells are autologous.

In some embodiments, the adipogenic cells are substantially pure.

In embodiments, substantially pure refers to a population of adipogenic cells in which greater than about 80%, or greater than about 85%, greater than about 90%, or greater than about 95%, or greater than about 97%, or greater than about 98%, or greater than about 99% of the cells exhibit the same or similar characteristics (e.g., therapeutic effect, potency, differentiation capacity, mitotic activity, proliferative capacity, morphology, cell-surface markers, and combinations of the foregoing). In embodiments, substantially pure refers to a population of adipogenic cells in which greater than about 80%, or greater than about 85%, greater than about 90%, or greater than about 95%, or greater than about 97%, or greater than about 98%, or greater than about 99% of the cells exhibit the same or similar therapeutic effect. In embodiments, substantially pure refers to a population of adipogenic cells in which greater than about 80%, or greater than about 85%, greater than about 90%, or greater than about 95%, or greater than about 97%, or greater than about 98%, or greater than about 99% of the cells exhibit the same or similar potency. In embodiments, substantially pure refers to a population of adipogenic cells in which greater than about 80%, or greater than about 85%, greater than about 90%, or greater than about 95%, or greater than about 97%, or greater than about 98%, or greater than about 99% of the cells exhibit the same or similar differentiation capacity. In embodiments, substantially pure refers to a population of adipogenic cells in which greater than about 80%, or greater than about 85%, greater than about 90%, or greater than about 95%, or greater than about 97%, or greater than about 98%, or greater than about 99% of the cells exhibit the same or similar mitotic activity. In embodiments, substantially pure refers to a population of adipogenic cells in which greater than about 80%, or greater than about 85%, greater than about 90%, or greater than about 95%, or greater than about 97%, or greater than about 98%, or greater than about 99% of the cells exhibit the same or similar proliferative capacity. In embodiments, substantially pure refers to a population of adipogenic cells in which greater than about 80%, or greater than about 85%, greater than about 90%, or greater than about 95%, or greater than about 97%, or greater than about 98%, or greater than about 99% of the cells exhibit the same or similar morphology.

In embodiments, substantially pure refers to a population of adipogenic cells in which greater than about 80%, or greater than about 85%, greater than about 90%, or greater than about 95%, or greater than about 97%, or greater than about 98%, or greater than about 99% of the cells exhibit the same or similar identity and/or quantity of a cell surface marker.

In embodiments, substantially pure refers to a population of cells which is enriched for adipogenic cells over non-adipogenic cells (e.g. cells of a starting population, cells that are biologically inactive, or cells that hinder the present therapeutic effects). Non-limiting examples of non-adipogenic cells include cells other than adipocytes; depending on the starting cell population, ASCs and/or CD34⁺ cells; and precursor cells thereof that differentiate into non-adipose cells, such as osteoblasts, fibroblasts, lymphocytes, and myeloid cells. In embodiments, substantially pure refers to a population of adipogenic cells which has about 5-fold, or about 10-fold, or about 15-fold, or about 20-fold, or about 30-fold, or about 50-fold, or about 100-fold, or about 300-fold, or about 500-fold, or about 1000-fold more adipogenic cells than non-adipogenic cells.

In embodiments, substantially pure refers to a population of cells which is enriched for adipogenic cells over non-adipogenic cells and which contains one or more helper cells, which increase, enhance, or maintain the present therapeutic effect (e.g. as compared to a population of cells which is enriched for adipogenic cells over non-adipogenic cells and which lacks one or more helper cells).

In some embodiments, the adipogenic cells are cultured and expanded. Methods of culturing are described herein, and would be understood by one of ordinary skill in the art. In some embodiments, adipogenic cells are cultured and expanded to the desired amount of cells. In some embodiments, the composition comprising adipogenic cells is prepared either separately or as co-cultures, in the presence or absence of a matrix or support. In some embodiments, the adipogenic cells are freshly prepared and/or harvested. In some embodiments, the adipogenic cells are thawed from cryopreserved stock. In embodiments, the adipogenic cells are suitable for cryoprotection, e.g. with a cryoprotectant including, e.g. DMSO, albumin (e.g. human serum albumin) and/or saline.

Adipogenic cells may be isolated from any source, as would be understood by one of ordinary skill in the art. In some embodiments, the adipogenic cells are isolated from adipose tissue. In some embodiments, the adipogenic cells are isolated from peripheral blood. In some embodiments, the adipogenic cells are isolated from human peripheral blood. In some embodiments, the adipogenic cells are mammalian adipogenic cells. In some embodiments, the adipogenic cells are human adipogenic cells In some embodiments, the adipogenic cells are suitable for use in a human subject.

In some embodiments, the adipogenic cells are adipocytes. In some embodiments, the adipocytes are brown/beige adipocytes or white adipocytes, or a combination of brown/beige and white adipocytes, e.g, in various ratios.

In some embodiments, the adipogenic cells are a combination of brown/beige adipocytes and white adipocytes. In some embodiments, the ratio of brown/beige adipocytes to white adipocytes is between about 1:99 and about 99:1. In some embodiments, the ratio of brown/beige adipocytes to white adipocytes is between about 1:50 and about 50:1. In some embodiments, the ratio of brown/beige adipocytes to white adipocytes is between about 1:25 and about 25:1. In some embodiments, the ratio of brown/beige adipocytes to white adipocytes is between about 1:10 and about 10:1. In some embodiments, the ratio of brown/beige adipocytes to white adipocytes is between about 1:5 and about 5:1. In some embodiments, the ratio of brown/beige adipocytes to white adipocytes is between about 1:2 and about 2:1. In some embodiments, the ratio of brown/beige adipocytes to white adipocytes is about 1:1.

White adipocytes are found in white adipose tissue, and are adipocytes comprising a single large fat droplet, with a flattened nucleus located on the periphery of the cell. White adipose tissue functions to help maintain body temperature (via insulation) and to store energy in the Form of lipids. White adipose cells can be distinguished from precursor cells by the presence of a C/EBPα and PPARγ2-positive nucleus and high cytoplasmic levels of FABP4 as determined, e.g. by antibody staining. Marker genes of white adipocytes are well known and include, by way of non-limiting example, lipoprotein lipase (LPL; NCBI Gene ID No. 4023), hormone-sensitive lipase (HSL; NCBI Gene ID No. 3991), adiponectin (ADIPOQ NCBI Gene ID No. 9370), FABP4 (NCBI Gene ID No. 2167), CEBPA (NCBI Gene ID No. 1050), and PPARG2 (NCBI Gene ID No. 5468; NCBI Reference Sequence NM-015869), which can be assayed by quantitative RT-PCR.

Brown/beige adipocytes utilize the chemical energy in lipids and glucose to produce heat via non-shivering thermogenesis, and are adipose cells comprising multiple lipid droplets throughout the cell, a rounded nucleus and a large number of mitochondria, which give the cells their distinctive brown color. Marker genes of brown/beige adipocytes are well known and include, by way of non-limiting example, lipoprotein lipase (LPL), UCP1 (NCBI Gene ID No. 7350), ELOVL3 (NCBI Gene ID No. 83401), PGC1A (NCBI Gene ID No. 10891), CYC1 (NCBI Gene ID No. 1537), CEBPA, and PPARG2, which can be assayed by quantitative RT-PCR. Brown/beige adipocytes can be distinguished from white adipocytes by having high relative expression of, by way of non-limiting example, UCP1, ELOVL3, PGC1A, and CYC1 and low relative expression of, by way of non-limiting example, ADIPOO, HSL, and FABP4, while both cell types will display high levels of PPARγ2 and LPL expression.

In some embodiments, the adipocytes express and/or secrete one or more of CIDEC, FABP4, PLIN1, LGALS12, ADIPOQ, TUSC5, SLC19A3, PPARG, LEP, CEBPA, or a combination thereof. In some embodiments, the expression of one or more of CIDEC, FABP4, PLIN1, LGALS12, ADIPOQ, TUSC5, SLC19A3, PPARG, LEP, CEBPA, or a combination thereof, is elevated relative to non-adipocytes, including ASCs and cells from non-adipose tissues.

In some embodiments, the adipocytes and/or adipocytes differentiated from adipocyte precursor cells, such as ASCs or CD34⁺ cells, secrete one or more native products. In some embodiments, the native product is one or more of fatty acids or other fatty acid-derived chemicals. In some embodiments, the fatty acid derived chemicals include fatty acid esters, fatty alkanes and alkenes, fatty alcohols, fatty ketones, and fatty lactones.

In some embodiments, the fatty acid is a saturated or unsaturated fatty acid. In some embodiments, the saturated or unsaturated fatty acid comprises, e.g., at least 8, at least 10, at least 12, at least 14, at least 16, at least 18, at least 20, at least 22, at least 24, at least 26, at least 28, or at least 30 carbon atoms, In some embodiments, the saturated or unsaturated fatty acid comprises, e.g., between 4 and 24 carbon atoms, between 6 and 24 carbon atoms, between 8 and 24 carbon atoms, between 10 and 24 carbon atoms, between 12 and 24 carbon atoms, between 14 and 24 carbon atoms, or between 16 and 24 carbon atoms, between 4 and 22 carbon atoms, between 6 and 22 carbon atoms, between 8 and 22 carbon atoms, between 10 and 22 carbon atoms, between 12 and 22 carbon atoms, between 14 and 22 carbon atoms, or between 16 and 22 carbon atoms, between 4 and 20 carbon atoms, between 6 and 20 carbon atoms, between 8 and 20 carbon atoms, between 10 and 20 carbon atoms, between 12 and 20 carbon atoms, between 14 and 20 carbon atoms, or between 16 and 20 carbon atoms. In some embodiments, the unsaturated fatty acid has, e.g., 1 or more, 2 or more, 3 or more, 4 or more, 5 or more, or 6 or more double bonds. Non-limiting examples of fatty acids include capryllic acid (8:0), pelargonic acid (9:0), capric acid (10:0), undecylic acid (11:0), lauric acid (12:0), tridecylic acid (13:0), myristic acid (14:0), myristoleic acid (14:1), pentadecyclic acid (15:0), palmitic acid (16:0), palmitoleic acid (16:1), sapienic acid (16:1), margaric acid (17:0), stearic acid (18:0), oleic acid (18:1), elaidic acid (18:1), vaccenic acid (18:1), linoleic acid (18:2), linoelaidic acid (18:2), a-linolenic acid (18:3), y-linolenic acid (18:3), stearidonic acid (18:4), nonadecylic acid (19:0), arachidic acid (20:0), eicosenoic acid (20:1), dihomo-y-linolenic acid (20:3), mead acid (20:3), arachidonic acid (20:4), eicosapentaenoic acid (20:5), heneicosylic acid (21:0), behenic acid (22:0), erucic acid (22:1), docosahexaenoic acid (22:6), tricosylic acid (23:0), lignoceric acid (24:0), nervonic acid (24:1), pentacosylic acid (25:0), cerotic acid (26:0), heptacosylic acid (27:0), montanic acid (28:0), nonacosylic acid (29:0), melissic acid (30:0), henatriacontylic acid (31:0), lacceroic acid (32:0), psyllic acid (33:0), geddic acid (34:0), ceroplastic acid (35:0), and hexatriacontylic acid (36:0).

In some embodiments, adipocytes are characterized as having one or more, 2 or more, 3 or more, 4 or more, 5 or more, 10 or more, 15 or more, 20 or more, 25 or more, 30 or more, or 35 or more of the following:
a. being post-mitotic;
b. having a lipid content of greater than about 35% (% fresh weight of adipose tissue; e.g. greater than about 40%, about 45%, about 50%, about 55%, about 60%, about 65%, about 70%, about 75%, or about 80%);
c. having a fat content in adipose tissue of about 60% to about 95% (e.g. 60-94%, about 60% to about 90%, about 60% to about 85%, about 60% to about 80%, about 60% to about 75%, about 60% to about 70%, about 60% to about 65%, about 65% to about 90%, about 70% to about 90%, about 75% to about 90%, about 80% to about 90%, or about 85% to about 90%);
d. having an average fat content of about 80% (e.g. about 75 to about 85%);
e. having a water content in adipose tissue of about 5% to about 40% (e.g. about 6-36%, about 5% to about 35%, about 5% to about 30%, about 5% to about 25%, about 5% to about 20%, about 5% to about 15%, about 5% to about 10%, about 10% to about 40%, about 15% to about 40%, about 20% to about 40%, about 25% to about 40%, about 30% to about 40%, or about 35% to about 40%);
f. having an average water content of about 15% (e.g. about 12.5% to about 17.5%);
g. having a specific gravity of about 1 g/mL (e.g. 0.916 g/mL, about 0.5 g/mL, about 0.6 g/mL, about 0.7 g/mL, about 0.8 g/mL, about 0.9 g/mL, about 1.1 g/mL, or about 1.2 g/mL);
h. having a lipid content comprising one or more of stearic acid, oleic acid, linoleic acid, palmitic acid, palmitoleic acid, and myristic acid, a derivative thereof;
i. having a lipid content comprising one or more of free fatty acids, cholesterol, monoglycerides, and diglycerides;
j. having a lipid droplet of a size greater than about 90% of the cell volume (e.g. greater than 95% or greater than about 98%, or about 93%, or about 95%, or about 97%, or about 99%);
k. having a lipid droplet comprising at least about 30% to about 99% of the volume of the cell; (e.g., at least about 40% to about 90%, about 50% to about 90%, about 60% to about 90%, about 70% to about 90% about 80% to about 90%, about 50%, about 60%, about 70%, about 80%, or about 90%);
l. having a surface size of about 20-300 μm in diameter (e.g. about 20-300 μm, about 20-200 μm, about 20-100 μm, about 20-500 μm, about 20-30 μm, about 50-300 μm, about 50-200 μm, about 50-100 μm, about 100-300 μm, about 100-200 μm, about 150-300 μm, about 150-200 μm, or about 200-300 μm);
m. having a nucleus volume of about 200-400 μm³ (e.g. about 200 to about 350 μm³, about 200 to about 300 μm³, about 200 to about 250 μm³, about 250 to about 400 μm³, about 250 to about 350 μm³, about 250 to about 300 μm³, about 300 to about 350 μm³ or about 300 to about 400 μm³);
n. having a total volume of about 4,000-18,000 μm³ (e.g. about 4000 to about 15000 μm³, about 5000 to about 15000 μm³, about 10000 to about 15000 μm³, about 12500 to about 15000 μm³, about 4000 to about 10000 μm³, about 5000 to about 15000 μm³, about 7500 to about 15000 μm³, about 10000 to about 15000 μm³, about 12500 to about 15000 μm³);
o. having a nucleus to cell ratio of about 1:20-1:90 (e.g. about 1:20 to about 1:80, about 1:20 to about 1:70, about 1:20 to about 1:60, about 1:20 to about 1:50, about 1:20 to about 1:40, about 1:20 to about 1:30; about 1:30 to about 1:80, about 1:40 to about 1:80, about 1:50 to about 1:80, about 1:60 to about 1:80, or about 1:70 to about 1:80);
p. having a flattened nucleus;
q. having a small cytoplasm of less than about 10% to about 60% of total cell volume, wherein the cytoplasm excludes lipid droplets volume (e.g. less than about 20%, less than about 30%, less than about 40%, or less than about 50%);
r. being capable of absorbing and releasing liquids;
s. being buoyant in in water or an aqueous solution (e.g., media, or HBSS);
t. having a non-centrally located nucleus;
u. having one or more fat droplets;
v. having a non-spherical cytoplasm;
w. being capable of secreting one or more of adiponectin, leptin, and TNF-alpha;
x. being capable of lipogenesis;
y. being capable of storing triglycerides (TG);
z. being capable of secreting non-esterified fatty acids (NEFA) (e.g., long chain fatty acids such as oleic acid palmitoleic acid, linoleic acid, arachidonic acid, lauric acid, and stearic acid);
aa. being responsive to hormones;
bb. being responsive to neural input;
cc. having a cell turn-over rate of about 9 years (e.g. about 8 to about 10 years);
dd. having an average diameter of about 45 μm (e.g. about 47.2 μm, about 40 μm, about; 42.5 μm, about 47.5 μm, or about 50 μm)
ee. a cell population having a diameter distribution wherein about 25% of cells have a diameter of less than about 50 μm; about 40% of cells have a diameter of about 50-69 μm; about 25% of cells have a diameter of about 70-89 μm, and about 10% of cells have a diameter of greater than or equal to about 90 μm;
ff. responsive to atrial natriuretic peptide (ANP);
gg. capable of lipolysis;
hh. expressing receptors that can bind and respond to steroid hormones;
ii. lysed due to phosphatidylcholine;
jj. cell density of about 1 g/ml (e.g. about 0.8 g/ml, about 0.9 g/ml, about 1.1 g/ml, about 1.2 g/ml);
kk. greater than about 80% viability (e.g. about 85%, about 90%, about 95%, about 97%, about 98%, or about 99%);
ll. greater than about 80% purity (e.g. about 85%, about 90%, about 95%, about 97%, about 98%, or about 99%),
mm. adequate potency (e.g. amount of Oil Red O eluted greater than about 200 μg/ml); and
nn. negative for microbial contamination.
   See, for example, Thomas, Quarterly Journal of Experimental Physiology and Cognate Medical Sciences, 47, 2, 179-188 (1962), ICRP Publication 23, Report of the Task Group on Reference Man (1975), John Blarmire, BIOdotEDU: Components of cells; The macromolecules; Adipose tissue (2005), Stenkula and Erlanson-Albertsson, Am J Physiol Regul Integr Comp Physiol 315, R284-R295 (2018); Ambati et al., MBC Obes. 3, 35 (2016); Charo et al., Nucleus, 7, 3, 249-269 (2016); Shoham et al., Biophys J., 106, 6, 1421-1431 (2014); Verboven et al., Scientific Reports 8, 4677 (2018); all of which are incorporated by reference herein in their entireties.

In some embodiments, the adipocytes are capable of lipogenesis. Any method for identifying and/or measuring lipogenesis is contemplated by the present invention. For example, lipogenesis can be determined by measuring for the expression of genes involved in de novo lipogenesis (DNL) and in fatty acid elongation and desaturation. In another example, ¹³C-labeled substrates can be utilized to study the pathway of DNL. In a non-limiting example, human adipocytes differentiated with no exogenous fat accumulated triacylglycerol (TG) in lipid droplets and differentiated normally. TG composition showed the products of DNL (saturated fatty acids from 12:0 to 18:0) together with unsaturated fatty acids (particularly 16:1n-7 and 18:1n-9) produced by elongation/desaturation. See, for example, Collins et al. J. Lipid Res. 52, 9, 1683-1692 (2011), which is incorporated by reference herein in its entirety. For other examples of methods for identifying and/or measure lipogenesis, see Müller, Drug Discovery and Evaluation: Pharmacological Assays, Springer International Publishing Switzerland (2016), which is incorporated by reference herein in its entirety.

In some embodiments, the adipocytes are responsive to hormones. Non-limiting examples of hormones include glucocorticoids, estrogens, steroid hormones such as androgens, adrenaline, noradrenaline, amino acid derivative hormones such as triiodothyronine, adrenocorticotropic hormone-releasing factor, thyroid-stimulating hormone-releasing factor, somatostatin, luteinizing hormone, growth Hormones, peptide hormones such as leucine enkephalin, oxytocin, vasopressin, glucagon, insulin, secretin, and calcitonin. Any method for identifying and/or measuring responsiveness to hormones is contemplated by the present invention. For non-limiting examples of methods, see Müller, Drug Discovery and Evaluation: Pharmacological Assays, Springer International Publishing Switzerland (2016), which is incorporated by reference herein in its entirety.

In some embodiments, the adipocytes are responsive to neural input. Any method for identifying and/or measuring responsiveness to neural input is contemplated by the present invention. For non-limiting examples of methods, see Correll, Science 140, 26, 387-388 (1963), which is incorporated by reference herein in its entirety.

In some embodiments, the adipocytes are responsive to atrial natriuretic peptide (ANP). Any method for identifying and/or measuring responsiveness to ANP is contemplated by the present invention. For non-limiting examples of methods, see Verboven et al., Scientific Reports 8, 4677 (2018), which is incorporated by reference herein in its entirety.

In some embodiments, the adipocytes are capable of lipolysis. Any method for identifying and/or measuring lipolysis is contemplated by the present invention. Non-limiting examples of methods for cellular lipolysis, cell-free lipolysis, and analysis of lipolysis products can be found in Müller, Drug Discovery and Evaluation: Pharmacological Assays, Springer International Publishing Switzerland (2016), which is incorporated by reference herein in its entirety.

In some embodiments, the adipocytes express receptors that can bind and respond to steroid hormones. Any method for identifying and/or measuring the expression of receptors that can bind and respond to steroid hormones is contemplated by the present invention. For non-limiting examples of methods, see Rebuffé-Scrive et al., J. Clin. Endocrinol. Metab. 71, 5, 1215-1219 (1990), which is incorporated by reference herein in its entirety.

In some embodiments, the adipocytes are lysed due to phosphatidylcholine. Any method for identifying and/or measuring lysis due to phosphatidylcholine is contemplated by the present invention. For non-limiting examples of methods, see Kim et al., PLoS One 12, 5, e0176722 (2017), which is incorporated by reference herein in its entirety.

In some embodiments, the adipogenic cells are ASCs. In some embodiments, the ASCs are mammalian ASC. Non-limiting examples of mammalian ASCs include primate ASCs (such as human ASCs). In some embodiments, the ASCs have one or more, or one, two, three of:
(a) a viability of about 90% or greater;
(b) a glucose uptake of about 5 mmol/L to about 10 mmol/L (e.g. about 6.13±0.58 mmol/L to about 7.73±0.37 mmol/L, about 5 mmol/L to about 7.5 mmol/L, about 2.5 mmol/L to about 10 mmol/L, about 2.5 mmol/L to about 7.5 mmol/L, or about 2.5 mmol/L to about 5 mmol/L; and
(c) a lactate production of about 10 mmol/L to about 15 mmol/L (e.g. about 10.53±1.09 mmol/L to about 12.91±1.12 mmol/L, about 10 mmol/L to about 14 mmol/L, about 10 mmol/L to about 13 mmol/L, about 10 mmol/L to about 12 mmol/L, about 10 mmol/L to about 11 mmol/L, about 10 mmol/L to about 14 mmol/L, about 10 mmol/L to about 13 mmol/L, about 10 mmol/L to about 12 mmol/L, about 10 mmol/L to about 15 mmol/L).
   See, for example, Kolodziej et al., Adipocyte 8, 1, 254-264 (2019), which is incorporated by reference herein in its entirety.

In some embodiments, the ASCs are highly adipogenic. For example, highly adipogenic ACSs can be the strongest responder to adipogenic differentiation and/or yield significantly more adipocytes both in vitro and in vivo relative to control ASCs. In some embodiments, highly adipogenic ASCs are isolated through selection for cell surface proteins that are differentially expressed between the highly adipogenic ASCs and control ASCs. In some embodiments, the highly adipogenic ACS show high or elevated expression levels of upregulated adipocyte-specific genes relative to ASCs isolated from adipose tissue without selection (e.g., in embodiments, about 2-fold, or about 5-fold, or about 10-fold, or about 30-fold, or about 100-fold). Non-limiting examples of genes that can be upregulated in highly adipogenic cells include MAT2B, CCDC115, CCDC69, SLC2A3, SPPL3, CD107b (LAMP2), GINM1, CDw210 (IL10RB), CD164, and CD253 (TNFSF10) compared to wild type adipogenic cells and/or unenriched adipogenic cells and/or are obtainable from ASCs that expresses elevated levels of the genes compared to wild type ASCs and/or unenriched ASCs. In some embodiments, the highly adipogenic ACS show reduced expression levels of downregulated adipocyte-specific genes relative to ASCs isolated from adipose tissue without selection. Non-limiting examples of genes that can be downregulated in highly adipogenic cells include MAP11, UBASH3B, NCS1, TRAF7, GNB2, ANO10, FKBP2, EMP3, CD266 (TNFRSF12A), CD151, CD49c (ITGA3), and CD91 (LRP1) compared to wild type adipogenic cells and/or unenriched adipogenic cells and/or are obtainable from ASCs that expresses elevated levels of the genes compared to wild type ASCs and/or unenriched ASCs. In some embodiments, highly adipogenic ACSs can be isolated in vitro or in vivo.

In some embodiments, the ASCs exhibit upregulation of one or more of MAT2B, CCDC115, CCDC69, SLC2A3, SPPL3, CD107b (LAMP2), GINM1, CDw210 (IL10RB), CD164, and CD253 (TNFSF10) compared to wild type ASCs and/or unenriched ASCs. In some embodiments, the ASCs exhibit upregulation of one or more of MAT2B, CCDC69, CDw210 (IL10RB), CD107b (LAMP2), CD164, and CD253 (TNFSF10) compared to wild type ASCs and/or unenriched ASCs. In some embodiments, the ASCs exhibit upregulation of one or more of MAT2B, CCDC69, CDw210 (IL10RB), and CD164 compared to wild type ASCs and/or unenriched ASCs. In some embodiments, the ASCs exhibit upregulation of one or more of one or more of CDw210, CD107b, CD164, and CD253 compared to wild type ASCs and/or unenriched ASCs.

In some embodiments, the ASCs exhibit downregulation of one or more of MAP11, UBASH3B, NCS1, TRAF7, GNB2, ANO10, FKBP2, EMP3, CD266 (TNFRSF12A), CD151, CD49c (ITGA3), and CD91 (LRP1) compared to wild type ASCs and/or unenriched ASCs. In some embodiments, the ASCs exhibit downregulation of one or more of UBASH3B, CD266 (TNFRSF12A), CD151, and CD49c (ITGA3). compared to wild type ASCs and/or unenriched ASCs. In some embodiments, the ASCs exhibit downregulation of one or more of UBASH3B and CD266 (TNFRSF12A compared to wild type ASCs). In some embodiments, the ASCs exhibit downregulation of one or more CD266, CD151, CD49c, and CD9 compared to wild type ASCs and/or unenriched ASCs. In some embodiments, the ASCs exhibit downregulation of CD266, CD151, CD49c, and CD9 compared to wild type ASCs and/or unenriched ASCs.

In some embodiments, the ASCs express elevated levels of one or more of CDw210, CD107b, CD164, and CD253 compared to, e.g., wild type ASCs and/or unenriched ASCs. In some embodiments, the ASCs express and/or secrete reduced levels of one or more of CD266, CD151, CD49c, and CD9 compared to, e.g., wild type ASCs and/or unenriched ASCs. In some embodiments, the ASCs express elevated levels of one or more of CDw210, CD107b, CD164, and CD253, and express reduced levels of one or more of CD266, CD151, CD49c, and CD9 compared to wild type ASCs and/or unenriched ASCs. In some embodiments, the ASCs are negative for CD266, CD167, CD325, and CD115 and positive for one or more of CD361, CD120b, CD164, and CD213A1 compared to wild type ASCs and/or unenriched ASCs.

In some embodiments, the ASCs differentiate into adipocytes that secrete high levels of adiponectin. For example, the adipocytes express 2.5-10 times more adiponectin than the average adipocyte (e.g. wild type adipocytes and/or unenriched adipocytes). In some embodiments, these ASCs are isolated through selection for plasma membrane proteins that are differentially expressed between them and control ASCs. In some embodiments, the ASCs differentiate into adipocytes that secrete high levels of adiponectin are highly adipogenic. Non-limiting examples of genes that can be upregulated (e.g., in embodiments, about 2-fold, or about 5-fold, or about 10-fold, or about 30-fold, or about 100-fold) in ASCs that differentiate into adipocytes that secrete high levels of adiponectin include GINM1, CCDC69, CCDC115, CD361 (EV12B), CD120b (TNFRSF1B), CD164, CD213A1 (IL13RA1), and CD10 compared to wild type ASCs and/or unenriched ASCs. Non-limiting examples of genes that can be downregulated (e.g., in embodiments, about 2-fold, or about 5-fold, or about 10-fold, or about 30-fold, or about 100-fold) in ASCs that differentiate into adipocytes that secrete high levels of adiponectin include FKBP2, THBS1, CTNNB1, MPZL1, CD266 (TNFRSF12A), CD167 (DDR1), CD325 (CDH2), and CD115 (PVR) compared to wild type ASCs and/or unenriched ASCs. In some embodiments, the ACSs can be isolated in vitro or in vivo.

In some embodiments, the ASCs exhibit upregulation of one or more of GINM1, CCDC69, CCDC115, CD361 (EV12B), CD120b (TNFRSF1B), CD164, CD213A1 (IL13RA1), and CD10 compared to wild type ASCs and/or unenriched ASCs. In some embodiments, the ASCs exhibit upregulation of one or more of CDC69, CD361 (EV12B), CD120b (TNFRSF1B), CD164, and CD213A1 (IL13RA1) compared to wild type ASCs and/or unenriched ASCs. In some embodiments, the ASCs exhibit upregulation of CDC69, CD361 (EV12B), CD164, and CD213A1 (IL13RA1) compared to wild type ASCs and/or unenriched ASCs. In some embodiments, the ASCs exhibit upregulation of one or more of CD361, CD120b, CD164, and CD213A1 compared to wild type ASCs and/or unenriched ASCs.

In some embodiments, the ASCs exhibit downregulation of one or more of FKBP2, THBS1, CTNNB1, MPZL1, CD266 (TNFRSF12A), CD167 (DDR1), CD325 (CDH2), and CD115 (PVR) compared to wild type ASCs and/or unenriched ASCs. In some embodiments, the ASCs exhibit downregulation of one or more of CD266 (TNFRSF12A), CD167 (DDR1), CD325 (CDH2), and CD115 (PVR) compared to wild type ASCs and/or unenriched ASCs. In some embodiments, the ASCs exhibit downregulation of one or more of CD266 (TNFRSF12A) and CD325 (CDH2) compared to wild type ASCs and/or unenriched ASCs. In some embodiments, the ASCs exhibit downregulation of CD266, CD167, CD325, and CD115 compared to wild type ASCs and/or unenriched ASCs.

In some embodiments, the ASCs express elevated levels of one or more of CD361, CD120b, CD164, and CD213A1 compared to, e.g., wild type ASCs and/or unenriched ASCs. In some embodiments, the ASCs express reduced levels of one or more of CD266, CD167, CD325, and CD115 compared to wild type ASCs and/or unenriched ASCs. In some embodiments, the ASCs express elevated levels of one or more of CD361, CD120b, CD164, and CD213A1, and express reduced levels of one or more of CD266, CD167, CD325, and CD115 compared to wild type ASCs and/or unenriched ASCs. In some embodiments, the ASCs are negative for CD151, CD10, CD26, and CD142 and positive for one or more of CDw210b, CD340 and CDw293 compared to wild type ASCs and/or unenriched ASCs.

In some embodiments, the ASCs exhibit upregulation of CD10 compared to, e.g., wild type ASCs and/or unenriched ASCs. In some embodiments, ASCs exhibiting upregulation of CD10 express and/or secrete elevated levels of adiponectin compared to, e.g., wild type ASCs and/or unenriched ASCs. In some embodiments, ASCs exhibiting upregulation of CD10 express and/or secrete levels of adiponectin about 1.5-fold, or about 2-fold, or about 5-fold, or about 10-fold, or about 30-fold, or about 100-fold greater than wild type ASCs and/or unenriched ASCs. In some embodiments, about 1% to about 99%, about 50% to about 99%, about 75% to about 99%, or about 80% to about 99% of the ASCs express CD10 compared to wild type ASCs and/or unenriched ASCs. In some embodiments, at least about 60%, 65%, 70%, 75%, 80%, 85%, 90%, 95%, 96%, 97%, 98%, 99%, or greater than 99% of the ASCs express CD10 compared to wild type ASCs and/or unenriched ASCs.

In some embodiments, the ASCs are selectively enriched for one or more of CD10, CDw210, CD107b, CD164, CD253, CD361, CD120b, CD213A1, HLAII, CDI Ib, CDI Ic, CD14, CD45, CD31, CD34, CD80 and CD86. Non-limiting methods for selectively enriching ASCs include, but are not limited to, antibody-based methods, such as affinity capture and FACS. In some embodiments, the ASCs and/or a population of ASCs are selectively enriched for CD10 (e.g. CD10-enriched ASCs). In some embodiments, CD10-enriched ASCs express elevated levels of CD10 compared to wild type ASCs and/or unenriched ASCs. In some embodiments, about 1% to about 99%, about 50% to about 99%, about 75% to about 99%, or about 80% to about 99% of the CD10-enriched ASCs express CD10. In some embodiments, at least about 60%, 65%, 70%, 75%, 80%, 85%, 90%, 95%, 96%, 97%, 98%, 99%, or greater than 99% of the CD10-enriched ASCs express CD10 compared to wild type ASCs and/or unenriched ASCs.

In some embodiments, the adipogenic cells of the disclosure are obtainable from CD10-enriched ASCs. In a non-limiting example, CD10-enriched ASCs differentiate into adipogenic cells (e.g. brown/beige adipocytes or white adipocytes) that express CD10. the adipogenic cells are white adipocytes obtainable from CD10-enriched ASCs. In some embodiments, the ASCs express elevated levels of CD10 compared to wild type ASCs and/or unenriched ASCs. In some embodiments, about 1% to about 99%, about 50% to about 99%, about 75% to about 99%, or about 80% to about 99% of the CD10-enriched ASCs express CD10. In some embodiments, at least about 60%, 65%, 70%, 75%, 80%, 85%, 90%, 95%, 96%, 97%, 98%, 99%, or greater than 99% of the CD10-enriched ASCs express CD10 compared to wild type ASCs and/or unenriched ASCs.

In some embodiments, the ASCs produce adipocytes expressing high levels of intracellular PEX5. For example, the adipocytes give rise to adipocytes expressing PEX5 at levels higher than 75% of the population. In some embodiments, ASCs that produce adipocytes expressing high levels of intracellular PEX5 are highly adipogenic. In some embodiments, these ASCs are isolated through selection for plasma membrane proteins that are differentially expressed between them and control ASCs. Non-limiting examples of genes that can be upregulated (e.g., in embodiments, about 2-fold, or about 5-fold, or about 10-fold, or about 30-fold, or about 100-fold) in ASCs that produce adipocytes expressing high levels of intracellular PEX5 include LRRFIP2, AVEN, SHKBP1, SMPD2, CDw210b (IL10RB), CD340 (ERBB2), and CDw293 (BMPR1B) compared to wild type ASCs and/or unenriched ASCs. Non-limiting examples of genes that can be downregulated in ASCs that produce adipocytes expressing high levels of intracellular PEX5 include TGA7, PLEKHG4, SYNC, CD151, CD10 (MME), CD26 (DPP4), and CD142 (F3) compared to wild type ASCs and/or unenriched ASCs. In some embodiments, the ACSs can be isolated in vitro or in vivo.

In some embodiments, the ASCs exhibit upregulation of one or more of LRRFIP2, AVEN, SHKBP1, SMPD2, CDw210b (IL10RB), CD340 (ERBB2), and CDw293 (BMPR1B) compared to wild type ASCs and/or unenriched ASCs. In some embodiments, the ASCs exhibit upregulation of one or more of CDw210b (IL10RB), CD340 (ERBB2), and CDw293 (BMPR1B) compared to wild type ASCs and/or unenriched ASCs.

In some embodiments, the ASCs exhibit downregulation of one or more of TGA7, PLEKHG4, SYNC, CD151, CD10 (MME), CD26 (DPP4), and CD142 (F3). compared to wild type ASCs and/or unenriched ASCs. In some embodiments, the ASCs exhibit downregulation of one or more of CD151, CD10 (MME), CD26 (DPP4), and CD142 (F3) compared to wild type ASCs and/or unenriched ASCs. In some embodiments, the ASCs exhibit downregulation of CD115 (PVR). In some embodiments, the ASCs exhibit downregulation of CD151, CD10 (MME), CD26 (DPP4), and CD142 (F3) compared to wild type ASCs and/or unenriched ASCs.

In some embodiments, the ASCs express elevated levels of one or more of CDw210b, CD340 and CDw293 compared to, e.g., wild type ASCs and/or unenriched ASCs. In some embodiments, the ASCs express reduced levels of one or more of CD151, CD10, CD26, and CD142 compared to, e.g., wild type ASCs and/or unenriched ASCs. In some embodiments, the ASCs express elevated levels of one or more of CDw210b, CD340 and CDw293, and express reduced levels of one or more of CD151, CD10, CD26, and CD142 compared to wild type ASCs and/or unenriched ASCs. In some embodiments, the ASCs are negative for CD151, CD10, CD26, and CD142 and positive for one or more of CDw210b, CD340 and CDw293 compared to wild type ASCs and/or unenriched ASCs.

In some embodiments, less than about 10%, about 9%, about 8%, about 7%, about 6%, about 5%, about 4% about 3% about 2% or about 1% of ASCs express one or more of the surface markers HLAII, CDI Ib, CDI Ic, CD14, CD45, CD31, CD34, CD80 and CD86. In some embodiments, less than about 5% of ASCs express one or more of the surface markers HLAII, CDI Ib, CDI Ic, CD14, CD45, CD31, CD34, CD80 and CD86.

In some embodiments, at least about 85%, about 86%, about 87%, about 88%, about 89%, about 90%, about 91%, about 92%, about 93%, about 94%, about 95%, about 96%, about 97%, about 98%, or about 99% of the ASCs express one or more of the surface markers HLA I, CD29, CD44, CD59, CD73, CD90, and CD105. In some embodiments, at least about 90% of the ASCs express one or more of the surface markers HLA I, CD29, CD44, CD59, CD73, CD90, and CD105. In some embodiments, at least about 95% of the ASCs express one or more of the surface markers HLA I, CD29, CD44, CD59, CD73, CD90, and CD105.

In some embodiments, the adipogenic cells are CD34⁺ cells. In some embodiments, the CD34⁺ cells are obtained from peripheral blood stem cell (PBSC) donations. In some embodiments, the CD34⁺ cells are obtained from bone marrow transplants (BMT). In some embodiments, the donor has a body mass index (BMI) of less than 20, less than 25, less than 30, less than 35, or less than 40.

In some embodiments, the adipogenic cells are adipocyte precursor cells that differentiate into adipocytes. In some embodiments, the adipogenic cells differentiate into adipocytes in vitro. In some embodiments, the adipogenic cells differentiate into adipocytes in vivo. In some embodiments, the adipocytes exhibit higher expression levels of the adipogenic genes compared to the adipocyte precursor cells.

In some embodiments, the adipogenic cells comprise adipocyte precursor cells. As would be understood by one of ordinary skill in the art, adipocyte precursor cells include cells that differentiate into adipocytes. Non-limiting examples of adipocyte precursor cells include adipogenic stem cells (ASCs) and CD34⁺ cells. In some embodiments, the adipocyte precursor cells comprise ASCs. In some embodiments, the adipocyte precursor cells comprise CD34⁺ cells. In some embodiments, the adipocyte precursor cells comprise ASCs and CD34⁺ cells.

In some embodiments, the adipogenic cells, upon administration to a subject, provide a therapeutically effective amount of adipocytes. In some embodiments, the adipogenic cells comprise adipocyte precursor cells which differentiate into adipocytes in vitro, and a therapeutically effective amount of the adipocytes is administered to a subject. In some embodiments, the adipogenic cells comprise adipocyte precursor cells, which differentiate into adipocytes in vivo to provide a therapeutically effective amount of adipocytes.

In some embodiments, the percentage of adipogenic cells that differentiate into adipocytes is about 1% to about 99% or more, about 20% to about 90%, or about 50% to about 80%. In some embodiments, about 50% to about 80% of adipogenic cells differentiate into adipocytes. In some embodiments, more than about 40%, about 45%, about 50%, about 55%, about 60%, about 65%, about 70%, about 75%, about 80%, about 85%, about 90%, about 95%, or more than 99% of adipogenic cells differentiate into adipocytes. In some embodiments, more than about 80% of adipogenic cells differentiate into adipocytes.

In some embodiments, the adipogenic cells are non-immunogenic. In some embodiments, the adipogenic cells do not trigger and/or do not substantially trigger an innate immune response in a subject. Non-limiting methods for identifying an innate immune response include measuring the level of factors indicative of an innate immune response including, but not limited to, TNFα, IFNγ, IL1R, IL6, IL10, and IL2, using any method as would be understood by one of ordinary skill in the art. In some embodiments, adipogenic cells of the disclosure result in no upregulation and/or substantially no upregulation of one or more factors selected from TNFα, IFNγ, IL1R, IL6, IL10, and IL2 in a subject. In some embodiments, adipogenic cells of the disclosure result in a reduced level of one or more factors selected from TNFα, IFNγ, IL1R, IL6, IL10, and IL2 in a subject compared to a subject exhibiting an innate immune response.

In some embodiments, the adipogenic cells are transplanted into a subject in need thereof. In some embodiments, the transplanted adipogenic cells comprise adipocyte precursor cells, such as ASCs and CD34⁺ cells. In some embodiments, adipogenic cells differentiate into adipocytes upon transplantation. In some embodiments, the transplanted adipogenic cells comprise adipocytes. In some embodiments, the adipocytes are engrafted after transplantation. Methods for determining adipocyte engraftment are described herein and include, without limitation, measuring above-baseline levels of protein expressed by the adipocytes. In some embodiments, the biodistribution of the adipogenic cells can be controlled and measured. In some embodiments, the biodistribution of adipocytes derived from transplanted ASCs is localized at the site of transplantation. In some embodiments, the biodistribution of adipocytes derived from transplanted CD34⁺ cells is widespread throughout the body.

In one aspect, adipocyte precursor cells are transplanted into a subject at a volumetric dose. In some embodiments, adipocyte precursor cells at a concentration of about 250,000 cells/kg to about 4 million cells/kg are suspended in water or other suitable buffer (e.g. PBS, HBSS, etc.), and the adipocyte precursor cells are transplanted into a subject at a dose of about 0.01 μL to about 100 mL, about 0.1 μL to about 10 mL, about 1 μL to about 3 mL, or about 100 μL to about 2 mL. In some embodiments, the adipocyte precursor cells are transplanted into a subject at a dose of about 0.00001 cc to about 100 cc, about 0.0001 cc to about 10 cc, about 0.001 cc to about 3 cc, or about 0.1 cc to about 2 cc. In some embodiments, the adipocyte precursor cells are ASCs. In some embodiments, the adipocyte precursor cells are CD34⁺ cells.

In some embodiments, adipogenic cells and/or adipocyte precursor cells are transplanted and/or implanted into a subject using a needle. Any needle size and/or needle gauge that is useful for transplanting and/or implanting the cells of the disclosure is contemplated by the present disclosure. In some embodiments, the needle has a gauge of 25 G or larger, 26 G or larger, 27 G or larger, 28 G or larger, 29 G or larger, or 30 G or larger. In some embodiments, the needle gauge is 25 G, 26 G, 27 G, 28 G, 29 G, or 30 G.

In one aspect, the adipogenic cells of the present invention exhibit long-lasting cell engraftment and secretion of adiponectin in vivo. Methods of determining the engraftment of adipogenic cells are described herein and include, without limitation, monitoring the serum level of adiponectin since adiponectin is specific to adipocytes, assessing the presence of adipocytes in harvested tissues, and analyzing bone marrow using flow cytometry for the presence of differentiated adipocytes. In some embodiments, the percentage of engraftment ranges from about 10% to about 99%. In some embodiments, the percentage of engraftment ranges from about 20% to about 80%. In some embodiments, the percentage of engraftment is at least 10%, 20%, 30%, 40%, 50%, 60%, 70%, 80%, 90%, 95%, or 99% or more.

In some embodiments, the adipogenic cells persist up to 1 day, up to 2 days, up to 3 days, up to 4 days, up to 5 days, up to 6 days, up to 7 days, up to 2 weeks, up to 3 weeks, up to 1 month, up to 2 months, up to 3 months, up to 4 months, up to 5 months, up to 6 months, up to 7 months, up to 8 months, up to 9 months, up to 10 months, up to 11 months, up to 1 year, or up to 2 years post engraftment, or more, e.g., at least: 3, 4, 5, 6, 7, 8, 9, or 10 years. In some embodiments, the adipogenic cells secrete a molecule (e.g. protein) of interest up to 1 day, up to 2 days, up to 3 days, up to 4 days, up to 5 days, up to 6 days, up to 7 days, up to 2 weeks, up to 3 weeks, up to 1 month, up to 2 months, up to 3 months, up to 4 months, up to 5 months, up to 6 months, up to 7 months, up to 8 months, up to 9 months, up to 10 months, up to 11 months, up to 1 year, or up to 2 years post engraftment, or more, e.g., at least: 3, 4, 5, 6, 7, 8, 9, or 10 years.

In some embodiments, the adipogenic cells of the present invention have enhanced viability. Viability of the adipogenic cells of the present invention can be determined using any methods known in the art, including, without limitation, the examination of membrane integrity with colorimetric or fluorescent dyes. In some embodiments, the adipogenic cells are at least about 60%, about 65%, about 70%, about 75%, about 80%, about 85%, about 90%, about 91%, about 92%, about 93%, about 94%, about 95%, about 96%, about 97%, about 98%, or about 99% or more viable.

### Engineered and Unengineered Adipogenic Cells

In one aspect, the present invention includes engineered adipogenic cells. Non-limiting methods for genetically engineering adipogenic cells are described herein. For example, lentivirus vectors can be used to genetically modify adipogenic cells. In some embodiments, the engineered adipogenic cells include engineered adipocytes and/or engineered adipocyte precursor cells (such as engineered ASCs or engineered CD34⁺ cells).

In some embodiments, adipocyte precursor cells, such as ASCs and/or CD34⁺ cells, are first engineered to express and/or secrete a protein of interest upon differentiation into adipocytes. In some embodiments, the adipogenic cells comprise engineered ASCs. In some embodiments, the adipogenic cells comprise engineered CD34⁺ cells. In some embodiments, the engineered adipogenic cells differentiate into adipocytes in vitro. In some embodiments, the engineered adipogenic cells differentiate into adipocytes in vivo. In some embodiments, the adipogenic cells are engineered to express and/or secrete a reporter protein upon differentiation into adipocytes. A non-limiting example of a reporter protein is Gaussia luciferase (GLuc). In some embodiments, the adipogenic cells is engineered to express and/or secrete a mammalian serum protein upon differentiation into adipocytes. A non-limiting example of a serum protein is erythropoietin (EPO). In some embodiments, the adipogenic cells is engineered to express and/or secrete an intracellular mammalian protein, such as an intracellular enzyme, upon differentiation into adipocytes. A non-limiting example of an intracellular mammalian protein is phenylalanine hydroxylase (PAH). Other non-limiting examples of proteins that can be expressed and/or secreted by engineered adipogenic cells include Cystinosin, GLP-1, Factor VIII, Factor IX, COL2A1, Parathyroid hormone (1-84), alkaline phosphatase, alpha-1 antitrypsin, Trastuzumab, Apolipoprotein A1, Isobutyryl-CoA dehydrogenase, SLC25A20, ATP-binding cassette sub-family G member 5, ABCG5, Phenylalanine hydroxylase, Xanthine dehydrogenase, Ornithine-transcarbamoylase, 3-Hydroxy-3-methylglutaryl-CoA synthase, Glycine cleavage system P protein, Lysine:α-ketoglutarate reductase, Cystathionine β-synthase, Phytanoyl-CoA hydroxylase, and human growth hormone (somatotropin), adipsin, adiponectin. In some embodiments, the protein expressed and/or secreted by engineered adipogenic cells is erythropoietin (EPO). In some embodiments, the protein expressed and/or secreted by engineered adipogenic cells is selected from erythropoietin (EPO), adipsin, and adiponectin.

In one aspect, the present invention includes unengineered adipogenic cells. In some embodiments, the unengineered adipogenic cells include unengineered adipocytes and/or unengineered adipocyte precursor cells (such as unengineered ASCs or unengineered CD34⁺ cells). Non-limiting methods for identifying and isolating unengineered adipogenic cells are described herein. In some embodiments, the unengineered adipogenic cells differentiate into adipocytes in vitro. In some embodiments, the unengineered adipogenic cells differentiate into adipocytes in vivo. In some embodiments, the adipogenic cells, upon administration to a subject, provide a therapeutically effective amount of a protein. In some embodiments, the adipogenic cells express and/or secrete a therapeutically effective amount of a protein. Non-limiting examples of proteins expressed and/or secreted by unengineered adipogenic cells include phenylalanine hydroxylase (PAH); adiponectin; PEX5; ATP:cob(1)alamin adenosyl transferase (MMAB); 14-3-3 protein epsilon; 2-oxoisovalerate dehydrogenase subunit alpha, mitochondrial, BCKDHA; 2-Oxoisovalerate dehydrogenase subunit beta, mitochondrial, BCKDHB; 3-Hydroxyisobutyrate dehydrogenase (HIBADH); 3-Hydroxyisobutyryl-CoA deacylase (HIBCH); 3-Methylcrotonyl CoA carboxylase, MCCC1; 3-Methylcrotonyl CoA carboxylase, MCCC2; 4-Aminobutyrate-α-ketoglutarate aminotransferase (ABAT); 5-nucleotidase; 6-phosphogluconate dehydrogenase, decarboxylating; medium-chain acyl-CoA dehydrogenase, MCAD; short-chain acyl-CoA dehydrogenase, SCAD; very long-chain acyl-CoA dehydrogenase, VLCAD; Acetyl-CoA thiolase (acetyl-coenzyme A acetyltransferase), ACAT1; Acid ceramidase; Adenine phosphoribosyltransferase, APRT; Adenosine deaminase; Adipocyte enhancer-binding protein 1; Agrin; Aldehyde oxidase; Aldo-keto reductase family 1 member C2; Alkaline phosphatase, tissue-nonspecific isozyme; Alkyldihydroxyacetonephosphate synthase, AGPS; Alpha-2-macroglobulin; Alpha-enolase; Alpha-fetoprotein; Alpha-L-iduronidase, Alpha-N-acetylglucosaminidase; Alpha-N-acetylglucosaminidase 82 kDa form; Alpha-N-acetylglucosaminidase 77 kDa form; Aminoacylase-1; Angiotensinogen; Angiotensin-1; Angiotensin-2; Angiotensin-3; Angiotensin-4; Angiotensin 1-9; Angiotensin 1-7; Angiotensin 1-5; Angiotensin 1-4; Annexin A5; Adaptor Related Protein Complex 3 Subunit Beta 1, AP3B1; Apolipoprotein E; Argininosuccinate lyase, ASL; Argininosuccinate synthase; Argininosuccinic acid synthetase, ASS; Arylsulfatase A; Arylsulfatase A component B; Arylsulfatase A component C; Arylsulfatase B; aspartylglucosaminidase; ATP-binding cassette transporter, ABCD1; ATP-dependent RNA helicase, DDX3X; Endorepellin; Beta-2-microglobulin; Beta-galactosidase; Beta-hexosaminidase subunit alpha, HEXA; Beta-hexosaminidase subunit beta, HEXB; Bifunctional purine biosynthesis protein, PURH; Biglycan; Biotinidase; Biotinidase; Bone morphogenetic protein 1; Branching enzyme, GBE1; Calmodulin; Calreticulin; cAMP-dependent protein kinase catalytic subunit gamma; Cartilage oligomeric matrix protein; Cartilage-associated protein; Catalase; Catalase, CAT; Cathepsin A; Cathepsin B; Cathepsin D; Cathepsin F; Cathepsin K; Citrin, SLC25A13; Collagen alpha-1(I) chain; Collagen alpha-1(III) chain; Collagen alpha-1(IV) chain; Arresten; Collagen alpha-1(V) chain, Collagen alpha-1(XI) chain, Collagen alpha-1(XVIII) chain; Endostatin, Collagen alpha-2(I) chain; Collagen alpha-2(IV) chain; Canstatin; Collagen alpha-2(V) chain; Collagen alpha-2(VI) chain; Collagen alpha-3(VI) chain; Complement C1r subcomponent; Complement C1s subcomponent; Complement C3; Complement C4 beta chain; Complement factor D; Carnitine palmitoyltransferase 1A, CPT1A; Cystathionine β-synthase, CBS; Cystatin-C; Cystinosin, CTNS; Cytochrome c; Cytokine receptor-like factor 1; Cytoplasmic acetoacetyl-CoA thiolase, ACAT2; D-bifuncitonal enzyme, HSD17B4; Decorin; Dihydrolipoyl dehydrogenase, mitochondrial; Dihydroxyacetonephosphate acyltransferase, GNPAT; Dipeptidyl peptidase 1; Cathepsin C; EGF-containing fibulin-like extracellular matrix protein 1; EGF-containing fibulin-like extracellular matrix protein 2; Elastin; Elongation factor 2; Electron Transfer Flavoprotein Subunit Alpha, ETFA; Electron Transfer Flavoprotein Subunit Beta, ETFB; Electron transfer flavoprotein dehydrogenase, ETFDH; Extracellular matrix protein 1; Fibrillin-1; Fibrillin-2; Fibronectin; Fibulin-1; Fibulin-5; Formyl-Glycin generating enzyme, SUMF1; Fructose 1,6-biphosphatase, FBP1; Fumarylacetoacetase; Fumarylacetoacetate hydrolase domain-containing protein 2A, FAHD2A; Galactocerebrosidase; Galactokinase 1; Galactose-1-phosphate uridyl transferase, GALT; Ganglioside GM2 activator; Ganglioside GM2 activator isoform short; Gelsolin; GIcNAc phosphotransferase, GNPTA; Glucose-6-phosphate 1-dehydrogenase; Glucose-6-phosphate isomerase; Glucose-6-phosphate translocase, G6PT1; Glutaryl CoA dehydrogenase, GCDH; Glutathione peroxidase 3; Glutathione synthetase; Glycerol kinase; Glycerol-3-phosphate dehydrogenase [NAD(+)], cytoplasmic; Glycine cleavage enzyme system, AMT; Glycine cleavage enzyme system, GCSH; Glycogen debranching enzyme; 4-alpha-glucanotransferase; Amylo-alpha-1,6-glucosidase; Glycogen phosphorylase, liver form; Glypican-1; Glypican-6; Hydroxyacyl-CoA Dehydrogenase Trifunctional Multienzyme Complex Subunit Alpha, HADHA; Haptoglobin; Heparan N-sulfatase, N-sulfoglucosamine sulfohydrolase, SGSH; Heparan-alpha-glucosaminide N-acetyltransferase, HGSNAT; Hormone-sensitive lipase; Hydroxyacyl-coenzyme A dehydrogenase, mitochondrial; Hyperactivity of glutamate dehydrogenase, GLUD1; Hypoxanthine-guanine phosphoribosyltransferase, HPRT; Iduronate-2-sulfatase, IDS; Insulin-like growth factor-binding protein 7; Interstitial collagenase; Isovaleryl-CoA dehydrogenase; Keratin, type II cytoskeletal 1; Keratin, type II cytoskeletal 6B; L-lactate dehydrogenase A chain; L-lactate dehydrogenase B chain; Lactoylglutathione lyase; Laminin subunit alpha-2; Laminin subunit alpha-4; Laminin subunit beta-1; Laminin subunit beta-2; Laminin subunit gamma-1; Leptin; Lipoamide acyltransferase component of branched-chain alpha-keto acid dehydrogenase complex, mitochondrial, DBT; Lipoprotein lipase; Liver and muscle phosphorylase kinase, PHKB; Liver phosphorylase kinase, PHKG2; Lysosomal acid lipase/cholesteryl ester hydrolase; Lysosomal alpha-glucosidase; Lysosomal alpha-mannosidase; Lysosomal protective protein; CLN6 Transmembrane ER Protein, CLN6; CLN8 Transmembrane ER And ERGIC Protein, CLN8; Lysosomal transmembrane CLN3 protein, CLN3; Lysosomal transmembrane CLN5 protein, CLN5; Lysosome-associated membrane glycoprotein 2; Lysosomal trafficking regulator, LYST; Malonyl-CoA decarboxylase, MLYCD; Matrilin-3; Matrix Gla protein; Melanophilin, MLPH; Methionine synthase reductase, MTRR; Methylene tetrahydrofolate homocysteine methyltransferase, MTR; Methylenetetrahydrofolate reductase, MTHFR; Methylmalonic semialdehyde dehydrogenase, ALDH6A1; Methylmalonyl-CoA mutase; Mevalonate kinase; Mitochondrial branched-chain aminotransferase 2, BCAT2; Mitochondrial ornithine translocase, SLC25A15; Methylmalonic aciduria type A, MMAA; Molybdopterin synthase, Gephyrin, MOCS1A; Mucolipin-1, MCOLN1; Muscle phosphorylase kinase, PHKA1; Myosin Va, MYO5A; Myosin light chain 4; N-Acetylgalactosamine-6 Sulfatase, GALNS; N-acetylglucosamine-6-sulfatase; Nicotinamide N-methyltransferase; NPC intracellular cholesterol transporter 1, NPC1; Palmitoyl-protein thioesterase-1, PPT1; Palmitoyl-protein thioesterase, PPT2; Pentraxin-related protein, PTX3; Peptidyl-prolyl cis-trans isomerase, FKBP10; Peroxidasin homolog; Peroxin-1, 2, 3, 5, 6, 7, 10, 12, 13, 14, 26, Phosphoacetylglucosamine mutase; Phosphoglucomutase-1; Phosphoglycerate kinase 1; Phosphoglycerate mutase 1; Pigment epithelium-derived factor, PEDF; Plasma alpha-L-fucosidase; Plasma membrane carnitine transport, OCTN2; Plasma protease C1 inhibitor; Plasminogen activator inhibitor 1; Procollagen-lysine,2-oxoglutarate 5-dioxygenase 1; Propionyl-CoA carboxylase; Prosaposin; Proteoglycan 4; Proteoglycan 4 C-terminal part; Pyruvate carboxylase; Pyruvate dehydrogenase complex, DLAT; Pyruvate dehydrogenase complex, PDHB; Pyruvate dehydrogenase complex, PDHX; Pyruvate dehydrogenase complex, PDP1; Ras-related protein Rab-27A, RAB27A; Retinol-binding protein 4; Ribonuclease T2; Semaphorin-7A; Sepiapterin reductase; Serine protease, HTRA1; Serotransferrin; Serpin B6; Serum amyloid A-1 protein; Short branched-chain acyl-CoA dehydrogenase, ACADSB; Sialic acid synthase; Sialidase-1; Sialin (sialic acid transport), SLC17A5; Solute Carrier Family 22 Member 5, SLC22A5; SPARC-related modular calcium-binding protein 2; Spectrin alpha chain, non-erythrocytic 1; Sphingomyelin phosphodiesterase, SMPD1; Succinyl-CoA 3-oxoacid-CoA transferase, OXCT1; Sushi repeat-containing protein, SRPX2; Tafazzin; Tenascin; Thrombospondin-2; Transforming growth factor-beta-induced protein ig-h3; Transitional endoplasmic reticulum ATPase; Triosephosphate isomerase; Tripeptidyl-peptidase 1; Tumor necrosis factor receptor superfamily member 11B; Vascular endothelial growth factor C; Versican core protein; Vimentin; Vitamin K-dependent protein S; X-linked phosphorylase kinase, PHKA2; Xaa-Pro dipeptidase; α-Fucosidase, FUCA1; α-Galactosidase A, GLA; α-N-Acetylglucosaminidase, NAGA; β-Glucocerebrosidase (aka Glucosylceramidase); GBA, β-glucuronidase, GUSB; R-mannosidasen, VEGFA; VEGF165; FGF2; FGF4; PDGF-BB (platelet-derived growth factor); Ang1 (angiopoiten 1), TGFβ (transforming growth factor); LPA-producing enzyme (AXT); and phthalimide neovascularization factor (PNF1).

In some embodiments, the unengineered adipogenic cells express and/or secrete one or more of Lysosomal acid lipase, Adiponectin, Complement C3, Adiponcytes (whole cells), Adiponcytes (whole cells), Plasma protease C1 inhibitor, Propionyl-CoA carboxylase, Collagen alpha-1(V) chain, Hydroxyacyl-CoA Dehydrogenase Trifunctional Multienzyme Complex Subunit Alpha, Lysosomal acid lipase, Vitamin K-dependent protein S, Fumarylacetoacetate hydrolase domain-containing protein 2A, Adenine phosphoribosyltransferase, Citrin, Methylmalonic semialdehyde dehydrogenase, Succinyl-CoA 3-oxoacid-CoA transferase, Galactose-1-phosphate uridyl transferase, Glycerol kinase, Glycine cleavage enzyme system Protein H, Glutaryl CoA dehydrogenase, Molybdopterin synthase, and Peroxins.

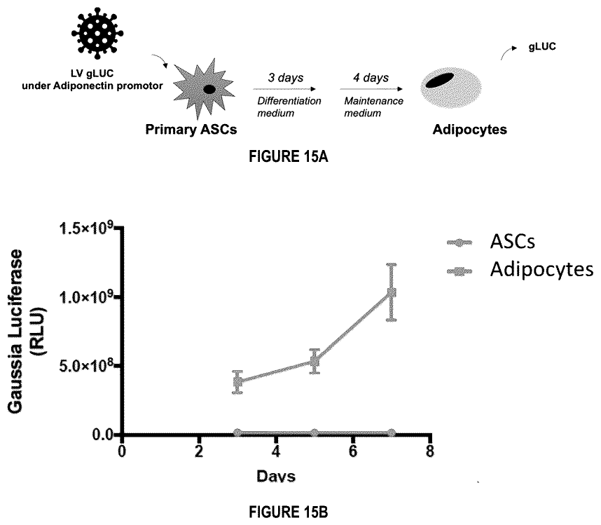

Non-limiting methods for generating adipogenic cells that express and/or secret any protein and/or molecule described herein include transfecting adipocyte progentiror cells (e.g. ASCs) with a lentivirus reporter vector expressing the protein and/or molecule, allowing the cells to differentiate, and collecting the engineered adipogenic cells. See, e.g., FIGS. 14A and 15A.

In some embodiments, the engineered adipogenic cells and/or the unengineered adipogenic cells express and/or secrete one or more of a therapeutically effective amount of a protein that regulates heme. Non-limiting examples of a protein that regulates heme include erythropoietin (EPO), EPOR, and GATA-1, epoetin alfa (e.g., Procrit and Epogen), epoetin beta (e.g., NeoRecormon), epoetin zeta (e.g., Silapo and Retacrit), darbepoetin alfa (e.g., Aranesp), and methoxy polyethylene glycol-epoetin beta (e.g., Mircera). In some embodimetns, the protein that reguates heme also regulates EPO, including, but not limited to, Hypoxia Inducible Factors (HIFs), which regulate EPO which regulates heme-containing cells.

In some embodiments, the adipogenic cells comprise a combination of engineered adipogenic cells and unengineered adipogenic cells. In some embodiments, the ratio of engineered adipogenic cells to unengineered adipogenic cells is between about 1:99 and about 99:1. In some embodiments, the ratio of engineered adipogenic cells to unengineered adipogenic cells is between about 1:50 and about 50:1. In some embodiments, the ratio of engineered adipogenic cells to unengineered adipogenic cells is between about 1:25 and about 25:1. In some embodiments, the ratio of engineered adipogenic cells to unengineered adipogenic cells is between about 1:10 and about 10:1. In some embodiments, the ratio of engineered adipogenic cells to unengineered adipogenic cells is between about 1:5 and about 5:1. In some embodiments, the ratio of engineered adipogenic cells to unengineered adipogenic cells is between about 1:2 and about 2:1. In some embodiments, the ratio of engineered adipogenic cells to unengineered adipogenic cells is about 1:1.

In some embodiments, the adipogenic cells comprise a heterologous nucleic acid. Examples of heterologous nucleic acids include, but are not limited to, DNA or RNA that encodes a gene product or gene product(s) of interest, introduced, for example, for purposes of production of an encoded protein. In some embodiments, the heterologous nucleic acid comprises an adipocyte-specific promoter. Non-limiting examples of adipocyte-specific promoters include an adiponectin promoter and an aP2/FABP4 promoter. In some embodiments, the adipocyte-specific promoter comprises a minimal proximal promoter sequence. In some embodiments, the adipocyte-specific promoter optionally further comprises one or more of a distal enhancer sequence and additional transcription factor binding site. In some embodiments, the transcription factor binding site is a C/EBPα binding site. In some embodiments, the adipocyte specific promoter is an adiponectin promoter. In some embodiments, the adiponectin promotor is a human adiponectin promoter. In some embodiments, the adipocyte specific promoter is in operative association with one or more therapeutic proteins.

In some embodiments, the adipocyte-specific promoter is selected from adiponectin or ap2/FABP4. In some embodiments, the adipocyte-specific promoter is selected from CFD, FABP4, PLIN2, PLIN4, LEP, LIPE, PPARγ, Resistin, IsG12b, and ACVR1C.

In some embodiments, the promoter is a non-adipocyte-specific promoter and/or is a partially adiopocyte-specific promoter. In some embodiments, the non-adipocyte-specific promoter and/or partially adiopocyte-specific promoter is selected from DCN, ADH1B, and HAS1.

In some embodiments, the promoter is a constitutive promoter. In some embodiments, constitutive promoters are useful for transgene expression. In some embodiments, the constitutive promoter is selected from EF1a, CMV, and CAG.

In some embodiments, the therapeutic protein has one or more of antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Examples of therapeutic proteins include, but are not limited to, an enzyme replacement protein, a protein for supplementation, a protein vaccination, antigens (e.g. tumor antigens, viral, bacterial), hormones, cytokines, antibodies, immunotherapy (e.g. cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

In some embodiments, the heterologous nucleic acid comprises one or more RNA expression sequences, each of which may encode a polypeptide. In some embodiments, the polypeptide is produced in substantial amounts. As such, the polypeptide may be any proteinaceous molecule that can be produced. In some embodiments, a polypeptide can be a polypeptide that can be secreted from a cell, or localized to the cytoplasm, nucleus or membrane compartment of a cell. Examples of polypeptides include, but are not limited to, at least a portion of a viral envelope protein, metabolic regulatory enzymes (e.g., that regulate lipid or steroid production), an antigen, a toleragen, a cytokine, a toxin, enzymes whose absence is associated with a disease, and polypeptides that are not active in an animal until cleaved (e.g., in the gut of an animal), and a hormone.

In some embodiments, proteins that can be expressed from the heterologous nucleic acid include a human protein, for instance, receptor binding protein, hormone, growth factor, growth factor receptor modulator, and regenerative protein (e.g., proteins implicated in proliferation and differentiation, e.g., therapeutic protein, for wound healing). In some embodiments, exemplary proteins that can be expressed from the heterologous nucleic acid include EGF (epithelial growth factor). In some embodiments, exemplary proteins that can be expressed from the heterologous nucleic acid include enzymes, for instance, oxidoreductase enzymes, metabolic enzymes, mitochondrial enzymes, oxygenases, dehydrogenases, ATP-independent enzyme, and desaturases. In some embodiments, exemplary proteins that can be expressed from the heterologous nucleic acid include an intracellular protein or cytosolic protein. In some embodiments, the protein is NanoLuc® luciferase (nLuc). In some embodiments, the exemplary proteins that can be expressed from heterologous nucleic acid include a secretary protein, for instance, a secretary enzyme. In some cases, the heterologous nucleic acid expresses a secretary protein that can have a short half-life therapeutic in the blood, or can be a protein with a subcellular localization signal, or protein with secretory signal peptide. In some embodiments, the heterologous nucleic acid expresses a gaussia Luciferase (gLuc). In some cases, the heterologous nucleic acid expresses a non-human protein, for instance, a fluorescent protein, an energy-transfer acceptor, or a protein-tag like Flag, Myc, or His. In some embodiments, exemplary proteins that can be expressed from the heterologous expresses includes a GFP. In some embodiments, the heterologous nucleic acid expresses tagged proteins, e.g., fusion proteins or engineered proteins containing a protein tag, e.g., chitin binding protein (CBP), maltose binding protein (MBP), Fc tag, glutathione-S-transferase (GST), AviTag (GLNDIFEAQKIEWHE; SEQ ID NO: 14), Calmodulin-tag (KRRWKKNFIAVSAANRFKKISSSGAL; SEQ ID NO: 15); polyglutamate tag (EEEEEE; SEQ ID NO: 16); E-tag (GAPVPYPDPLEPR; SEQ ID NO: 17); FLAG-tag (DYKDDDDK; SEQ ID NO: 18), HA-tag (YPYDVPDYA; SEQ ID NO: 19); His-tag (HHHHHH; SEQ ID NO: 20); Myc-tag (EQKLISEEDL; SEQ ID NO: 21); NE-tag (TKENPRSNQEESYDDNES; SEQ ID NO: 22); S-tag (KETAAAKFERQHMDS; SEQ ID NO: 23); SBP-tag (MDEKTTGWRGGHVVEGLAGELEQLRARLEHHPQGQREP; SEQ ID NO: 24); Softag 1 (SLAELLNAGLGGS; SEQ ID NO: 25); Softag 3 (TQDPSRVG; SEQ ID NO: 26); Spot-tag (PDRVRAVSHWSS; SEQ ID NO: 27); Strep-tag (Strep-tag II: WSHPQFEK; SEQ ID NO: 28); TC tag (CCPGCC; SEQ ID NO: 29); Ty tag (EVHTNQDPLD; SEQ ID NO: 30); V5 tag (GKPIPNPLLGLDST; SEQ ID NO: 31); VSV-tag (YTDIEMNRLGK; SEQ ID NO: 32); or Xpress tag (DLYDDDDK; SEQ ID NO: 33).

In some embodiments, the heterologous nucleic acid expresses an antibody, e.g., an antibody fragment, or a portion thereof, such as an antigen-binding fragment of an antibody, including scFvs and conjugates or multimers thereof. In some embodiments, the antibody expressed by the adipogenic cells can be of any isotype, such as IgA, IgD, IgE, IgG, IgM. In some embodiments, the heterologous nucleic acid expresses a portion of an antibody, such as a light chain, a heavy chain, a Fc fragment, a CDR (complementary determining region), a Fv fragment, or a Fab fragment, a further portion thereof. In some embodiments, the heterologous nucleic acid expresses one or more portions of an antibody. For instance, the heterologous nucleic acid can comprise more than one expression sequence, each of which expresses a portion of an antibody, and the sum of which can constitute the antibody. In some cases, the heterologous nucleic acid comprises one expression sequence coding for the heavy chain of an antibody, and another expression sequence coding for the light chain of the antibody. In some cases, when the heterologous nucleic acid expresses a light chain and heavy chain can be subject to appropriate modification, folding, or other post-translation modification to form a functional antibody.

In embodiments, the adipogenc cells of the dislosure comprise a modification that modulates cell death. In embodiments, the modification is or comprises a suicide switch. Non-limiting examples of suicide switches include herpes simplex virus thymidine kinase (HSV-tk), caspase 9 (iCasp9), CD20/eGFRt expression, and HLA-targeting antibodies. In some embodiments, the suicide switch is a drug-induced suicide switch, such as by way of example, HSV-tk, iCasp9, and CD20/eGFRt expression. In some embodiments, the suicide switch is HSV-tk. In some embodiments, HSV-tk is used in combination with ganciclovir (GCV). See, for example, Moolten and Wells, J Natl Cancer Inst. 82:297-300 (1990) and Sangro et al., Cancer Gene Ther. 17:837-843 (2010), both of which are incorporated by reference herein in their entireties. HSV-tk phosphorylates specific nucleoside analogues, such as GCV, forming a toxic GCV-triphosphate compound that competes with triphosphate as a substrate incorporated into DNA via the action of DNA polymerase, leading to the inhibition of DNA synthesis and subsequent cellular death. In some embodiments, the suicide switch is or comprises a capsase, or a modified version thereof, e.g. iCasp9. In some embodiments, iCasp9 is used in combination with a chemical inducer of dimerization (CID). Non-limiting examples of CIDs include rimiducid (AP1903) and rapamycin and/or a rapalog. In embodiments, iCasp9 contains a modified human caspase 9 fused to the human FK506 binding protein (FKBP), e.g. FKBP12, and conditional administration of a CID forms dimerization and activates the downstream caspase molecules, resulting in apoptosis of cells expressing the fusion protein. See, for example, Gargett and Brown, Front. Pharmacol. 5:235 (2014), which is incorporated by reference herein in its entirety. In some embodiments, the suicide switch is or comprises a FKBP, e.g., FKBP12, region and is capable of binding or interacting with a CID. In some embodiments, the suicide switch is CD20/eGFRt. In some embodiments, the adipogenc cells express CD20/eGFRt and this suicide switch is used in combination with an antibody targeting modified adipogenic cells. In some embodiments, the suicide switch is HLA targeting antibodies. In a non-limiting example, the HLA targeting antibodies depend on the donor. In another non-limiting example, the suicide switch is or comprises RQR8. In some embodiments, the suicide switch is or comprises truncated EGF receptor (EGFRt). In some embodiments, modification that modulates cell death includes removal of one or more engraftments of adipogenc cells of the disclosure.

### Compositions

In one aspect, the present invention includes a composition comprising adipogenic cells described herein. In some embodiments, the composition comprises a therapeutically effective amount of the adipogenic cells.

In some embodiments, the composition is allogenic or includes allogenic cells.

In some embodiments, the composition is non-immunogenic. For example, the composition does not result in an inflammatory reaction upon administration. In some embodiments, the adipogenic cells are non-immunogenic. In some embodiments, upon administration a subject, the composition, optionally the adipogenic cells therein, elicits less than about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% increase in an inflammatory cytokine, such as TNF-alpha, IL-2, or IFN-gamma, or any combination thereof. In some embodiments, the composition and/or the adipogenic cells do not express and/or secrete proteins that are associated with an immune response, or express and/or secrete level of proteins associated with an immune response at a reduced level such that the subject does not exhibit an immune response when administered the composition and/or the adipogenic cells.

In some embodiments, upon administration a subject, the composition elicits an increase of about 5%, about 10%, about 15%, about 20%, about 25%, about 30%, about 35%, about 40%, about 45%, about 50%, about 60%, about 70%, about 80%, about 90%, about 100%, about 150%, about 200%, about 250%, about 300%, about 350%, or about 400% or more of one or more cytokines selected from IDO, HLA-G, HGF, PGE2, TGFbeta, and IL-6, or any combination thereof, upon administration to a subject.

In some embodiments, the composition is long-acting. In embodiments, a long-acting composition, such as a long-acting composition of adipogenic cells described herein, is capable of providing therapeutic effect, such as protein, lipid, or hormone secretion at therapeutically-effective levels, for extended periods, such as, in some embodiments, at least about 1, about 2, about 3, about 4, about 5, about 6, about 7, about 8, about 9, about 10, about 11, about 12, about 15, about 18, about 21, or about 24 months to about 2.5, about 3, about 3.5, about 4, about 4.5, about 5, about 5.5, about 6, about 6.5, about 7, about 7.5, about 8, about 8.5, about 9, about 9.5, or about 10 years. In embodiments, a long-acting composition, such as a long-acting composition of adipogenic cells described herein, is capable of providing therapeutic effect, such as protein, lipid, or hormone secretion at therapeutically-effective levels, for extended periods, such as, in some embodiments, at least about 1, about 2, about 3, about 4, about 5, about 6, about 7, about 8, about 9, about 10, about 11, about 12, about 15, about 18, about 21, about 24 months, about 2.5, about 3, about 3.5, about 4, about 4.5, about 5, about 5.5, about 6, about 6.5, about 7, about 7.5, about 8, about 8.5, about 9, about 9.5, or about 10 years.

In one aspect, the present invention includes compositions comprising adipogenic cells described herein. In some embodiments, the composition is an allogenic, non-immunogenic, long-acting composition comprising a therapeutically effective amount of substantially pure adipogenic cells. In some embodiments, the composition is an autologous, non-immunogenic, long-acting composition comprising a therapeutically effective amount of substantially pure adipogenic cells, wherein the adipogenic cells comprise one or more heterologous nucleic acid. In some embodiments, the composition is capable of treating, preventing, or ameliorating a disease or disorder in a subject in need thereof.

In some embodiments, the composition comprises about 50,000 to about 6,000,000,000 adipogenic cells, optionally selected from one or more of adipocytes and adipocyte precursor cells (such as adipogenic stem cells (ASCs), and CD34⁺ cells) (e.g. about 50,000 to about 5,000,000,000, about 50,000 to about 4,000,000,000, about 50,000 to about 3,000,000,000, about 50,000 to about 2,000,000,000, about 50,000 to about 1,000,000,000, about 50,000 to about 500,000,000, about 50,000 to about 100,000,000, about 50,000 to about 10,000,000, about 50,000 to about 1,000,000 cells, optionally selected from one or more of adipocytes and adipocyte precursor cells (such as adipogenic stem cells (ASCs), and CD34⁺ cells)).

In some embodiments, the adipocytes are present in the composition at a concentration of about 70,000,000 cells/mL to about 3,000,000 cells/mL. In some embodiments, the adipocytes are present in the composition at a concentration of about 50,000,000 cells/mL to about 10,000,000 cells/mL. In some embodiments, the adipocytes are present in the composition at a concentration of about 40,000,000 cells/mL to about 20,000,000 cells/mL. In some embodiments, the adipocytes are present in the composition at a concentration of about 38,000,000 cells/mL. In some embodiments, the adipocytes are present in the composition at a concentration of about 30,000,000 cells/mL. In some embodiments, the adipocytes are present in the composition at a concentration of about 5,000,000 cells/mL.

In some embodiments, the ASCs are present in the composition at a concentration of about 0.1 million cells/mL to about 100 million cells/mL (e.g. about 0.1 million cells/mL to about 10 million cells/mL, about 0.1 million cells/mL to about 1 million cells/mL, or about 0.1 million cells/mL to about 0.5 million cells/mL). In some embodiments, the ASCs are present in the composition at a concentration of about 5 million cells/mL.

In some embodiments, the composition comprises about 1 million to about 750 million ASCs. In some embodiments, the composition comprises about 120 million ASCs. In some embodiments, the composition comprises about 4×10⁶ ASCs.

In some embodiments, the ASCs are present in the composition at a concentration of about 250,000 cells/kg to about 4 million cells/kg.

In some embodiments, the composition comprises about 0.2×10⁶ to about 0.8×10⁶ CD34⁺ cells.

In some embodiments, the composition is substantially free of one or more bacteria, virus, fungus, and pyrogen, and in more particular embodiments is substantially free of all of the foregoing.

### Pharmaceutical Compositions and Formulations

In one aspect, the composition is a pharmaceutical composition. In some embodiments, the pharmaceutical compositions of the present invention are formulated to provide a therapeutically effective amount of adipogenic cells, as described herein, as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any composition disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In some embodiments, the composition comprises an excipient or carrier. In some embodiments, the diluent is a pharmaceutically acceptable excipient or carrier.

In some embodiments, the composition comprises a diluent. In some embodiments, the diluent is a pharmaceutically acceptable diluent. Non-limiting example of diluents include liquid diluents such as water, ethanol, propylene glycol, glycerin and various combinations thereof, and inert solid diluents such as calcium carbonate, calcium phosphate or kaolin. In some embodiments, the diluent comprises one or more of saline, phosphate buffered saline, Dulbecco's Modified Eagle Medium DMEM, alpha modified Minimal Essential Medium (alpha MEM), Roswell Park Memorial Institute Media 1640 (RPMI Media 1640), HBSS, human albumin, and Ringer's solution and the like, or any combination thereof.

In some embodiments, the composition further comprises a therapeutically effective amount of one or more of heparin, FBS, human albumin, bFGF, PPAR-y agonists, insulin, and a Rho kinase inhibitor, or any combination thereof. Non-limiting examples of PPAR-y agonists include Rosiglitazone, GW-9662, Tesaglitazar, GW 1929 hydrochloride, Ciglitazone, nTZDpa, Troglitazone, Genistein, Telmisartan, Edaglitazone, 15-deoxy-Δ-12,14-Prostaglandin J2, and Pioglitazone hydrochloride. Non-limiting examples of Rho kinase inhibitors include Fasudil, Y27632, Rhopressa, and Netarsudil.

In some embodiments, the diluent further comprises of one or more of heparin, FBS, human albumin, bFGF, PPAR-y agonists, insulin, and a Rho kinase inhibitor, or any combination thereof.

In embodiments, the compositions, e.g., pharmaceutical compositions, disclosed herein are suspended in a saline buffer (including, without limitation TBS, PBS, and the like).

The present technology includes the disclosed adipogenic cells in various formulations of pharmaceutical compositions. Any adipogenic cells disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the adipogenic cells can also include a solubilizing agent. Also, the agents can be delivered with a suitable vehicle or delivery device as known in the art.

In some embodiments, the composition comprises a scaffold. In some embodiments, the scaffold comprises biomaterials. In a non-limiting example, the three-dimensional biomaterials include adipocytic cells embedded in an extracellular matrix attached to, or dispersed within, or trapped within the scaffold. In some embodiments, the biomaterials are biodegradeable and/or synthetic.

In some embodiments, the scaffold comprises biodegradable biomaterials. Non-limiting examples of biodegradable biomaterials include fibrin, collagen, elastin, gelatin, vitronectin, fibronectin, laminin, reconstituted basement membrane matrix, starch, dextran, alginate, hyaluron, chitin, chitosan, agarose, sugars, hyaluronic acid, poly (lactic acid), poly (glycolic acid), polyethylene glycol, decellularized tissue, self-assembling peptides, polypeptides, glycosaminoglycans, derivatives and mixtures thereof. Other useful biodegradable polymers or polymer species include, but are not limited to, polydioxanone, polycarbonate, polyoxalate, poly (α-ester), polyanhydride, polyacetate, polycaprolactone, poly (ortho Esters), polyamino acids, polyamides, and mixtures and copolymers thereof, L-lactic acid and D-lactic acid stereopolymers, copolymers of bis (para-carboxyphenoxy) propanoic acid and sebacic acid, sebacic acid copolymers, caprolactone Copolymer, poly (lactic acid)/poly (glycolic acid)/polyethylene glycol copolymer, polyurethane and poly (lactic acid) copolymer, polyurethane and poly (lactic acid) copolymer, α-amino acid copolymer, α-amino acid and caproic acid copolymer, A-benzylglutamate and polyethylene glycol copolymers, succinate and poly (glycol) copolymers, polyphosphazenes, polyhydroxy-alkanoates and mixtures thereof. Binary and ternary systems are also contemplated. In some embodiments, the scaffold comprises one or more of collagen, various proteoglycans, alginate-based substrates and chitosan. In some embodiments, the scaffold comprises one or more of a hydrogel, silk, Matrigel, acellular and/or decellarized scaffolds, poly-ε-caprolactone scaffolds, resorbable scaffolds, and nanofiber-hydrogel composite.

In some embodiments, the scaffold comprises synthetic biomaterials. Non-limiting examples of synthetic biomaterials include lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters.

In some embodiments, the scaffold comprises one or more of a hydrogel, a matrigel, alginates, collagens, chitosans, PGAs, PLAs, and PGA/PLA copolymers, biodegradable biomaterials (e.g. collagen, proteoglycans, alginate-based substrates, chitosan) or any combination thereof.

For additional examples of formulations, see US 20160324982, US 20180077922, and KR 20160147929, all of which are incorporated by reference herein in their entireties.

In some embodiments, the composition further comprises a therapeutically effective amount of one or more additional therapeutic agents. In some embodiments, the additional therapeutic agent is one or more of an analgesic and an anti-infective agent. For example, a composition may contain an analgesic, to aid in treating inflammation or pain at the site of the fistula, or an anti-infective agent to prevent infection of the site treated with the composition. Non-limiting examples of additional therapeutic agents include analgesics, such as nonsteroidal anti-inflammatory drugs, opiate agonists and salicylates; anti-infective agents, such as antihelmintics, antianaerobics, antibiotics, aminoglycoside antibiotics, antifungal antibiotics, cephalosporin antibiotics, macrolide antibiotics, miscellaneous B-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents, minerals; and vitamins, such a s vitamin A, vitamin B, vitamin C, vitamin D, vitamin E, and vitamin K.

Additional non-limiting examples of useful therapeutic agents from the above categories include: (1) analgesics in general, such as lidocaine or derivatives thereof, and nonsteroidal anti-inflammatory drugs (NSAIDs) analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) Hi-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous B-lactam antibiotic anti-infectives, such as aztreonam and imipenem; (10) penicillin antibiotic anti-infectives, such a s nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) uricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H2-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketorlac, and naproxen; (36) minerals, such as iron, calcium, and magnesium; (37) vitamin B compounds, such as cyanocobalamin (vitamin B12) and niacin (vitamin B3); (38) vitamin C compounds, such as ascorbic acid; and (39) vitamin D compounds, such as calcitriol.

In some embodiments, the therapeutic agent may be a growth factor or other molecule that affects cell differentiation and/or proliferation. Growth factors that induce final differentiation states are well-known in the art, and may be selected from any such factor that has been shown to induce a final differentiation state. Growth factors for use in methods described herein may, in certain embodiments, be variants or fragments of a naturally-occurring growth factor. For example, a variant may be generated by making conservative amino acid changes and testing the resulting variant in one of the functional assays described above or another functional assay known in the art. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Non-limiting examples of conservative amino acids substitution groups include valine-leucine-isoleucine, phenylalanine-tyrosine, lysine-arginine, alanine-valine, and asparagine-glutamine.

As those skilled in the art will appreciate, variants or fragments of polypeptide growth factors can be generated using conventional techniques, such as mutagenesis, including creating discrete point mutation(s), or by truncation. For instance, mutation can give rise to variants which retain substantially the same, or merely a subset, of the biological activity of a polypeptide growth factor from which it was derived.

The pharmaceutical compositions comprising the adipogenic cells described herein may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any adipogenic cells disclosed herein are formulated in accordance with routine procedures as a pharmaceutical composition adapted for a mode of administration disclosed herein.

### Methods of Treatment

In one aspect, the present invention includes methods for treating, preventing, or ameliorating a disease or disorder in a subject in need thereof, comprising administering a composition comprising an effective amount of adipogenic cells of the present invention to the subject. In some embodiments, the subject has the disease or disorder. In some embodiments, the subject is suspected of having the disease or disorder. In some embodiments, the subject has an elevated risk for the disease or disorder. In some embodiments, the subject is suspected of having an elevated risk for the disease or disorder. In some embodiments, the adipogenic cells are substantially pure.

In some embodiments, the disease or disorder is associated with abnormal protein production. In some embodiments, the disease or disorder is associated with complete deficiency of a protein.

In some embodiments, the method comprises administering a composition comprising unengineered or non-transformed adipogenic cells. Non-limiting examples of diseases or disorders that can be treated, prevented, or ameliorated by administering unengineered or non-transformed adipogenic cells include Lysosomal storage disorders, Metabolic disorders, Complement deficiencies, Adipocyte disorders, Endocrine disorders, Vascular diseases, Branched-chain amino acid metabolism disorders, Connective tissue disorders, Fatty acid transport and mitochrondrial oxidation disorders, Genetic dyslipidemias, Hematological disorders, Phenylalanine and tyrosine metabolism disorders, Purine metabolism disorders, Urea cycle disorders, Beta-amino acid and gamma-amino acid disorders, Ketone metabolism disorders, Galactosemia, Glycerol Metabolism Disorders, Glycine Metabolism Disorders, Lysine Metabolism Disorders, Methionine and Sulfur Metabolism Disorders, and Peroxisome biogenesis and very long chain fatty acid metabolism disorders. Table 1 below shows non-limiting examples of classes of diseases and disorders and example indications that can be treated, prevented, or ameliorated using unengineered adipogenic cells of the present invention.

TABLE 1
Illustrative diseases or disorders against which unengineered adipogenic cells are useful
Unengineered Adipogenic Cells

| | | Protein and/or other |
|---|---|---|
| Class of disease | Example | molecule provided by |
| or disorders | indications | differentiated adipocytes |
| Lysosomal storage disorders | Wolman | Lysosomal acid lipase |
| Metabolic disorders | Obesity | Adiponectin |
| Complement deficiencies | C3 deficiency | Complement C3 |
| Adipocyte disorders | Familial lipodystrophy | Adiponcytes (whole cells) |
| Endocrine disorders | Cachexia | Adiponcytes (whole cells) |
| Vascular diseases | Hereditary angioedema | Plasma protease C1 inhibitor |
| Branched-chain amino acid | Propionic acidemia Type 1 | Propionyl-CoA carboxylase |
| metabolism disorders | | |
| Connective tissue disorders | Ehlers-Danlos syndrome | Collagen alpha-1(V) chain |
| Fatty acid transport and | long-chain 3-hydroxy acyl- | Hydroxyacyl-CoA Dehydrogenase |
| mitochrondrial oxidation | CoA dehydrogenase | Trifunctional Multienzyme Complex |
| disorders | deficiency | Subunit Alpha |
| Genetic dyslipidemias | Familial LPL deficiency | Lysosomal acid lipase |
| Hematological disorders | Protein S deficiency | Vitamin K-dependent protein S |
| Phenylalanine and tyrosine | Tyrosinemia type I | Fumarylacetoacetate hydrolase |
| metabolism disorders | | domain-containing protein 2A |
| Purine metabolism | Adenine | Adenin |
| disorders | phosphoribosyltransferase | phosphoribosyltransferase |
| | deficiency | |
| Urea cycle disorders | Citrullinemia type I | Citrin |
| Beta-amino acid and | Methylmalonic | Methylmalonic |
| gamma-amino acid disorders | semialdehyde | semialdehyde |
| | dehydrogenase deficiency | dehydrogenase |
| Ketone metabolism | Succinyl-CoA 3-oxoacid- | Succinyl-CoA 3-oxoacid- |
| disorders | CoA transferase | CoA transferase |
| | deficiency | |
| Galactosemia | Galactose-1-phosphate | Galactose-1-phosphate |
| | uridyl transferase | uridyl |
| | deficiency | transferase |
| Glycerol Metabolism | Glycerol kinase | Glycerol kinase |
| Disorders | deficiency | |
| Glycine Metabolism | Nonketotic | Glycine cleavage enzyme |
| Disorders | hyperglycinemia | system Protein H |
| Lysine Metabolism | Glutaric acidemia | Glutaryl CoA |
| Disorders | type I | dehydrogenase |
| Methionine and Sulfur | Molybdenum cofactor | Molybdopterin |
| Metabolism Disorders | defect | synthase |
| Peroxisome biogenesis & | Zellweger syndrome | Peroxins |
| very long chain fatty acid | | |
| metabolism disorders | | |

In some embodiments, the disease or disorder is selected from Wolman disease, Obesity, C3 deficiency, Familial lipodystrophy, Cachexia, Hereditary angioedema, Propionic acidemia Type 1, Ehlers-Danlos syndrome, long-chain 3-hydroxy acyl-CoA dehydrogenase deficiency, Familial LPL deficiency, Protein S deficiency, Tyrosinemia type I, Adenine phosphoribosyltransferase deficiency, Citrullinemia type I, Methylmalonic semialdehyde dehydrogenase deficiency, Succinyl-CoA 3-oxoacid-CoA transferase deficiency, Galactose-1-phosphate uridyl transferase deficiency, Glycerol kinase deficiency, Nonketotic hyperglycinemia, Glutaric acidemia type I, Molybdenum cofactor defect, and Zellweger syndrome.

In some embodiments, the method comprises administering a composition comprising engineered or transformed adipogenic cells. In some embodiments, the adipogenic cells comprise a heterologous nucleic acid. In some embodiments, the heterologous nucleic acid comprises a therapeutic transgene. Non-limiting examples of diseases or disorders that can be treated, prevented, or ameliorated by administering engineered or transformed adipogenic cells include Lysosomal storage disorders, Metabolic disorders, Hematological disorders, Bone and connective tissue disorders, Endocrine disorders, Inflammatory disorders, Monogenic disorders, Cancer, Cardiovascular disorders, Branched-chain amino acid metabolism disorders, Fatty acid transport and mitochrondrial oxidation disorders, Genetic dyslipidemias, Phenylalanine and tyrosine metabolism disorders, Purine metabolism disorders, Urea cycle disorders, Ketone metabolism disorders, Glycine Metabolism Disorders, Lysine Metabolism Disorders, Methionine and Sulfur Metabolism Disorders, Peroxisome biogenesis and very long chain fatty acid metabolism disorders, and other protein deficiency disorders. Table 2 below shows non-limiting examples of classes of diseases and disorders and example indications that can be treated, prevented, or ameliorated using engineered adipogenic cells of the present invention.

TABLE 2
Illustrative diseases or disorders against which engineered adipogenic cells are useful
Engineered adipogenic cells

| | | Therapeutic protein |
|---|---|---|
| Classes of | Example | engineered to be provided |
| disorders | indications | by differentiated adipocytes |
| Lysosomal storage | Cystinosis | Cystinosin |
| disorders | | |
| Metabolic disorders | T2D | GLP-1 |
| Hematological disorders | Hemophilia A, B | Factor VIII, Factor IX |
| Bone and connective | Stickler syndrome | COL2A1 |
| tissue disorders | | |
| Endocrine disorders | Osteoporosis | Parathyroid hormone (1-84) |
| Inflammatory disorders | Rheumatoid Arthritis | alkaline phosphatase |
| Monogenic disorders | A1AT deficiency | alpha-1 antitrypsin |
| Cancer | Breast cancer | Trastuzumab |
| Cardiovascular disorders | Atherosclerosis | Apolipoprotein A1 |
| Branched-chain amino acid | Isobutyryl-CoA | Isobutyryl-CoA |
| metabolism disorders | dehydrogenase deficiency | dehydrogenase |
| Fatty acid transport and | carnitine-acylcarnitine | SLC25A20 |
| mitochrondrial oxidation disorders | translocase deficiency | |
| Genetic dyslipidemias | Sitosterolemia | ATP-binding cassette sub- |
| | | family G member 5, ABCG5 |
| Phenylalanine and tyrosine | Phenylketonuria | Phenylalanine hydroxylase |
| metabolism disorders | | |
| Purine metabolism disorders | Hereditary xanthinuria | Xanthine dehydrogenase |
| Urea cycle disorders | Ornithine- | Ornithine- |
| | transcarbamoylase | transcarbamoylase |
| | deficiency | |
| Ketone metabolism | 3-Hydroxy-3-methylglutaryl- | 3-Hydroxy-3-methylglutaryl- |
| disorders | CoA synthase deficiency | CoA synthase |
| Glycine Metabolism | Nonketotic hyperglycinemia | Glycine cleavage |
| Disorders | | system P protein |
| Lysine Metabolism | Hyperlysinemia | Lysine:α-ketoglutarate |
| Disorders | | reductase |
| Methionine and Sulfur | Homocystinuria | Cystathionine β-synthase |
| Metabolism Disorders | | |
| Peroxisome biogenesis & very | Refsum disease | Phytanoyl-CoA hydroxylase |
| long chain fatty acid | | |
| metabolism isorders | | |
| Other protein deficiency | Growth Failure In Children | human growth hormone |
| disorders | With Kidney Disease | (somatotropin) |

In some embodiments, the disease or disorder is selected from is selected from Cystinosis, T2D, Hemophilia A or B, Stickler syndrome, Osteoporosis, Rheumatoid Arthritis, A1AT deficiency, Breast cancer, Atherosclerosis, Isobutyryl-CoA dehydrogenase deficiency, carnitine-acylcarnitine translocase deficiency, Sitosterolemia, Phenylketonuria, Hereditary xanthinuria, Ornithine-transcarbamoylase deficiency, 3-Hydroxy-3-methylglutaryl-CoA synthase deficiency, Nonketotic hyperglycinemia Hyperlysinemia, Homocystinuria, Refsum disease, and growth failure in children with kidney disease.

In some embodiments, the disease or disorder is hyperphenylalaninemia (HPA). In some embodiments, the disease or disorder is anemia.

In one aspect, the present invention includes methods for increasing red blood cell production in a subject in need thereof, comprising administering a composition comprising an effective amount of adipogenic cells of the present invention to the subject. In some embodiments, the method comprises administering adipogenic cells that express and/or secrete a heme factor

In one aspect, the composition of the invention is administered to a subject in need thereof for the treatment, prevention, or amelioration of a disease or disorder. In some embodiments, the composition can be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant. In some embodiments, the composition is administered to the subject in a single administration. In a non-limiting example, a single administration includes administration at one single site or at multiple sites. In some embodiments, the composition is administered to the subject in multiple administrations. In a non-limiting example, multiple administrations include repeated administration at one single site or at multiple sites. In some embodiments, the composition is capable of treating, preventing, or ameliorating a disease or disorder in the subject when administered in a single administration. In some embodiments, the composition is capable of treating, preventing, or ameliorating a disease or disorder in the subject when administered in multiple administrations. In some embodiments, the composition is formulated for administration by a route selected from subcutaneous, intradermal, intramuscular, intracranial, intraocular, intravenous, and fat pad. In some embodiments, the composition is administered subcutaneously, intradermally, intramuscularly, intracranially, intraocularly, intravenously, and into a fat pad. In some embodiments, the composition is administered by subcutaneous injection. In some embodiments, the adipogenic cells are transplanted into the subject.

The dosage of any adipogenic cells disclosed herein as well as the dosing schedule can depend on various parameters and factors, including, but not limited to, the specific adipogenic cells, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

Adipogenic cells disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any adipogenic cells disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific composition of the invention employed. Any adipogenic cells disclosed herein can be administered in a single daily dose, or the total daily dosage can be administered in divided doses of two, three or four times daily. Furthermore, any adipogenic cells disclosed herein can be administered continuously rather than intermittently throughout the dosage regimen.

In some embodiments, a combined remission or clinical remission of the disease or disorder is achieved within 24 weeks, 18 weeks, 12 weeks, 8 weeks, or 6 weeks from administration of the composition.

In some embodiments, the adipogenic cells are CD34⁺ cells and the disease or disorder is selected from Wolman disease, Obesity, C3 deficiency, Familial lipodystrophy, Cachexia, Hereditary angioedema, Propionic acidemia Type 1, Ehlers-Danlos syndrome, long-chain 3-hydroxy acyl-CoA dehydrogenase deficiency, Familial LPL deficiency, Protein S deficiency, Tyrosinemia type I, Adenine phosphoribosyltransferase deficiency, Citrullinemia type I, Methylmalonic semialdehyde dehydrogenase deficiency, Succinyl-CoA 3-oxoacid-CoA transferase deficiency, Galactose-1-phosphate uridyl transferase deficiency, Glycerol kinase deficiency, Nonketotic hyperglycinemia, Glutaric acidemia type I, Molybdenum cofactor defect, Zellweger syndrome, Cystinosis, T2D, Hemophilia A or B, Stickler syndrome, Osteoporosis, Rheumatoid Arthritis, A1AT deficiency, Breast cancer, Atherosclerosis, Isobutyryl-CoA dehydrogenase deficiency, carnitine-acylcarnitine translocase deficiency, Sitosterolemia, Phenylketonuria, Hereditary xanthinuria, Ornithine-transcarbamoylase deficiency, 3-Hydroxy-3-methylglutaryl-CoA synthase deficiency, Nonketotic hyperglycinemia, Hyperlysinemia, Homocystinuria, Refsum disease, and growth failure in children with kidney disease.

In some embodiments, the adipogenic cells are CD34⁺ cells and the disease or disorder is selected from a disease or disorder selected from Lysosomal storage disorders, Metabolic disorders, Hematological disorders, Bone and connective tissue disorders, Endocrine disorders, Inflammatory disorders, Monogenic disorders, Cancer, Cardiovascular disorders, Branched-chain amino acid metabolism disorders, Fatty acid transport and mitochrondrial oxidation disorders, Genetic dyslipidemias, Phenylalanine and tyrosine metabolism disorders, Purine metabolism disorders, Urea cycle disorders, Ketone metabolism disorders, Glycine Metabolism Disorders, Lysine Metabolism Disorders, Methionine and Sulfur Metabolism Disorders, Peroxisome biogenesis and very long chain fatty acid metabolism disorders, other protein deficiency disorders, Complement deficiencies, Adipocyte disorders, Vascular diseases, Connective tissue disorders, Beta-amino acid and gamma-amino acid disorders, Galactosemia, and Glycerol Metabolism Disorders.

In some embodiments, the adipogenic cells and/or compositions comprising same are administered in combination with one or more additional compounds. In some embodiments, the adipogenic cells are pretreated with one or more additional compounds, for example prior to administration to a subject. In some embodiments, the one or more compounds are additional therapeutic agents. In some embodiments the one or more additional compounds include small molecules, large molecules, and/or extracts. Non-limiting embodiments of small molecules include VEGF activators, such as TGP-377; HIF-1alpha activators/stabilizers, such as 3,4 DHB, L-mimosine, DBM, Ciclopirox olamine, DFO, NOG, and DMOG; LPA-agonists such as 2(S)-OMPT, adenosine receptor agonists, beta-lactams, such as penicillins and cephalosporin C; macrolides, such as erythromycin; aminoglycosides such as streptomycin; resveratrol; ginsenosides such as Rb1, Rb2, Rg3, Rh2, Rh3, Rg1, Rg2, Rh1, and F1; curcumin; adenosine; sokotrasterol sulfate; and cholestane trisulfate. Non-limiting examples of large molecules include VEGFA; VEGF165; FGF2; FGF4; PDGF-BB (platelet-derived growth factor); Ang1 (angiopoiten 1), TGFβ (transforming growth factor); LPA-producing enzyme (AXT); phthalimide neovascularization factor (PNF1). Non-limiting embodiments of extracts include extracts of Epimedium sagittatum, extracts of Trichosanthes kirilowii and extracts of Dalbergia odorifera.

In one aspect, the present invention includes a process for in vivo electroporation (EP) of adipogenic cells. Electroporation is a method for permeabilization of cell membranes by temporary generation of membrane pores with electrical stimulation. In some embodiments, the method comprises injecting the adipogenic cells into adipose tissue of a subject, placing the adipose tissue between a first plate electrode and a second plate electrode, and passing a current from the first plate electrode through the adipose tissue to the second plate electrode. In some embodiments, the tissue is folded between the first plate electrode and the second plate electrode.

In some embodiments, the current is a series of electrical pulses. In some embodiments, the plate electrodes each have a voltage between about 150 cm⁻¹ and about 350 cm⁻¹. In some embodiments, the plate electrodes each have a voltage between about 175 cm⁻¹ and about 300 cm⁻¹. In some embodiments, the plate electrodes each have a voltage between about 190 cm⁻¹ and about 250 cm⁻¹. In some embodiments, the plate electrodes each have a voltage between about 195 cm⁻¹ and about 210 cm-1. In some embodiments, the plate electrodes each have a voltage up to about 155 V, about 160 V, about 165 V, about 170 V, about 175 V, about 180 V, about 185 V. about 190 V, about 195 V, about 200 V, about 205 V, about 210 V, about 215 V, about 220 V, about 225 V, about 230 V, about 235 V, about 240 V, about 245 V, about 250 V, about 255 V, about 260 V, about 265 V, about 270 V, about 275 V, about 280 V, about 295 V, or about 300 V.

In some embodiments, the distance between the first plate electrode and the second plate electrode ranges from about 5 mm to about 50 mm, about 5 mm to about 20 mm, or about 10 mm to about 15 mm. In some embodiments, the distance between the first plate electrode and the second plate electrode is about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm. See, e.g., Fisher et al. Gene Therapy 24:757-767 (2017), which is incorporated by reference herein in its entirety.

Subjects and/or Animals

In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g. GFP). In some embodiments, the subject and/or animal is a transgenic animal comprising a fluorescent cell, such as, for example, an RPE cell and/or an immune cell. In some embodiments, the subject and/or animal is a human. In some embodiments, the human is a pediatric human. In some embodiments, the human is an infant or child. In some embodiments, the human is an adult human. In some embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal.

In various embodiments, a subject's and/or an animal's eye comprises (i) a fluorescent compound in an amount effective to indicate the presence of an ocular disease or disorder in the subject and/or animal and (ii) a toxin in an amount effective to induce atrophy of ocular tissue. In some embodiments, such a subject and/or animal is administered an agent of the invention or is not administered an agent of the invention.

In various embodiments, RPE and immune cells are evaluated and/or effected. In some embodiments, immune cells include cells of a subject's and/or animal's innate immune system. In some embodiments, such cells include, but are not limited to, macrophage, monocyte, and microglial cells. In various embodiments, the invention provides for detecting a presence, detecting an absence, or measuring an amount of immune cells in a subject's and/or animal's eye

### Kits

The invention provides kits that can simplify the administration of any agent described herein. An exemplary kit of the invention comprises any agent described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the ocular surface. The kit can also further comprise one or more additional agent described herein.

In one aspect, the present invention includes a syringe comprising one or more compositions of the present invention. In some embodiments, the syringe is prefilled with a volume of the composition. In some embodiments, the syringe is prefilled in a volume of about 1 mL to about 10 mL. In some embodiments, the syringe is prefilled in a volume of about 6.0 mL, about 5.9 mL, about 5.8 mL, about 5.7 mL, about 5.6 mL, about 5.5 mL, about 5.4 mL, about 5.3 mL, about 5.2 mL, about 5.1 mL, about 5.0 mL, about 4.9 mL, about 4.8 mL, about 4.7 mL, about 4.6 mL, about 4.5 mL, about 4.4 mL, about 4.3 mL, about 4.2 mL, about 4.1 mL, about 4.0 mL, about 3.9 mL, about 3.8 mL, about 3.7 mL, about 3.6 mL, about 3.5 mL, about 3.4 mL, about 3.3 mL, about 3.2 mL, about 3.1 mL, about 3.0 mL, about 2.9 mL, about 2.8 mL, about 2.7 mL, about 2.6 mL, about 2.5 mL, about 2.4 mL, about 2.3 mL, about 2.2 mL, about 2.1 mL, about 2 mL, about 1.9 mL, about 1.8 mL, about 1.7 mL, about 1.6 mL, about 1.5 mL, about 1.4 mL, about 1.3 mL, about 1.2 mL, about 1.1 mL, or about 1.0 mL or less of the composition. In some embodiments, the syringe is prefilled with a volume less than about 10 mL of the composition. In some embodiments, the syringe is prefilled with a volume less than about 6 mL of the composition. In some embodiments, the syringe is prefilled with a volume less than about 3 mL of the composition. In some embodiments, the syringe is prefilled with a volume of about 2 mL or less of the composition.

In some embodiments, the syringe comprises a composition having a shelf stability ranging from about 2 hours to about 1 week. In some embodiments, the syringe comprises a composition having a shelf stability of at least about 12 hours, about 24 hours, about 36 hours, about 48 hours, or about 72 hours when stored at a temperature ranging from about −85° C. to about 25° C. In some embodiments, the syringe comprises a composition having a shelf stability ranging from about 2 hours to about 1 week. In some embodiments, the syringe comprises a composition having a shelf stability of at least about 12 hours, about 24 hours, about 36 hours, about 48 hours, or about 72 hours when stored at a temperature ranging from about 15° C. to about 25° C.

In some embodiments, the syringe comprises a composition exhibiting less than about 35%, about 30%, about 25%, about 20%, about 19%, about 18%, about 17%, about 16%, about 15%, about 14%, about 13%, about 12%, about 11%, about 10%, about 9%, about 8%, about 7%, about 6%, or about 5% loss of cell viability when stored at a temperature ranging from about −85° C. to about 25° C. In some embodiments, In some embodiments, the syringe comprises a composition exhibiting less than about 35%, about 30%, about 25%, about 20%, about 19%, about 18%, about 17%, about 16%, about 15%, about 14%, about 13%, about 12%, about 11%, about 10%, about 9%, about 8%, about 7%, about 6%, or about 5% loss of cell viability when stored at a temperature ranging from about 15° C. to about 25° C.

In some embodiments, the storage temperature is about −80° C. In some embodiments, the storage temperature is about −20° C. In some embodiments, the storage temperature is about 4° C. In some embodiments, the storage temperature is about 21° C.

In one embodiment, the kit comprises a container containing a composition comprising adipogenic cells of the present invention, and a therapeutically effective amount of an additional therapeutic agent, such those described herein.

### Definitions

The following definitions are used in connection with the invention disclosed herein. Unless defined otherwise, all technical and scientific terms used herein have the same meaning as commonly understood to one of skill in the art to which this invention belongs.

An “effective amount” is an amount that is effective for treating, preventing, or ameliorating a disease or disorder such as those described herein.

An agent is “useful for the treatment of a disease or disorder” if the agent provides a measurable treatment, prevention, or reduction in the rate of pathogenesis of a disease or disorder.

As used herein, “a,” “an,” or “the” can mean one or more than one.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

In embodiments, adipose tissue includes any fat tissue. The adipose tissue may be brown or white adipose tissue, derived from subcutaneous, omental/visceral, mammary, gonadal, or other adipose tissue site. In some embodiments, the adipose tissue is subcutaneous white adipose tissue. The adipose tissue may be from any organism having fat tissue. In some embodiments, the adipose tissue is mammalian. In some embodiments, the adipose tissue is human. A convenient source of adipose tissue is from liposuction surgery, however, the source of adipose tissue or the method of isolation of adipose tissue is not limited.

In embodiments, adipogenic cells are cells that, upon administration to a subject, preferentially provide adipocytes. In some embodiments, adipogenic cells are adipocytes, whether white or brown/beige; in certain particular embodiments, the adipocytes are white adipocytes. In other embodiments, adipogenic cells are adipose-derived stem cells (ASCs). In still other embodiments, the adipogenic cells are CD34⁺ cells. Adipogenic cells can thus include precursor or progenitor cells to any of the foregoing, such as pre-adipocytes, pre-ASCs, and MSCs. Adipocytes, or commonly fat cells, can be characterized by a variety of properties. In some embodiments, adipocytes are characterized by expression (e.g., elevated expression) or one or more genes, including CIDEC, FABP4, PLIN1, LGALS12, ADIPOQ, TUSC5, SLC19A3, PPARG, LEP, CEBPA, and combinations thereof. In some embodiments, adipocytes are characterized as having one or more, 2 or more, 3 or more, 4 or more, 5 or more, 10 or more, 15 or more, 20 or more, 25 or more, 30 or more, or 35 or more of the following:
a. being post-mitotic;
b. having a lipid content of greater than about 35% (% fresh weight of adipose tissue; e.g. greater than about 40%, about 45%, about 50%, about 55%, about 60%, about 65%, about 70%, about 75%, or about 80%); optionally having a fat content in adipose tissue of about 60% to about 95% (e.g. 60-94%, about 60% to about 90%, about 60% to about 85%, about 60% to about 80%, about 60% to about 75%, about 60% to about 70%, about 60% to about 65%, about 65% to about 90%, about 70% to about 90%, about 75% to about 90%, about 80% to about 90%, or about 85% to about 90%), optionally having an average fat content of about 80% (e.g. about 75 to about 85%), optionally having a water content in adipose tissue of about 5% to about 40% (e.g. about 6-36%, about 5% to about 35%, about 5% to about 30%, about 5% to about 25%, about 5% to about 20%, about 5% to about 15%, about 5% to about 10%, about 10% to about 40%, about 15% to about 40%, about 20% to about 40%, about 25% to about 40%, about 30% to about 40%, or about 35% to about 40%), optionally having an average water content of about 15% (e.g. about 12.5% to about 17.5%), and optionally having a specific gravity of about 1 g/mL (e.g. 0.916 g/mL, about 0.5 g/mL, about 0.6 g/mL, about 0.7 g/mL, about 0.8 g/mL, about 0.9 g/mL, about 1.1 g/mL, or about 1.2 g/mL);
c. having a lipid content comprising one or more of free fatty acids, cholesterol, monoglycerides, and diglycerides;
d. having a lipid content comprising one or more of stearic acid, oleic acid, linoleic acid, palmitic acid, palmitoleic acid, and myristic acid, a derivative thereof;
e. having a lipid droplet of a size greater than about 90% of the cell volume (e.g. greater than 95% or greater than about 98%, or about 93%, or about 95%, or about 97%, or about 99%);
f. having a lipid droplet comprising at least about 30% to about 99% of the volume of the cell; (e.g., at least about 40% to about 90%, about 50% to about 90%, about 60% to about 90%, about 70% to about 90% about 80% to about 90%, about 50%, about 60%, about 70%, about 80%, or about 90%);
g. having a surface size of about 20-300 μm in diameter (e.g. about 20-300 μm, about 20-200 μm, about 20-100 μm, about 20-500 μm, about 20-30 μm, about 50-300 μm, about 50-200 μm, about 50-100 μm, about 100-300 μm, about 100-200 μm, about 150-300 μm, about 150-200 μm, or about 200-300 μm);
h. having a nucleus volume of about 200-400 μm³ (e.g. about 200 to about 350 μm³, about 200 to about 300 μm³, about 200 to about 250 μm³, about 250 to about 400 μm³, about 250 to about 350 μm³, about 250 to about 300 μm³, about 300 to about 350 μm³ or about 300 to about 400 μm³);
i. having a total volume of about 4,000-18,000 μm³ (e.g. about 4000 to about 15000 μm³, about 5000 to about 15000 μm³, about 10000 to about 15000 μm³, about 12500 to about 15000 μm³, about 4000 to about 10000 μm³, about 5000 to about 15000 μm³, about 7500 to about 15000 μm³, about 10000 to about 15000 μm³, about 12500 to about 15000 μm³);
j. having a nucleus to cell ratio of about 1:20-1:90 (e.g. about 1:20 to about 1:80, about 1:20 to about 1:70, about 1:20 to about 1:60, about 1:20 to about 1:50, about 1:20 to about 1:40, about 1:20 to about 1:30; about 1:30 to about 1:80, about 1:40 to about 1:80, about 1:50 to about 1:80, about 1:60 to about 1:80, or about 1:70 to about 1:80);
k. having a flattened nucleus;
l. having a small cytoplasm of less than about 10% to about 60% of total cell volume, wherein the cytoplasm excludes lipid droplets volume (e.g. less than about 20%, less than about 30%, less than about 40%, or less than about 50%);
m. being capable of absorbing and releasing liquids;
n. being buoyant in in water or an aqueous solution (e.g., media, or HBSS);
o. having a non-centrally located nucleus;
p. having one or more fat droplets;
q. having a non-spherical cytoplasm;
r. being capable of secreting one or more of adiponectin, leptin, and TNF-alpha;
s. being capable of lipogenesis;
t. being capable of storing triglycerides (TG);
u. being capable of secreting non-esterified fatty acids NEFA) (e.g., long chain fatty acids such as oleic acid palmitoleic acid, linoleic acid, arachidonic acid, lauric acid, and stearic acid);
v. being responsive to hormones;
w. being responsive to neural input;
x. having a cell turn-over rate of about 9 years (e.g. about 8 to about 10 years);
y. having an average diameter of about 45 μm (e.g. about 47.2 μm, about 40 μm, about; 42.5 μm, about 47.5 μm, or about 50 μm)
z. a cell population having a diameter distribution wherein about 25% of cells have a diameter of less than about 50 μm; about 40% of cells have a diameter of about 50-69 μm; about 25% of cells have a diameter of about 70-89 μm, and about 10% of cells have a diameter of greater than or equal to about 90 μm;
aa. responsive to atrial natriuretic peptide (ANP);
bb. capable of lipolysis;
cc. expressing receptors that can bind and respond to steroid hormones;
dd. lysed due to phosphatidylcholine;
ee. cell density of about 1 g/ml (e.g. about 0.8 g/ml, about 0.9 g/ml, about 1.1 g/ml, about 1.2 g/ml);
ff. greater than about 80% viability (e.g. about 85%, about 90%, about 95%, about 97%, about 98%, or about 99%);
gg. greater than about 80% purity (e.g. about 85%, about 90%, about 95%, about 97%, about 98%, or about 99%),
hh. adequate potency (e.g. amount of Oil Red O eluted greater than about 200 μg/ml); and
ii. negative for microbial contamination.

In embodiments, adipose stem cells, also referred to as adipose-derived stem cells or ASCs, are stem cells that originate from the stromal fraction of adipose tissue, generally from a mammal, such as human, i.e., human adipose tissue (hASCs). In some embodiments, the ASCs are positive for one or more of the surface markers CD29, CD73, CD90, and CD105 (e.g., positive for 1, 2, 3, or all 4); in certain embodiments, the ASCs negative for one or more of the surface markers CD31 and CD45 (e.g., negative for one or both); while in further embodiments, ASCs are positive for one or more of the surface markers CD29, CD73, CD90, and CD105 (e.g., positive for 1, 2, 3, or all 4) and negative for one or more of the surface markers CD31, CD34, and CD45 (e.g., negative for one, two, or all three). ASCs, in some embodiments are adherent to plastic under standard culture conditions. Expanded ASCs, in certain embodiments exhibit a fibroblast-like morphology in culture. ASCs are, in some embodiments, characterized by the ability to differentiate toward one or more of the osteogenic, adipogenic, myogenic, or chondrogenic lineages.

In some embodiments, the adipogenic cells are positive for one or more of the surface markers CD90, CD73 and MHC-I (e.g., positive for 1, 2, or all 3); in certain embodiments, the adipogenic cells are negative for one or more of the surface markers MHC-II, CD45 and CD40 (e.g., negative for 1, 2, or all 3); while in further embodiments, adipogenic cells are positive for one or more of the surface markers CD90, CD73 and MHC-I (e.g., positive for 1, 2, or all 3) and negative for one or more of the surface markers MHC-II, CD45 and CD40 (e.g., negative for 1, 2, or all 3). In some embodiments, the adipogenic cells are ASCs. In some embodiments, the adipogenic cells are adipocytes. Throughout this disclosure, the terms “MHC” and “HLA” may be used interchangeably.

In embodiments, CD34⁺ cells refer to cells positive for the surface marker CD34. In some embodiments, CD34 cells are also positive for one or more of CD90 and CD49F (e.g., one or both). In certain embodiments, CD34⁺ cells are negative for one or more of Lin, CD38, and CD45RA (e.g., negative for one, two or all three). In still other embodiments, CD34⁺ cells are positive for one or both of CD90 and CD49F and negative for one or more of Lin, CD38, and CD45RA. In certain embodiments, these cells are hematopoietic stem cells and progenitor cells, such as hematopoietic progenitor cells and endothelial progenitor cells. Human CD34⁺ cells are relatively rare cells, normally found in bone marrow in adults. These cells give rise to all major hematopoietic lineages. Besides CD34, they are typically positive for surface markers CD90 and CD49F and negative for Lin, CD38, and CD45RA.

This invention is further illustrated by the following non-limiting examples

### EXAMPLES

### Example 1: Isolation of ASCs and Cell Expansion in Culture

This example demonstrates, inter alia, the process of isolating ASCs from adipose tissues and expanding ASCs in culture.

In this example, the ASCs were isolated from adipose tissue using either an enzymatic digestion method or an explant culture method. The adipose tissue was subcutaneous white adipose tissue, isolated via the standard liposuction procedure from a human donor or surgically removed from mice. See Wu et al., Clevel. Clin. J. Med. 87, 6, 367-476 (2020) and Kilroy et al., Isolation of murine adipose-derived stromal/stem cells for adipogenic differentiation or flow cytometry-based analysis, Adipose-derived stem cells: Methods and protocols. 2nd ed. New York (NY): Springer Nature, 137-146 (2018), both of which are incorporated by reference herein in their entireties. The enzymatic digestion method was as follows. The adipose tissue was washed three or four times with PBS and suspended in an equal volume of 0.1% collagenase type I (Sigma-Aldrich, SCR103). Digestion was performed at 37° C. with 5% humidified CO₂ and continuous agitation for 60 min, following which enzyme was neutralized with FBS. The digest was then centrifuged for 20 min at 400×g. The supernatant was discarded, and the pellet was washed twice with complete medium (DMEM with low glucose, supplemented with 10% FBS and penicillin-streptomycin) and filtered through a 100 μm cell strainer (Falcon, 352360). The cells were plated in complete medium at a density of 1×10⁴-2×10⁴ cells/cm² and maintained at 37° C. with 5% humidified C02. Non-adherent cells were removed by replacing the culture medium after 24 hours, and the plastic adherent cells were expanded with change of culture medium every 3-4 days. Cells were expanded up to duplication 15 and frozen.

The explant culture method for isolating ASCs was as follows. The adipose tissue was washed to remove excess blood by mixing with an equal volume of PBS and allowed to settle for 5 min for separation of the aqueous phase from the fat fraction. The fat was then transferred to a Petri dish, where it was minced into fragments of about 5 mm³. The tissue fragments were evenly distributed over the surface of a tissue culture-treated dish. Approximately 1 g tissue was plated per 100 mm dish. 2.5 ml of prewarmed complete medium was gently added to the dish such that the explants still remain in contact with the surface of the culture dish. The dish was maintained at 37° C. with 5% humidified CO₂ with a change of medium every 3-4 days. Cell outgrowth was observed on day 5-10 after plating, and the explant tissue was removed after another 5-7 days. The outgrown cells were expanded up to duplication 15 and frozen.

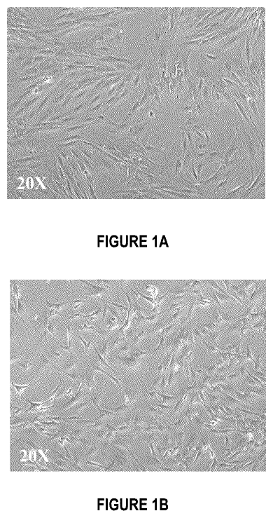

ASCs were successfully isolated and expanded in culture. FIG. 1A-B shows representative images of ASCs in culture (FIG. 1A: human ASCs; FIG. 1B: murine ASCs) taken using transmitted light and the 20× objective in an EVOS M5000 imaging system (ThermoFisher). The cells are adherent to the tissue culture dish surface and display typical ASC morphology of spindle shape and large, flattened appearance.

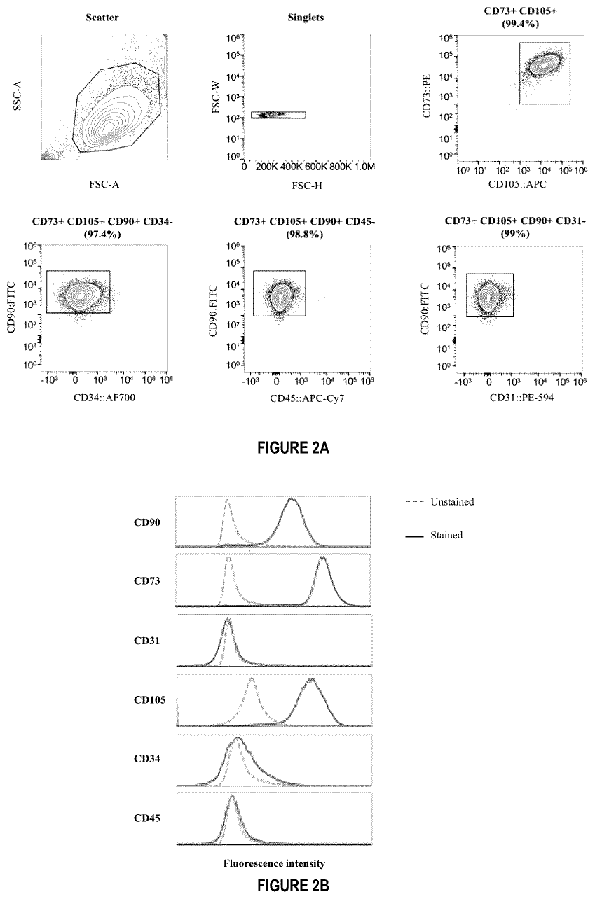

The isolated and expanded cells were characterized for ASCs' surface markers using flow cytometric analysis. Specifically, cells were stained with directly conjugated antibodies against CD29, CD73, CD90, CD105, CD31, CD45, and CD34. It was expected that the isolated cells would show high expression of CD29, CD73, CD90, and CD105, low expression of CD31 and CD45, and variable expression of CD34. FIG. 2A-B shows that the ASCs constitute a relatively homogenous population and >97% of the ASCs are positive for CD73, CD105, and CD90 and negative for CD34, CD45, and CD31.

Overall, this example demonstrates, inter alia, ASCs were successfully isolated from adipose tissues, could be expanded the cells in culture, and were characterized based on the expressions of cell surface markers.

### Example 2: Isolation of Human CD34⁺ Cells and Cell Expansion in Culture

This example demonstrates, inter alia, the process of isolating human CD34⁺ cells from peripheral blood and cell culture expansion.

In this example, CD34⁺ cells are isolated from a human donor as follows. CD34⁺ cells mobilization from the bone marrow is performed using filgrastim (granulocyte-colony stimulating factor; G-CSF) and plerixafor. Peripheral blood mononuclear cells are collected by apheresis. Harvested cells are enriched for CD34⁺ cells with the use of a CliniMACS device (Miltenyi Biotec) according to the manufacturer's instructions. The cells are cultured in Stem Cell Growth Medium (SCGM, Cell Genix) supplemented with the following cytokines: 100 ng/mL thrombopoietin (TPO), 100 ng/mL Fms-related tyrosine kinase 3 ligand (FltL), and 100 ng/mL stem cell factor (SCF) (all from Cell Genix). The cells are maintained at 37° C. with 5% humidified CO₂ and diluted with fresh medium everyday as required to maintain proper cell density ranging from 1×10⁵ to 5×10⁵ cells/mL. Cells are maintained in culture up to 1 week and frozen.

Freshly isolated cells and cultured cells are characterized for surface markers using flow cytometric analysis. Specifically, cells are stained with a directly conjugated antibody against CD34, CD90, CD49F, Lin, CD38, or CD45RA (Biolegend). It is expected that the cells will show high expression of CD34, CD90, and CD49F and low expression of Lin, CD38, and CD45RA.

Overall, this example shows, inter alia, that CD34⁺ cells can be isolated from human mobilized peripheral blood, expanded in culture, and characterized.

### Example 3: In Vitro Production of Adipocytes by Differentiation of ASCs

This example demonstrates, inter alia, the process of adipogenic differentiation to obtain adipocytes from ASCs.

ASCs were isolated and expanded in culture as described in Example 1. Adipocytes were derived from ASCs using a procedure modified from Li et al., Isolation of human adipose-derived stem cells from lipoaspirates, Adipose-derived stem cells: Methods and protocols. 2nd ed. New York (NY): Springer Nature. p. 155-165 (2018), which is incorporated by reference herein in its entirety. The expanded ASCs at 100% confluence were treated with the following differentiation medium: DMEM/F12 (Gibco, 10565042) supplemented with 10% FBS, 33 μM biotin (Fisher, BP232-1), 17 μM pantothenate (Fisher, AAA1660922), 1 μM bovine insulin (Sigma, 10516), 1 μM dexamethasone (Fisher, D19611G), 0.1875 mM isobutylmethylxanthine (IBMX) (Fisher, AC228420010), and 0.2 mM indomethacin (Fisher, AAA1991006). The human ASCs were kept on the differentiation medium for 7-8 days. On the other hand, after 3 days of adipogenic induction, the murine ASCs were fed the same medium without IBMX and indomethacin for an additional 4-5 days. The differentiated ASCs were harvested by incubation with 0.25% Trypsin-EDTA for 5-10 minutes at 37° C. Trypsin-EDTA is inactivated by the addition of DMEM (+10% FBS). For cryopreservation, the harvested cells were resuspended in cryopreservation medium (90% FBS, 10% DMSO) at 2.5×10⁷ cells/mL and immediately placed into a freezing container with the temperature at −80° C., overnight, and then transferred to a liquid nitrogen tank (−140° C.) for storage.

The adipogenic differentiation was assessed for the presence of intracellular lipid droplets by observing the cellular morphology through Oil Red O staining. Specifically, the cells were fixed in 10% (v/v) neutral buffered formaldehyde (Sigma, HT501128) for 1 h and stained for 10 min with a 60% (v/v) Oil Red O solution (Fisher, AAA1298914). The rate of differentiation was expressed as the ratio of the number of Oil Red O-positive cells to the number of total cells.

The levels of adipocyte-specific gene expressions in the differentiated cells were quantified by reverse transcription-polymerase chain reaction (RT-PCR). Total RNA was isolated from cells using a phenol-based extraction reagent (Invitrogen) and subjected to reverse transcription to generate cDNA. qRT-PCR analysis was performed using a dye-based quantitative PCR mix (BioRad). The following adipogenic genes were assayed using the listed primer pairs: adiponectin (human: primers 1 and 2; murine: primers 3 and 4), PPARγ (human: primers 5 and 6; murine: primers 7 and 8), leptin (human: primers 9 and 10; murine: primers 11 and 12), CIDEC (human: primers 13 and 14; murine: primers 15 and 16), FABP4 (human: primers 17 and 18; murine: primers 19 and 20). GAPDH (human: primers 21 and 22; murine: primers 23 and 24) and actin (human: primers 25 and 26; murine: primers 27 and 28) were used as controls.

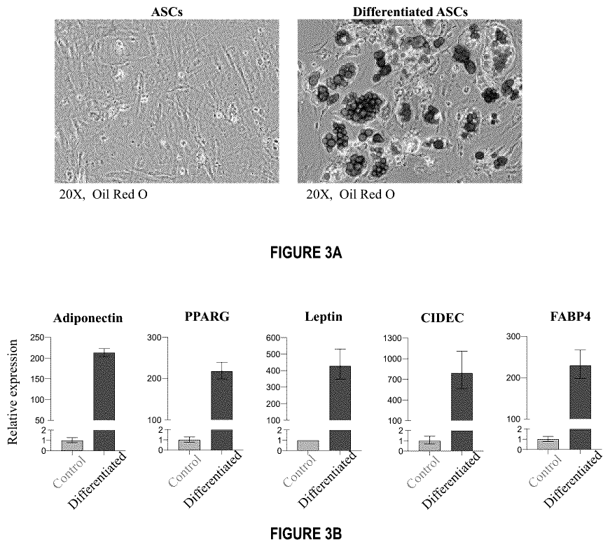

As shown in FIG. 3A, more than 80% of ASCs were differentiated into adipocytes, which contain lipid droplets stained positive for Oil Red O. In addition, FIG. 3B shows that all tested adipocyte-specific genes are highly upregulated in the differentiated cells, further confirming adipogenic differentiation.

The efficiency of adipogenic differentiation is also quantified via flow cytometric analysis. Specifically, LipidTOX Deep Red (Fisher, H34477) is added to the cell suspension at 1:200 dilution and mixed gently. The cells are incubated at room temperature for 30 min. The cells are then analyzed on a flow cytometer. It is expected that differentiated adipocytes are stained for LipidTOX at a higher level compared to ASCs. The LipidTOX-positive cells can also be quantified via cell imaging using an epifluorescence microscope.

Overall, this example details, inter alia, the steps to differentiate ASCs into adipocytes in culture. The example also demonstrates how to assess the adipogenic differentiation and characterize the differentiated cells.

### Example 4: In Vitro Production of Adipocytes by Differentiation of CD34⁺ Cells

This example demonstrates, inter alia, the process of adipogenic differentiation to obtain adipocytes from CD34⁺ cells.

CD34⁺ cells are isolated and expanded in culture as described in Example 2. Adipocytes are derived from CD34⁺ cells as follows. The CD34⁺ cells are cultured in minimum essential medium α (αMEM) (Gibco, 12571063) containing 20% FBS, 15 ng/mL interleukin-3 (IL-3) (Gibco, PHC0034), and 0.6 ng/mL recombinant human macrophage-colony stimulating factor (human M-CSF) (R&D Systems, 216-MC) for a period of 3 days. The non-adherent cells are treated with 0.02% Pronase (MilliporeSigma) and then cultured in αMEM containing 20% FBS and 10 ng/mL of M-CSF for a period of 2 days. To differentiate the adherent cells into adipocytes, complete growth medium is replaced with adipogenesis initiation medium consisting of αMEM, 10% FBS, 100 ng/mL human M-CSF, 0.5 mM IBMX (Fisher, AC228420010), and 1 μM dexamethasone (Fisher, D19611G), and 10 μg/mL of insulin (Sigma, 10516). After 2 days of induction, the medium is replaced with the adipogenesis progression medium consisting of αMEM, 10% FBS, 100 ng/mL human M-CSF, and 10 μg/mL insulin. 2 days later, the adipogenesis progression medium is replaced with the maintenance medium consisting of αMEM, 10% FBS, and 100 ng/mL human M-CSF, and incubation continues for at least 5 more days.

The adipogenic differentiation of CD34⁺ cells is assessed for the presence of intracellular lipid droplets by observing the cellular morphology through Oil Red O staining as described in Example 3. The expected adipogenic differentiation rate is 50-80%.

The efficiency of adipogenic differentiation for CD34⁺ cells can also be quantified via flow cytometric analysis as described in Example 3. It is expected that differentiated adipocytes are stained for LipidTOX at a higher level compared to ASCs. The LipidTOX-positive cells can also be quantified via cell imaging using an epifluorescence microscope.

The levels of adipocyte-specific gene expressions in the differentiated cells are quantified by reverse transcription-polymerase chain reaction (RT-PCR) as described in Example 3. It is expected that adipocytes will show higher expression levels of the adipogenic genes compared to ASCs.

Overall, this example details, inter alia, the steps to differentiate CD34⁺ cells into adipocytes in culture. The example also demonstrates, inter alia, how to assess the adipogenic differentiation and characterize the differentiated cells.

### Example 5A: Long-Term Engraftment of Adipocytes Derived from Transplanted ASCs in Mice and In Vivo Adiponectin Secretion

This example demonstrates, inter alia, the ability of transplanted ASCs to give rise to long-lasting adipocyte engraftment and secretion of adiponectin in vivo.

In this example, ASCs are isolated and expanded in culture as described in Example 1. Cryopreserved ASCs are thawed and seeded at 1×10⁵-3×10⁵ cells/cm² to allow cells to recover in culture from cryopreservation and not to expand. At 6-7 days, the cells are harvested and suspended in phenol red-free DMEM or Matrigel (Corning, 354234) at a concentration of 4×10⁶ cells/100 μL. Mice are anaesthetized using isoflurane prior to the cell injections. The dorsal side of each mouse is swabbed with 70% ethanol, and the ASCs suspended in phenol red-free DMEM or Matrigel (4×10⁶ cells/side) are injected using a 29 G gauge syringe into each side of the dorsal flank. In the mock-transplanted cohort, an equal volume of phenol red-free DMEM or Matrigel alone is injected. Post recovery, the mice are fed a normal chow diet (LabDiet, 5058) or a high fat diet (Research Diets, D12451).

In one cohort, eight-week old NOD SCID mice (homozygous for the severe combined immune deficiency spontaneous mutation Prkdc^{scid}, The Jackson Laboratory, 001303) or BALB/cJ mice (The Jackson Laboratory, 000651) are injected with ASCs derived from human adipose tissue (hASCs). Differentiation of hASCs into adipocytes in vivo is monitored via the serum level of human adiponectin since adiponectin is specific to adipocytes and is secreted into circulation. In these mice, serum is drawn every seven days for up to six months post recovery. Collected serum is diluted 1-10 fold in PBS and analyzed for human adiponectin by enzyme-linked immunosorbent assay (Zen-Bio, Inc., ADIP-1). It is expected that the serum level of human adiponectin in the transplanted mice will rise above the level in the mock-transplanted mice as early as the second week post recovery and will remain high up to six months.

In the same cohort, differentiation of hASCs into adipocytes in vivo is also assessed by the presence of human adipocytes in harvested tissues. Specifically, the hASCs-transplanted dorsal tissues, mouse adipose depots (gonadal, perirenal, retroperitoneal, mesenteric, and inguinal), and non-adipose depots (lower hind limb skeletal muscle, liver, and lung) are harvested seven days post recovery and every month afterward up to six months. The harvested tissues are subjected to whole-mount imaging on the same day of the cull. Specifically, the tissues are minced into ˜4 mm³ pieces and fixed in 1% paraformaldehyde for 15 min at room temperature. The fixed tissues are rehydrated in PBS 3×10 min each and stained with BODIPY-493/503 (ThermoFisher, D3922) (2 μg/ml to visualize the mature adipocytes), DAPI (ThermoFisher, D1306) (1 μg/ml, to visualize the nuclei), and anti-human CD29 antibody (1:25, to locate the human cells) (Biolegend) for 30 min on ice in the dark. The stained tissues are washed 3×10 min with PBS to remove any unbound dyes and antibody. The tissues are then placed on microscope slides and mounted with Fluoromount-G™ (ThermoFisher, 00-4958-02). The slides are imaged in an EVOS M5000 imaging system (ThermoFisher) using the 20× objective. The acquired images are processed in Adobe Photoshop software. Human adipocytes are cells stained positive for both BODIPY and human CD29. It is expected that these cells will appear in the hASCs-transplanted dorsal tissues as early as seven days post recovery. By 12 weeks, it is expected that fat pads are apparent at the transplanted sites. Human adipocytes may also be observed in mouse adipose and non-adipose depots due to the migration of the hASCs outside of the transplanted sites.

In a different cohort, eight-week old C57BL/6J mice (The Jackson Laboratory, 000664) are injected with ASCs derived from adipose tissue from UBC-GFP transgenic mice (The Jackson Laboratory, 004353) (GFP⁺ mASCs). Differentiation of GFP⁺ mASCs into GFP⁺ adipocytes is assessed by harvesting the grafted tissues, the recipient mouse adipose depots (gonadal, perirenal, retroperitoneal, mesenteric, and inguinal), and non-adipose depot (lower hind limb skeletal muscle, liver, and lung) seven days post recovery and every month afterward up to six months. As described above, the harvested tissues are minced into ˜4 mm³ pieces and fixed in 1% paraformaldehyde for 15 min at room temperature. The fixed tissues are rehydrated in PBS 3×10 min each and stained with BODIPY-493/503 (ThermoFisher, D3922) (2 μg/ml to visualize the mature adipocytes), DAPI (ThermoFisher, D1306) (1 μg/ml, to visualize the nuclei), and anti-GFP antibody (to locate the transplanted cells) (Biolegend). The stained tissues are then washed and imaged as described above. Adipocytes derived from the GFP⁺ mASCs are cells that stain positive for both GFP and BODIPY-493/503. Similar to the hASCs-transplanted cohort, it is expected that GFP⁺ mASCs-derived adipocytes will appear in the transplanted dorsal tissues as early as seven days post recovery. By 12 weeks, it is expected that fat pads are apparent at the transplanted sites. GFP⁺ mASCs-derived adipocytes may also be observed in the recipient mouse adipose and non-adipose depots due to the migration of the GFP⁺ mASCs outside of the transplanted sites.

Overall, the example demonstrates, inter alia, that both human and murine ASCs yield adipocytes upon transplantation, and the donor-derived adipocytes persist for up to six months in recipient mice. This example also shows, inter alia, the ability to achieve long-term in vivo secretion of human adiponectin by the human adipocytes derived from transplanted hASCs.

### Example 5B: Long-Term Engraftment of Adipocytes Derived from Transplanted ASCs in Mice and In Vivo

This example demonstrates, inter alia, the ability of transplanted human ASCs to give rise to long-lasting adipocyte engraftment in vivo as demonstrated by the detection of adipogenic genes Adipsin and FABP4 at day 117 post transplantation.

Prior to thawing cells, growth media was prepared with DMEM Low Glucose+Glutamax (Thermo Fisher, 10567-014) supplemented with 10% FBS (Gemini, 100-106) and 1× Penicillin-Streptomycin (Thermo Fisher, 15140-122) then sterile filtered through a 0.22 um filter bottle. A desired number of frozen ASC cryo-vials were collected from liquid nitrogen storage and thawed on a bead bath at 37 degrees. Once vials of ASCs were thawed, cell solutions were mixed with growth media at a ratio of 1 mL thawed cells to 9 mL of growth media then pelleted in a swinging bucket centrifuge at 200×g for 5 minutes. After centrifugation, media was carefully aspirated off without dislodging the pellet. Then the pellet was resuspended in 5 mL of growth media and gently mixed by pipetting up and down to the dislodge the pellet into single cells. After fully breaking the pellet into single cells, the cell solution was transferred to an appropriately sized sterile container and filled with a pre-determined volume of growth media for the size vessels to be used for culture. Cells were then seeded into at 3×10⁴-6×10⁴ cells/cm² to allow cells to recover in culture from cryopreservation and to expand. Growth media was changed the day after thawing cells followed by changes every 2-3 days until cells reach 70% confluence. Once cells reached 70% confluence, they were passaged as described above and seeded into 6 well culture plates at 1×10⁵ cells/well and allowed to culture overnight. The following day, cells were transfected with a pre-determined MOI, with a lentivirus reporter vector expressing a gLUC reporter gene with a puromycin resistance gene (engineered cells). gLUC expression was driven by the human adiponectin promoter (phAdipoQ) in hASCs. Engineered cells were selected using puromycin. Both engineered and unengineered cells were then further expanded.

Once cells reach 70% confluence they were harvested for transplantation. Growth media was aspirated off of the culture vessels and a desired volume of 0.25% Trypsin-EDTA (Thermo Fisher, 25200-072) was added on to each vessel. Vessels were then incubated at 37 degrees for 5 minutes to allow cells to dissociate off of the plastic. After 5 minutes cells were observed under a microscope at 4× to ensure there has been enough separation from the plastic. Cells were then fully dissociated from the plastic using a serological pipette to gently pipette the cell and trypsin solution up and down and washing across the span of the culture vessel. The cell solution was then transferred to an appropriately sized vessel leaving enough room for an equal volume of growth media. Culture vessels were then washed 1× using a serological pipette with an equal volume of growth media to ensure full removal of any residual cells on the culture vessels. Growth media was then transferred to the cell and trypsin solution to quench the trypsin. Cells were then pelleted by centrifuging in a swinging bucket centrifuge at 80×g for 5 minutes. After pelleting, media was removed and cells were resuspended in pre-chilled phenol-red free HBSS (Thermo Fisher, 14175-095) pipetted up and down using a serological pipette to break the pellet into single cells. After mixing thoroughly, 10 uL of cell solution were combined in a micro centrifuge tube with 10 uL of 0.4% Trypan Blue (Thermo Fisher, 15250-061) then counted using a Hemacytometer (Hausser Scientific, 3110) to determine a total viable cell count. After determining the total cell count, the cells were pelleted by centrifuging in a swinging bucket centrifuge at 80×g for 5 minutes. After pelleting, supernatant was aspirated off and cells were resuspended in pre-chilled HBSS to a final concentration of 4×10⁶ cells/100 uL.

NOD SCID mice (homozygous for the severe combined immune deficiency spontaneous mutation Prkdc^{scid}, The Jackson Laboratory, 001303) were injected with ASCs. The dorsal side of each mouse was swabbed with 70% ethanol, and the ASCs suspended in HBSS (4×10⁶ cells/side) were injected using a 25G gauge syringe into each side of the dorsal flank. In the mock-transplanted cohort, an equal volume of HBSS alone was injected. Post recovery, the mice were fed a high fat diet (Research Diets, D1245145% high fat diet product #NC9248609) for 14 days followed by normal chow diet (LabDiet, 5001) for the remainder of the study. Differentiation of hASCs into adipocytes in vivo was monitored via RT-PCR of human FABP4 and Adipsin at day 117 post-transplant in the dorsal flank. The levels of human adipocyte-specific gene expressions in the differentiated cells were quantified by reverse transcription-polymerase chain reaction (RT-PCR). Total RNA was isolated from cells using a phenol-based extraction reagent (Invitrogen) and subjected to reverse transcription to generate cDNA. qRT-PCR analysis was performed using a dye-based quantitative PCR mix (Applied Biosystems). The following adipogenic genes were assayed using the listed primer pairs: FABP4 (human: primers 17 and 18) and adipsin (human primers: Human Adipsin primers 108: GACACCATCGACCACGACC (SEQ ID NO: 34) and 109: GCCACGTCGCAGAGAGTTC (SEQ ID NO: 35)). Raw CT values were plotted, non-detected values were plotted at 40CT. As shown in FIGS. 6A-6B human FABP4 and Adipsin were detected at day 117 post-transplant in the dorsal flank. These markers are human specific and can thus not be derived from murine tissue. Both engineered and unengineered hASCs differentiated into adipocytes in vivo.

Overall, the example demonstrates, inter alia, that human ASCs yield adipocytes upon transplantation, and that the donor-derived adipocytes persist for more than 117 days in recipient mice.

### Example 5C: In Vivo Secretion of Gaussia Luciferase by Adipocytes Derived from Transplanted Genetically Modified Adipogenic Cells and Long-Term Engraftment of Adipocytes Derived from Transplanted Human ASCs in Mice (In Vivo)

This example demonstrates, inter alia, the ability to achieve sustained in vivo secretion of gaussia luciferase (GLuc) by transplanting engineered adipogenic cells. Furthermore, it demonstrates that transplanted engineered human ASCs give rise to long-lasting adipocyte engraftment in vivo as demonstrated by the detection of expression of gaussia luciferase under the adipocyte specific adiponectin promoter.

In this example, human ASCs hASCs were cultured similar to hASCs as described in Example 5A and/or 5B. Once cells reached 70% confluence, they were passaged as described in Example 5A and/or 5B and seeded into 6 well culture plates at 1×10⁵ cells/well and allowed to culture overnight. The following day, cells were transfected with a pre-determined MOI, with a lentivirus reporter vectors expressing a GLuc reporter gene with a puromycin resistance gene. GLuc expression was driven by the human adiponectin promoter SEQ ID NO: 4. hASCs were transfected using a pre-determined MOI by combining growth media with a calculated amount of the specific LV used. After 24 hours of transfection, growth media and LV was removed and replaced with fresh growth media. After 72 hours, LV1 Gluc cells were changed to new growth media containing 2 ug/mL Puromycin (Sigma, P9620) and allowed to culture for 96 hours to select for LV1 transfected cells. After 96 hours, substantial cell death was observed and all remaining cells were positively integrated with the LV1 construct. Cells were changed to new growth media and allowed to outgrow for 6-7 days until 70% confluence with media changes performed every 2-3 days. After reaching 70% confluence, transfected hASCs were passaged as described in Example 5A and/or 5B and allowed to outgrow for 6-7 days with media changes every 2-3 days. Cells were then passaged again as described in Example 5A and/or 5B and allowed to outgrow for 6-7 days with media changed every 2-3 days. After reaching 70% confluence, cells were passaged for differentiation as described in Example 7A and/or 7B and subsequently differentiated as described in Example 7A and/or 7B.

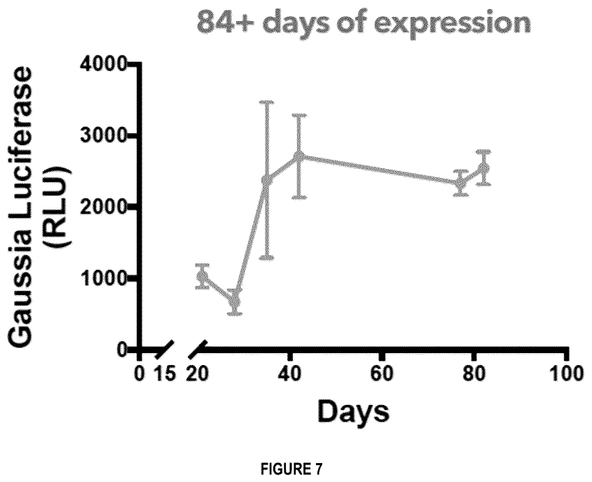

NOD SCID mice (The Jackson Laboratory, 001303) were injected with ASCs. The dorsal side of each mouse was swabbed with 70% ethanol, and the ASCs suspended in HBSS (8×10⁶ cells/side) were injected using a 25G gauge syringe into each side of the dorsal flank. In the mock-transplanted cohort, an equal volume of HBSS alone was injected. Post recovery, the mice were fed a high fat diet (Research Diets, D1245145% high fat diet product #NC9248609) for 28 days followed by normal chow diet (LabDiet, 5001) for the remainder of the study. Expression of adipocyte specific gluc was measured weekly in plasma. GLuc secretion was quantified using the Pierce™ Gaussia Luciferase Glow Assay kit (ThermoFisher, 16161) according to manufacturer's instructions. Briefly, the plasma was collected via a tail nick and mixed with a buffer containing coelenterazine. The bioluminescence produced by GLuc results from the oxidation of coelenterazine, and the signal was measured using a luminometer. As shown in FIGS. 7, donor-derived adipocytes express gluc for at least 84 days in recipient mice.

Overall, the example demonstrates, inter alia, that human ASCs yield adipocytes upon transplantation, and the donor-derived adipocytes persist for at least 84 days in recipient mice. This example also shows, inter alia, the ability to achieve long-term in vivo of gaussia luciferase by the adipocytes derived from transplanted hASCs.

### Example 6: Long-Term Engraftment of Adipocytes Derived from Transplanted CD34⁺ Cells and In Vivo Adiponectin Secretion

This example demonstrates, inter alia, the ability of transplanted CD34⁺ cells to give rise to long-lasting adipocyte engraftment and secretion of adiponectin in vivo.

In this example, human CD34⁺ cells are isolated and expanded in culture as described in Example 2. Cryopreserved CD34⁺ are thawed and pre-stimulated for 24-48 hours at approximately 1×10⁶ cells/mL in cytokine supplemented media (as described in Example 2). NOD.Cg-Kit^{W-41J} Tyr⁺ Prkdc^{scid} Il2rg^{tm1Wjl} (NBSGW) mice are obtained from the Jackson Laboratory (Stock 026622). Non-irradiated NBSGW female mice (6-8 weeks of age) are infused by retro-orbital injection with 0.2-0.8×10⁶ CD34⁺ cells (resuspended in 200 μl DPBS). Differentiation of transplanted human CD34⁺ cells into adipocytes in vivo is monitored via the serum level of human adiponectin since adiponectin is specific to adipocytes and is secreted into circulation. In these mice, serum is drawn every seven days for up to six months post recovery. Collected serum is diluted 1-10 fold in PBS and analyzed for human adiponectin by enzyme-linked immunosorbent assay (Zen-Bio, Inc., ADIP-1). It is expected that the serum level of human adiponectin in the transplanted mice will rise above the level in the mock-transplanted mice as early as the second week post recovery and will remain high up to six months.

Differentiation of human CD34⁺ cells into adipocytes in vivo is also assessed by the presence of human adipocytes in harvested tissues. Specifically, mouse adipose depots (gonadal, perirenal, retroperitoneal, mesenteric, and inguinal) and non-adipose depots (lower hind limb skeletal muscle, liver, and lung) are harvested seven days post recovery and every month afterward up to six months. The harvested tissues are subjected to whole-mount imaging on the same day of the cull. Specifically, the tissues are minced into ˜4 mm³ pieces and fixed in 1% paraformaldehyde for 15 min at room temperature. The fixed tissues are rehydrated in PBS 3×10 min each and stained with BODIPY-493/503 (ThermoFisher, D3922) (2 μg/ml to visualize the mature adipocytes), DAPI (ThermoFisher, D1306) (1 μg/ml, to visualize the nuclei), and anti-human CD29 antibody (1:25, to locate the human cells) (Biolegend) for 30 min on ice in the dark. The stained tissues are washed 3×10 min with PBS to remove any unbound dyes and antibody. The tissues are then placed on microscope slides and mounted with Fluoromount-G™ (ThermoFisher, 00-4958-02). The slides are imaged in an EVOS M5000 imaging system (ThermoFisher) using the 20× objective. The acquired images are processed in Adobe Photoshop software. Human adipocytes are cells stained positive for both BODIPY and human CD29. It is expected that these cells will appear in the mouse adipose depots as early as two weeks post recovery. Human adipocytes may also be observed in mouse non-adipose depots.

In additions, human CD34⁺ cells engraftment is assessed by harvesting bone marrow from the recipient mice 12-16 weeks post-engraftment. The bone marrow cells are analyzed using flow cytometry for the presence of human CD34⁺-derived cells. Specifically, the bone marrow cells are first incubated with Human TruStain FcX (422302, BioLegend) and TruStain fcX (anti-mouse CD16/32, 101320, BioLegend) blocking antibodies for 10 min, followed by the incubation with V450 Mouse Anti-Human CD45 Clone HI30 (560367, BD Biosciences), PE-eFluor 610 mCD45 Monoclonal Antibody (30-F11) (61-0451-82, Thermo Fisher), FITC anti-human CD235a Antibody (349104, BioLegend), PE anti-human CD33 Antibody (366608, BioLegend), APC anti-human CD19 Antibody (302212, BioLegend), and Fixable Viability Dye eFluor 780 for live/dead staining (65-0865-14, Thermo Fisher). Percentage human engraftment is calculated as hCD45⁺ cells/(hCD45⁺ cells+mCD45⁺ cells)×100. This number is expected to vary between 20% and 90%.

Overall, the example demonstrates, inter alia, that human CD34⁺ cells yield adipocytes upon transplantation, and the donor-derived adipocytes persist for up to six months in recipient mice. This example also shows, inter alia, the ability to achieve long-term in vivo secretion of human adiponectin by the human adipocytes derived from the transplanted CD34⁺ cells.

### Example 7A: Transplantation of Adipocytes and In Vivo Secretion of Adiponectin

This example demonstrates, inter alia, the process of transplanting adipocytes that lead to long-lasting cell engraftment and secretion of adiponectin in vivo.

In this example, adipocytes are derived from either ASCs as described in Example 3 or CD34⁺ cells as described in Example 4. Adipocytes are either freshly harvested or thawed from a cryopreserved stock. The cells are suspended at 10⁶ cells/40 μL in phenol red-free DMEM. Mice are anaesthetized using isoflurane prior to the cell injections. The dorsal side of each mouse is swabbed with 70% ethanol, and the adipocytes suspended in phenol red-free DMEM (4×10⁶ cells/side) are injected using a 26G gauge syringe into each side of the dorsal flank. In the mock-transplanted cohort, an equal volume of phenol red-free DMEM is injected.

In one cohort, eight-week old NOD SCID mice (The Jackson Laboratory, 001303) or BALB/cJ mice (The Jackson Laboratory, 000651) are injected with adipocytes derived from hASCs or human CD34⁺ cells in culture (hAdipocytes). Evidence for hAdipocyte engraftment is elevated serum human adiponectin levels and positive staining for both BODIPY-493/503 and human CD29 in grafted tissues following procedures described in Example 5A and/or 5B. Serum human adiponectin level is measured three days post recovery and then every week up to six months. Tissues are harvested and stained seven days post recovery and then every month up to six months. It is expected that serum human adiponectin levels will rise above baseline as early as three days post engraftment and will remain high up to six months. In contrast, no serum human adiponectin will be detected in the mock-transplanted mice. In addition, in the transplanted mice, cells stained positive for both BODIPY-493/503 and human CD29 are expected to persist in the grafted sites up to six months post engraftment.

In a different cohort, eight-week old C57BL/6J mice (The Jackson Laboratory, 000664) are injected with adipocytes derived from GFP⁺ mASCs in culture (GFP⁺ mAdipocytes). Evidence for GFP⁺ mAdipocyte engraftment is positive staining for both BODIPY-493/503 and GFP in grafted tissues following procedures described in Example 5A and/or 5B. Tissues are harvested and stained seven days post recovery and then every month up to six months. Cells stained positive for both BODIPY-493/503 and GFP are expected to persist in the grafted sites up to six months post engraftment.

In summary, the results from this example shows, inter alia, that adipocytes derived from human ASCs, murine ASCs, or human CD34⁺ cells in culture can be transplanted to achieve long-lasting adipocyte engraftment in vivo. This example also demonstrates, inter alia, the ability to achieve long-term in vivo secretion of human adiponectin from transplanted human adipocytes.

### Example 7B: Transplantation of Adipocytes and In Vivo Secretion of Adipsin

This example demonstrates, inter alia, the process of transplanting adipocytes in the subcutaneous layer in the skin and in the inguinal fat pad that leads to long-lasting cell engraftment and dose dependent secretion of adipsin in vivo.

ASCs were initially purchased from Obatala. Prior to thawing cells, growth media was prepared with DMEM Low Glucose+Glutamx (Thermo Fisher, 10567-014) supplemented with 10% FBS (Gemini, 100-106) and 1× Penicillin-Streptomycin (Thermo Fisher, 15140-122) then sterile filtered through a 0.22 um filter bottle. A desired number of frozen ASC cryo-vials were collected from liquid nitrogen storage and thawed on a bead bath at 37 degrees. Once vials of ASCs were thawed, cell solutions were mixed with growth media at a ratio of 1 mL thawed cells to 9 mL of growth media then pelleted in a swinging bucket centrifuge at 200×g for 5 minutes. After centrifugation, media was carefully aspirated off without dislodging the pellet. Then the pellet was resuspended in 5 mL of growth media and gently mixed by pipetting up and down to the dislodge the pellet into single cells. After fully breaking the pellet into single cells, the cell solution was transferred to an appropriately sized sterile container and filled with a pre-determined volume of growth media for the size vessels to be used for culture. Cells were then seeded into at 3×10⁴-6×10⁴ cells/cm² to allow cells to recover in culture from cryopreservation and to expand. Growth media was changed the day after thawing cells followed by changes every 2-3 days until cells reach 70% confluence. Once cells reach 70% confluence they were passaged to seed for differentiation. Growth media was aspirated off the culture vessels and a desired volume of 0.25% Trypsin-EDTA (Thermo Fisher, 25200-072) was added on to each vessel. Vessels were then incubated at 37 degrees for 5 minutes to allow cells to dissociate off of the plastic. After 5 minutes cells were observed under a microscope at 4× to ensure there has been enough separation from the plastic. Cells were then fully dissociated from the plastic using a serological pipette to gently pipette the cell and trypsin solution up and down and washing across the span of the culture vessel. The cell solution was then transferred to an appropriately sized vessel leaving enough room for an equal volume of growth media. Culture vessels were then washed 1× using a serological pipette with an equal volume of growth media to ensure full removal of any residual cells on the culture vessels. Growth media was then transferred to the cell and trypsin solution to quench the trypsin. Cells were then pelleted by centrifuging in a swinging bucket centrifuge at 80×g for 5 minutes. After pelleting, supernatant was removed, and cells were resuspended in a pre-determined volume of growth media. 10 μL of cells were then collected and mixed with 10 uL of 0.4% Trypan Blue (Thermo Fisher, 15250-061) and counted using a Hemocytometer (Hausser Scientific, 3110). After determining the total viable count, cells were then reseeded in a desired number of culture vessels at 41,666 cells/cm² and were cultured for 3 days. After 3 days of culture, differentiation of ASCs to Adipocytes begins. Sufficient Human Adipocyte Induction Media was prepared in DMEM/F12 (Thermo Fisher, 10565-018) containing 3% FBS (Gemini, 100-106), 1× Penicillin-Streptomycin (Thermo Fisher, 15140-122), 33 μM Biotin (Fisher Scientific, BP232-1), 17 μM Pantothenate (Fisher Scientific, AAA1660922), 1 μM Insulin (sigma, 19278), 187.5 uM IBMX (Fisher Scientific, AAJ64598MC), 200 uM Indomethacin (Fisher Scientific, AAA1991006), and 1 μM Dexamethasone (Fisher Scientific, D16911G) then sterile filtered through a 0.22 uM PES filter bottle. Growth media was then aspirated off culture vessels and replaced with freshly prepared Human Adipocyte Induction Media and then cultured for 3 days. After 3 days, sufficient Human Adipocyte Maintenance Media was prepared in DMEM/F12 (Thermo Fisher, 10565-018) containing 3% FBS (Gemini, 100-106), 1× Penicillin-Streptomycin (Thermo Fisher, 15140-122), 33 μM Biotin (Fisher Scientific, BP232-1), 17 uM Pantothenate (Fisher Scientific, AAA1660922), 1 μM Insulin (sigma, 19278), (Fisher Scientific, AAA1991006), and 1 μM Dexamethasone (Fisher Scientific, D16911G) then sterile filtered through a 0.22 μM PES filter bottle. Human Adipocyte Induction Media was aspirated off of the culture vessels and replaced with freshly prepared Human Adipocyte Maintenance Media and cultured for 4 days. After 7 days of differentiation, Human Adipocyte Maintenance Media was aspirated off of the culture vessels and a desired volume of 0.25% Trypsin-EDTA (Thermo Fisher, 25200-072) was added on to each vessel. Vessels were then incubated at 37 degrees for 5 minutes to allow cells to dissociate off the plastic. After 5 minutes cells were observed under a microscope at 4× to ensure there has been enough separation from the plastic. Cells were then fully dissociated from the plastic using a serological pipette to gently pipette the cell and trypsin solution up and down and washing across the span of the culture vessel. The cell solution was then transferred to an appropriately sized vessel leaving enough room for an equal volume of DMEM/F12 media. Culture vessels were then washed 1× using a serological pipette with an equal volume of DMEM/F12 to ensure full removal of any residual cells on the culture vessels. DMEM/F12 was then transferred to the cell and trypsin solution to quench the trypsin. Cells were then pelleted by centrifuging in a swinging bucket centrifuge at 80×g for 5 minutes. After pelleting, media was removed and cells were resuspended in pre-chilled phenol-red free HBSS (Thermo Fisher, 14175-095) pipetted up and down using a serological pipette to break the pellet into single cells. After mixing thoroughly, 10 uL of cell solution were combined in a micro centrifuge tube with 10 uL of 0.4% Trypan Blue (Thermo Fisher, 15250-061) then counted using a Hemacytometer (Hausser Scientific, 3110) to determine a total viable cell count. After determining the total cell count, the cells were pelleted by centrifuging in a swinging bucket centrifuge at 80×g for 5 minutes. After pelleting, supernatant was aspirated off and cells were resuspended in pre-chilled HBSS to a final concentration of 16 and 32×10⁶ cells/100 μL.

NOD SCID mice (The Jackson Laboratory, 001303) were injected with adipocytes derived from hASCs (hAdipocytes). The dorsal side of each mouse was swabbed with 70% ethanol, and the adipocytes suspended in HBSS (8, 16 or 32×10⁶ cells/side) were injected using a 27G gauge syringe into the side of the dorsal flank for subcutaneous dosing, or into the ingual fat pad. In the mock-transplanted cohort, an equal volume of HBSS alone was injected. Post recovery, the mice were fed a high fat diet (Research Diets, D1245145% high fat diet product #NC9248609) for 14 days followed by normal chow diet (LabDiet, 5001) for the remainder of the study.

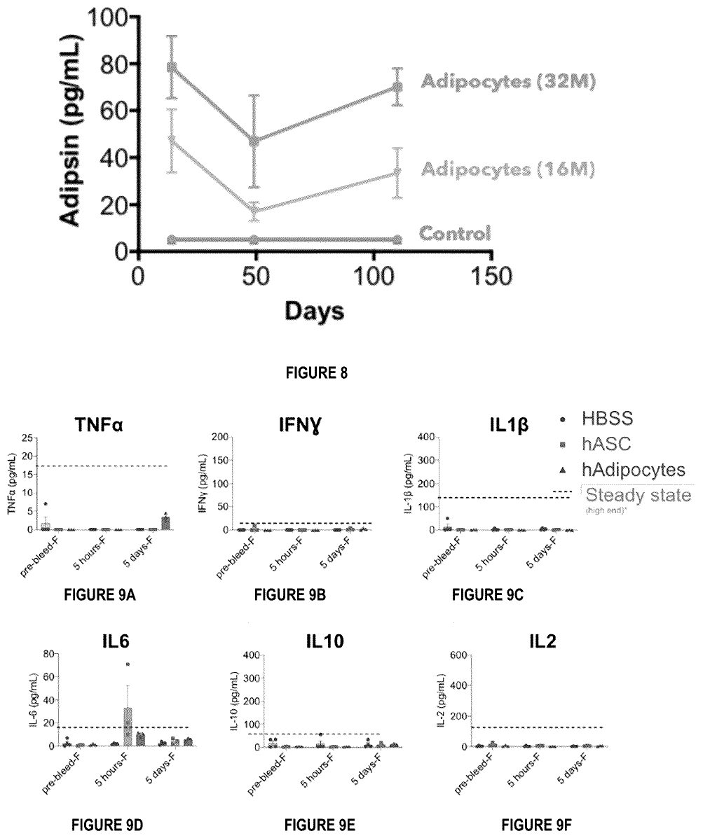

hAdipocyte engraftment was demonstrated by detection of human Adipsin levels in plasma. The level of human adipsin secretion was analyzed in serum using the cell ELISA kits for human adipsin (LEGENDplex™ Human Adipokine, Biolegend) up until 126 days post administration. Human adipsin level was detected in plasma up to 126 days post transplantation as shown in FIG. 8. Human adipsin was detected at higher levels ˜80 pg/ml when 32M human cells were dosed compared to ˜50 pg/ml when 16M human cells were dosed, furthermore a very low background level of ˜5 pg/ml was found in control mice dosed with HBSS.

In summary, the results from this example show, inter alia, that adipocytes derived from human ASCs in culture can be transplanted to achieve long-lasting adipocyte engraftment in vivo. This example also demonstrates, inter alia, the ability to achieve long-term in vivo secretion of human adiponectin from transplanted human adipocytes.

### Example 8A: Non-Immunogenicity of ASCs and Differentiated Adipocytes in Culture

This example demonstrates, inter alia, that ASCs and adipocytes derived from ASCs or CD34⁺ cells in culture have low immunogenicity.

In this example, hASCs are isolated and expanded as described in Example 1. hAdipocytes are derived from hASCs as described in Example 3 or from human CD34⁺ cells as described in Example 4. The immunogenic properties of both of these cell types are assessed using immunophenotyping or the one-way mixed lymphocyte reaction (MLR) assay.

For immunotyping, the cells are characterized for immunogenic markers using flow cytometric analysis. Human peripheral blood mononuclear cells (hPBMCs) (AllCells) are used as a control. The cells are washed with PBS containing 1% FBS, incubated with a directly conjugated antibody against MHC class I (HLA-ABC), MHC class II (HLA-DR), CD40, CD80, or CD86 (all from Biolegend) for 30 minutes at 4° C. The cells are then washed and analyzed with a flow cytometer. Isotype-match negative controls are used to define the background staining. hASCs and hAdipocytes are expected to express lower levels of MHC class I, MHC class II, CD40, CD80, and CD86 compared to hPBMCs.

The immunogenicity of hASCs and hAdipocytes are also characterized using the one-way MLR assay. The responder cells in the MLR assay are prepared as follows. hPBMCs are prepared by centrifugation of leukopheresed peripheral blood cells (AllCells) over an LSM density gradient. T cells are purified from a portion of the PBMCs by negative selection using magnetic beads. Briefly, hPBMCs are treated with a cocktail of monoclonal antibodies (mAbs) (all from Serotec) chosen to bind to monocytes (anti-CD14; clone UCHM1), B cells (anti-CD19; clone LT19), natural killer cells (anti-CD56; clone ERIC-1), and cells expressing MHC class II antigens (anti-MHC class II DR; clone HL-39). hPBMCs are mixed with magnetic beads coated with anti-mouse IgG antibody (Dynal Corp). Bead-bound cells are removed using a magnet, leaving a population of purified T cells (>90% T cells by flow cytometry using anti-CD3 mAb).

The purified responder T cells are labeled with 5,6-carboxyfluorescein diacetate succinimidyl ester (CFSE) (CellTrace™ CFSE, ThermoFisher, C34554) to track cell proliferation. Specifically, the cells are pelleted, gently resuspended in CellTrace™ CFSE staining solution (1:1000 dilution), and incubated at 37° C. for 20 minutes, protected from light. Next, five times the original staining volume of culture medium is added to the cells and incubated for 5 min. The cells are then pelleted and resuspended in fresh warm culture medium. The culture medium is Iscove's modified Dulbecco's medium supplemented with sodium pyruvate, nonessential amino acids, antibiotics/antimycotics, 2-mercaptoethanol (all reagents from Gibco), and 5% human AB serum (Pel-Freez).

The MLR is performed in 96-well microtiter plates. CFSE-labeled purified T cells derived from two different donors are plated at 2×10⁵ cells per donor per well. Different donors are used to maximize the chance that at least one of the T cell populations is a major mismatch to the hASCs and hAdipocytes. Stimulator cells used in the assay include autologous hPBMCs (baseline response), allogeneic hPBMCs (positive-control response), hASCs, and hAdipocytes. The hASCs and hAdipocytes are pretreated with 50 μg/mL mitomycin C (MMC) at 37° C. for 3 hours, and hPBMCs are pretreated with the same dose for 30 min. Additional control cultures consist of T cells plated in medium alone (no stimulator cells). Triplicate cultures are performed for each treatment. Stimulator cells are then added to the culture wells at various numbers, ranging from 5,000 to 20,000 cells per well. After 3 days of incubation, the supernatants are collected and analyzed to determine the levels of the proinflammatory cytokines interferon γ (IFN-γ) and tumor necrosis factor α (TNF-α) through enzyme-linked immunosorbent assay (R&D Systems). The proliferation in the remaining T cells is analyzed using a flow cytometer with 488-nm excitation and a 530/30-nm bandpass emission filter for CellTrace™ CFSE staining. The discrete peaks in the histogram represent successive generations of the proliferating cells. The relative numbers of T-cell precursors required for generating these daughter cells under each division peak is calculated by dividing the number of daughter-cell events by 2 raised to the power of the given round of division (2ⁿ). The sum of all the calculated numbers of precursors from each division peak is used to represent the number of reactive T-cell precursors.

The immune response is evaluated based on the proliferation of purified responder T cells and the secretion of IFN-γ and TNF-α. It is anticipated that the proliferation of the responder cells increases significantly when they are cocultured with allogeneic hPBMCs. In contrast, no significant proliferation of the responder cells is expected in coculture with hASCs or hAdipocytes. In addition, a significant increase in IFN-γ and TNF-α secretion should be observed in coculture with allogeneic hPBMCs while no significant secretion is expected in coculture with hASCs or hAdipocytes.

In conclusion, the results in this example show, inter alia, that hASCs and culture-derived hAdipocytes are non-immunogenic, as demonstrated in the low expression levels of immunogenic markers and the lack of an immune response when cocultured with allogeneic T-cells.

### Example 8B: Non-Immunogenicity of ASCs and Differentiated Adipocytes in Culture

This example demonstrates, inter alia, that ASCs and adipocytes derived from ASCs cells in culture do not induce an innate immune response after cellular transplantation.

In this example, hASCs were expanded as described in Example 5A and/or 5B, and adipocytes are generated as described in Example 7A and/or 7B. After determining the total cell count, the cells were pelleted by centrifuging in a swinging bucket centrifuge at 80×g for 5 minutes. After pelleting, supernatant was aspirated off and ASCs and adipocytes were resuspended separately in pre-chilled HBSS at a concentration of 4×10⁶/100 uL each.

The immunogenic properties of both of these cell types were assessed by transplanting into immunocompetent animals and assessing cytokine levels in plasma before transplantation, and 5-hours and 5 days post transplantation. C57BL/6j were injected with either hASCs or adipocytes derived from hASCs (hAdipocytes). The dorsal side of each mouse was swabbed with 70% ethanol, and the ASCs and adipocytes suspended in HBSS (4×10⁶ cells/side) were injected using a 25G gauge syringe into the side of the dorsal flank for subcutaneous dosing. In the mock-transplanted cohort, an equal volume of HBSS alone iwa injected. Post recovery, the mice were fed a high fat diet (Research Diets, D1245145% high fat diet product #NC9248609) for 28 days followed by normal chow diet (LabDiet, 5001) for the remainder of the study. At both 5 hrs post administration as well as 5 days post administration hASCs and hAdipocytes did not induce an immune response in immune competent murine animals as shown by the expression of TNFalpha, IFNy, IL1B, IL6, IL10 and IL-2 (FIGS. 9A-9F).

For immunophenotyping of hASCs and hAdipocytes derived from hASCs immunogenic and cell type specific surface markers were evaluated using flow cytometry. The cells were harvested from cell culture vessels using trypsin and washed with HBSS containing 3% FBS, 10 mM EDTA. 0.1×10⁶ to 1×10⁶ cells are incubated with a directly conjugated antibody against MHC class I (HLA-ABC), MHC class II (HLA-DR), CD40, CD80, CD45, and CD90 (all from Biolegend) for 30 minutes at 4° C. The cells were then washed and analyzed with an Attune NXT flow cytometer.

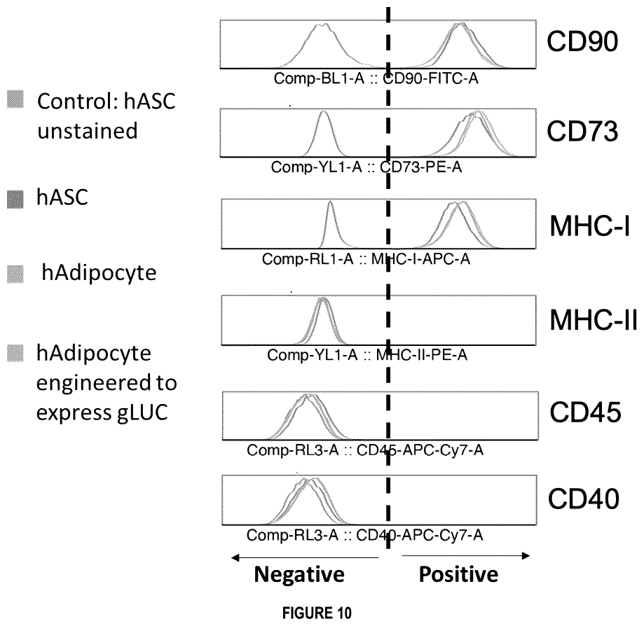

Cytokine assessment was performed on mouse plasma or serum. For plasma, mouse blood was collected into EDTA-coated tubes and processed by centrifuging at 3,000×g for 10 minutes at 4° C. Plasma was aliquoted and diluted 2-fold with PBS pH˜7.5 prior to freezing at −80° C. Cytokines in plasma were assessed in duplicate measurements on a Mouse Cytokine Array Proinflammatory Focused 10-plex (MDF10) from Eve Technologies Corporation (Calgary, AB Canada). Both ASCs and adipocytes were positive for CD90, CD73 and MHC-I, while negative for MHC-II, CD45 and CD40 (FIG. 10).

In conclusion, the results in this example show, inter alia, that hASCs and culture-derived hAdipocytes were non-immunogenic, as demonstrated in the low expression levels of immunogenic markers on the cells, as well as no induction of an immune-response after transplantation in immune-competent animals.

### Example 8C: Long-Term Engraftment of Xenografted Human Adipose Cells in Immune Competent Mice (In Vivo)

This example demonstrates, inter alia, the ability of transplanted human adipose cells to be dosed without inducing a substantial immune response in immune competent animals. Human adipocytes survive in vivo in immune competent mice as demonstrated, inter alia, by the detection of adipogenic grafts at site of implantation 92 days after administration.

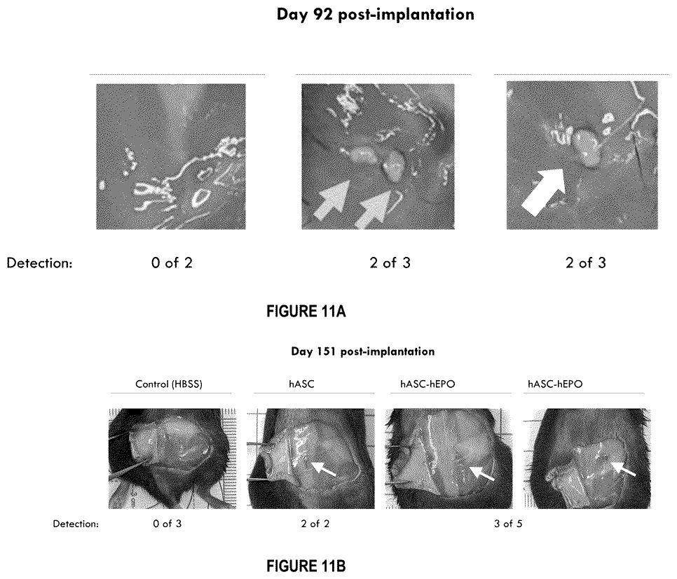

In this example, C57BL/6J mice dosed with human ASCs and adipocytes, as described in Example 8A, were followed over time. Animals were euthanized 92 days after transplantation and implantation sites were analyzed. As shown in FIG. 11A, adipose grafts were detected in animals dosed with ASCs (2 of 3) and adipocytes (2 of 3) but not in control animals (0 of 2).

Human cell implantation in vivo was monitored via visible graft at day 92 post-transplant in the dorsal flank. Overall, the example demonstrates that human ASCs and human adipocytes do not induce a substantial immune response and persist for more than 92 days in recipient mice.

In another cohort hASC that were genetically modified to express EPO under an EF1a promoter were also analyzed for xenograft survival in immune-competent animals. In this example, hASCs were expanded as described in Example 5A and/or 5B. Once cells reached 70% confluence, they were passaged as described in Example 5A and/or 5B and seeded into 6 well culture plates at 1×10⁵ cells/well and allowed to culture overnight. The following day, cells were transfected with a pre-determined MOI, with a lentivirus reporter vectors expressing a human EPO (hEPO) reporter gene with a puromycin resistance gene. hASCs were subsequently expanded as described in Example 5A and/or 5B. Unengineered hASCs (8×10⁶ cells/side) and engineered hASCs (16×10⁶ cells/side) were transplanted into mice as described previously. In short C57BL/6J mice were injected with ASCs. The dorsal side of each mouse was swabbed with 70% ethanol, and the ASCs suspended in HBSS were injected using a 25G gauge syringe into the side of the dorsal flank for subcutaneous dosing. In the mock-transplanted cohort, an equal volume of HBSS alone. Post recovery, the mice were fed a high fat diet (Research Diets, D1245145% high fat diet product #NC9248609) for 28 days followed by normal chow diet (LabDiet, 5001) for the remainder of the study.

Animals were euthanized 151 days after transplantation and implantation sites were analyzed for the presence of an adipose vascular graft. As shown in FIG. 11B, adipose grafts were detected in animals dosed with ASCs (2 of 2) and ASC-hEPO (3 of 5) but not in control animals (0 of 3).

Overall, the example demonstrates, inter alia, that human ASCs and human adipocytes do not induce a substantial immune response and persist for more than 92 days in recipient mice.

### Example 9: Engineering GFP-Expressing ASCs or CD34⁺ Cells that Express Firefly Luciferase Upon Differentiation into Adipocytes

This example demonstrates, inter alia, the ability to genetically engineer ASCs or CD34⁺ cells to express GFP constitutively and upon differentiation into adipocytes express firefly luciferase.

In this example, ASCs and CD34⁺ cells are isolated and expanded as described in Examples 1 and 2. The cells are either from human origin (hASCs and hCD34⁺ cells) or murine origin (mASCs). The cells are genetically labeled with two lentivirus reporter vectors expressing a green fluorescent protein (GFP) reporter gene (SEQ ID NO: 1) and a firefly luciferase (Luc) reporter gene (SEQ ID NO: 2). GFP expression is driven by the constitutive promoter CMV (pCMV) (SEQ ID NO: 3) and is used to identify transplanted cells. Luc expression is driven by the human adiponectin promoter (phAdipoQ) (SEQ ID NO: 4) in hASCs and hCD34⁺ cells or the murine adiponectin promoter (pmAdipoQ) (SEQ ID NO: 5) in mASCs. See Segawa et al., J. Endocrinol. 200, 1, 107-116 and Koshiishi et al., Gene 424, 1-2, 146, both of which are incorporated by reference herein in their entireties. The adiponectin promoters drive adipocyte-specific expression of the firefly luciferase reporter, which is used to identify adipocytes derived from the transplanted cells in situ.

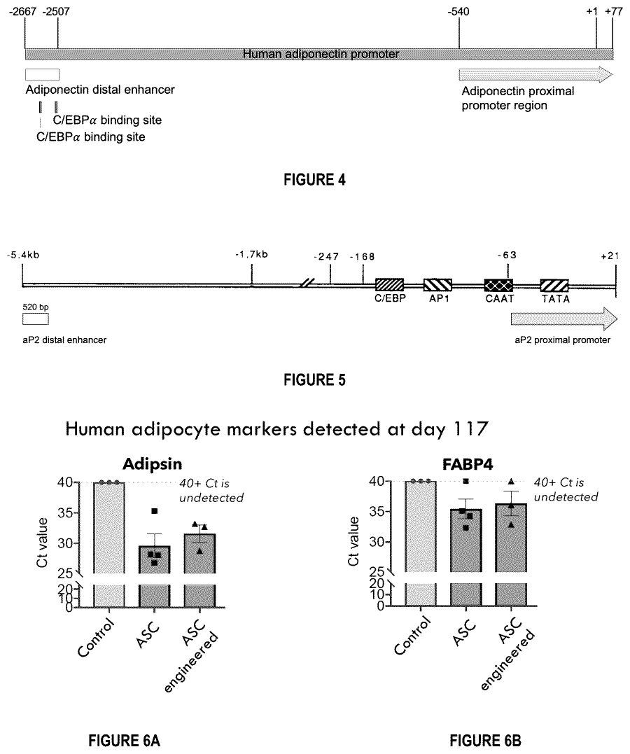

The human adiponectin promoter contains minimally a distal enhancer region (−2667 to −2507 bp upstream from human adiponectin's transcriptional start site) and a proximal promoter region (−540 to +77 bp from human adiponectin's transcriptional start site) (Segawa et al, 2009) (FIG. 4). The distal enhancer is highly conserved and contains two completely conserved CCAAT boxes. The transcription factor CCAAT/enhancer-binding protein α (C/EBP α) binds to this enhancer and regulates the transcriptional activity of adiponectin gene. The proximal promoter region is found to be necessary for full transcriptional activation by its distal enhancer.

The murine adiponectin promoter also contains a distal enhancer region (−2228 to −2066 bp upstream from murine adiponectin's transcriptional start site) necessary for full transcriptional activation (Koshiishi et al, 2008). The distal enhancer contains two conserved motifs CACAATGC that are bound by transcription factors C/EBPα, C/EBPβ, and C/EBPδ.

Alternative promoters can also be used to drive adipocyte-specific trans-gene expressions. An example is the aP2/FABP4 promoter (SEQ ID NO: 13). The aP2/FABP4 minimal promoter contains a 518-bp enhancer fragment mapping between kb −4.9 and kb −5.4 (upstream from aP2's transcriptional start site) and a proximal promoter region (−63 to +21 bp from murine aP2's transcriptional start site) (FIG. 5) See Graves et al, J. Cell. Biochem. 49, 219-244 (1992), which is incorporated by reference herein in its entirety.

The HIV-1 based lentivirus is constructed and produced using a third-generation packaging system See Dull et al., J. Virol. 72, 11, 8463-8471 (1998), which is incorporated by reference herein in its entirety. The system consists of four plasmids, the plasmid of interest, two helper plasmids (package), and a plasmid encoding the envelope (VSV-G glycoprotein). In one lentivirus (LV-71.1), the plasmid of interest encodes the GFP protein under the control of the CMV promoter (pCMV-GFP) and expresses a Hygromycin B resistance gene (SEQ ID NO: 6) as a selection marker. In another lentivirus, the plasmid of interest encodes the firefly luciferase protein under the control of the hAdipoQ (phAdipoQ-Luc in LV-71.3) or mAdipoQ promoter (pmAdipoQ-Luc in LV-71.6) and expresses a Puromycin resistance gene (SEQ ID NO: 7) as a selection marker.

The lentiviruses are generated using the 293T cells and the pPACKH1 packaging kit (System Biosciences, LV500A). Briefly, 18-24 hours prior to transfection, 293T cells are seeded in 150 cm² plate at a density of 7-8×10⁶ cells in 20 mL DMEM with high-glucose (Gibco, 11965084) supplemented with 10% FBS, GlutaMAX™ (Gibco, 35050061), and penicillin-streptomycin. To prepare the transfection mixture, 45 μL of pPACKH1, 5-8 μg of the plasmid of interest, and 55 μL of PureFection™ transfection reagent (System Biosciences, LV750A) are added to each 1 mL of serum-free DMEM. The mixture is incubated at room temperature for 15 minutes and then added dropwise into the 293T cell culture plate. The plate is returned to the cell culture incubator at 37° C. with humidified 5% CO₂. The medium containing lentiviruses is collected at 48 hours and 72 hours after transfection. The medium is centrifuged at 3,000×g for 15 minutes at room temperature to pellet cell debris. The supernatant containing viral particles is collected. In order to concentrate the viruses, 1 volume of cold PED-it Virus Precipitation Solution (System Biosciences, LV810A) is added every 4 volumes of the supernatant. The mixture is then incubated at 4° C. for at least 12 hours and centrifuged at 1,500×g for 30 minutes at 4° C. The supernatant is removed, and the pellet containing lentiviral particles is resuspended in 1/10 to 1/100 of original volume using cold PBS. The viral suspension is frozen and stored at −80° C. until ready for use.

The ASCs or CD34⁺ cells are transduced with lentiviral vectors as follows. The transduction is performed in 24-well microtiter plates. The cells are plated at a density of 5×10⁴ cells/well. Transduction is performed when the cells are between 50 to 70% confluent. The transduction medium is the complete culture medium supplemented with TransDux™ (System Biosciences, LV860A) at 1:200 dilution or 4 μg/mL protamine sulfate (Fisher, ICN10275205). A mixture of either LV-71.1 and LV-71.3 (for hASCs and human CD34⁺ cells) or LV-71.1 and LV-71.6 (for mASCs) is combined with the transduction medium and then added to each well at varying MOIs (20-140). After 72 hours of transduction, the medium is aspirated off, and fresh medium is added to each well. The cells are examined for GFP expression using an epifluorescence microscope. To establish stable cell lines, the cells are selected in culture medium containing Hygromycin B (50-200 μg/mL) (Fisher, 40005220ML) and Puromycin (1-5 μg/mL) (Fisher, 50-165-7050). Culture medium with fresh antibiotics is replaced every 3-4 days until resistant colonies can be identified, which is typically after 10-14 days of selection.

The expressions of the reporter proteins are characterized in the genetically modified cells as follows. The ASCs and CD34⁺ cells are differentiated into adipocytes as described in Examples 3 and 4, respectively. GFP expression is assessed with flow cytometric analysis or fluorescence microscopy. It is expected that GFP is highly expressed in ASCs, CD34⁺ cells, and adipocytes. Firefly luciferase activity is quantified using a standard luciferase assay (Promega, E1500). Briefly, cells are lysed with Cell Culture Lysis Reagent (Promega, E1531). The cell lysate is then mixed with Luciferase Assay Reagent containing beetle luciferin (Promega, E1483), and the luminescence from the reaction is measured using a luminometer. Adipocytes are expected to exhibit higher luciferase activity compared to ASCs and CD34⁺ cells.

Overall, this example demonstrates, inter alia, the ability to engineer ASCs and CD34⁺ cells to constitutively express GFP and produce adipocytes that express both GFP and firefly luciferase.

### Example 10: Biodistribution of Adipocytes Derived from Transplanted ASCs or CD34⁺ Cells

This example demonstrates, inter alia, the ability to control and measure the biodistribution of adipocytes derived from transplanted ASCs or CD34⁺ cells.

In this example, ASCs and CD34⁺ cells are genetically modified as described in Example 9 and transplanted into mice as described in Examples 5 and 6, respectively. The biodistribution of adipocytes derived from the transplanted cells is assessed using whole-body imaging of luciferase activity every week up to six months post recovery. Specifically, luciferase activity is measured in transplant-naïve mice and mice transplanted with ASCs or CD34⁺ cells in an IVIS Imaging System 50 (Caliper Life Sciences, Hopkinton, MA, USA). Animals are lightly anesthetized with pentobarbital (65 mg/kg, i.p.) and injected with D-luciferin (120 mg/kg, 100 μL retro-orbital). Measurements are initiated 3 min after luciferin injection, and luminescence is integrated over 5 min.

In addition, at 2, 4, and 6 months post recovery, mice are euthanized immediately after whole-body imaging. Mouse adipose depots (gonadal, perirenal, retroperitoneal, mesenteric, and inguinal) and non-adipose depots (lower hind limb skeletal muscle, liver, and lung) are harvested. Additional harvested sites are the grafted tissues or bone marrow in mice transplanted with ASCs or CD34⁺ cells, respectively. The harvested tissues are quickly returned to the imager for analysis of isolated tissue luminescence. The tissues are then minced into ˜4 mm³ pieces and fixed in 1% paraformaldehyde for 15 min at room temperature. The fixed tissues are rehydrated in PBS 3×10 min each and stained DAPI (ThermoFisher, D1306) (1 μg/mL, to visualize the nuclei) and anti-GFP antibody (to locate the transplanted cells) (Biolegend). The stained tissues are then washed and imaged in an EVOS M5000 imaging system (ThermoFisher) using the 20× objective.

In the mice transplanted with ASCs, it is expected that light emission will be detected in the grafted sites as early as 2-4 weeks post engraftment via both whole-body imaging, indicating the appearance of differentiated adipocytes. Trace amount of light emission may also be observed outside of the grafted sites due to migration of ASCs. Light emission will increase over time as the number of differentiated adipocytes increases. Luciferase activity from the harvested tissues is expected to be consistent with the in vivo imaging results. Specifically, luciferase activity will be at high levels in the grafted tissues, and a small amount of light emission is anticipated in mouse adipose depots outside of the grafted tissues. At least 50% of the transplanted GFP⁺ cells are expected to be present in the grafted sites throughout the length of the study, demonstrating that transplanted ASCs achieve long-lasting engraftment. GFP⁺ cells may also be detected in tissues outside of the grafted sites as further evidence of ASC migration.

In mice transplanted with CD34⁺ cells, significant whole-body light emission is expected to appear 4-8 weeks after transplant and will increase over time, demonstrating that the transplant-derived adipocytes are distributed throughout the body. In harvested tissues, luciferase activity is expected to be at high levels in all adipose tissues while not significantly above baseline in non-adipose tissues. The presence of the transplanted CD34⁺ cells will be detected as GFP⁺ cells in varying numbers in most harvested tissues, including bone marrow, adipose tissues, and non-adipose tissues throughout the length of the study.

The results from this example are expected to demonstrate, inter alia, that biodistribution of adipocytes derived from transplanted ASCs or CD34⁺ cells can be controlled and measured. Specifically, localized distribution of adipocytes via local ASC injection is expected. Additionally, widespread adipocyte distribution throughout the body is expected via systemic injection of CD34⁺ cells.

### Example 11A: Biodistribution of Transplanted Adipocytes

This example demonstrates, inter alia, the ability to control and measure the distribution of transplanted adipocytes.

In this example, ASCs and CD34⁺ cells are genetically modified as described in Example 9 and differentiated into adipocytes in vitro as described in Examples 3 and 4, respectively. The genetically labeled adipocytes are transplanted into mice as described in Example 7A and/or 7B. The biodistribution of adipocytes derived from the transplanted cells is assessed using whole-body imaging of luciferase activity every week up to six months post recovery. Specifically, luciferase activity is measured in transplant-naïve mice and mice transplanted with adipocytes in an IVIS Imaging System 50 (Caliper Life Sciences, Hopkinton, MA, USA) as described in Example 10. In addition, at 2, 4, and 6 months post recovery, mice are euthanized immediately after whole-body imaging. The grafted tissues, recipient mouse adipose depots (gonadal, perirenal, retroperitoneal, mesenteric, and inguinal), and non-adipose depots (lower hind limb skeletal muscle, liver, and lung) are harvested. The harvested tissues are analyzed for luminescence and GFP⁺ cells as described in Example 10.

It is expected that light emission in whole-body imaging will be detected mainly at the transplanted sites as early as 1 week after injection and will persist up to 6 months. Harvested tissues at the grafted sites will also exhibit high levels of luciferase activity. In contrast, there will be no significant light emission in harvested tissues outside of the grafted sites. More than 50% of the GFP⁺ cells at the grafted sites are expected to persist throughout the length of the study.

Overall, results from this example demonstrate, inter alia, that it is possible to locally engraft long-lasting adipocytes.

### Example 11B: Biodistribution of Transplanted Adipocytes

This example demonstrates, inter alia, the ability to track the distribution of transplanted adipocytes and demonstrates the longevity of adipocytes after transplantation.

In this example, ASCs and hAdipocytes derived from ASCs were genetically modified as described in Example 9 and differentiated into adipocytes in vitro as described in Example 7A and/or 7B, respectively. The genetically labeled adipocytes were transplanted at two doses, 2 million and 8 million, subcutaneously into mice.

NOD SCID mice (The Jackson Laboratory, 001303) were injected with adipocytes derived from hASCs. The dorsal side of each mouse was swabbed with 70% ethanol, and the adipocytes suspended in HBSS (2 or 8×10⁶ cells/side) were injected using a 25G gauge syringe into the side of the dorsal flank for subcutaneous dosing. In the mock-transplanted cohort, an equal volume of HBSS alone was injected. Post recovery, the mice were fed a high fat diet (Research Diets, D1245145% high fat diet product #NC9248609) for 28 days followed by normal chow diet (LabDiet, 5001) for the remainder of the study.

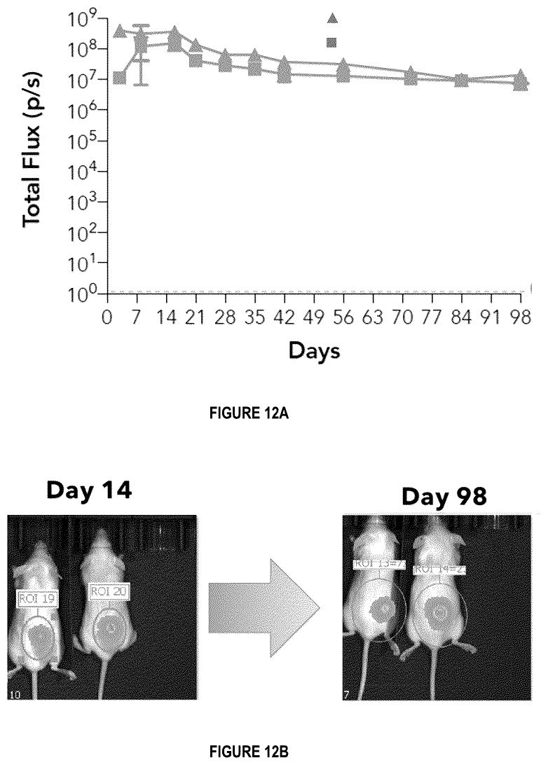

The biodistribution of adipocytes derived from the transplanted cells was assessed using whole-body imaging of luciferase activity from day 3 until day 98 post administration. Specifically, Firefly luciferase activity was measured in transplant-naïve mice and mice transplanted with adipocytes in an IVIS Lumina LT Series 3 Caliper Life Sciences, Hopkinton, MA, USA). Luciferase was analyzed from day 3-day 98 post transplantation and was detected at all timepoints (FIG. 12A). Furthermore, the implant did stay localized around the injection site for 98 days (FIG. 12B).

Overall, results from this example demonstrate, inter alia, that it was possible to locally engraft long-lasting adipocytes.

### Example 12: Therapeutic Effects in a Zellweger Mouse Model by Transplanting Unmodified Adipogenic Cells

This example demonstrates, inter alia, that transplanting unmodified adipogenic cells alleviates pathogenic phenotypes of a Pex5^{-/-} Zellweger mouse model.

In this example, Pex5^{-/-} mice on a C57BL6/J genetic background are generated by mating Pex5-loxP mice (The Jackson Laboratory, 031665) with Nestin-Cre mice (The Jackson Laboratory, 003771). mASCs from wild-type C57BL6/J mice are isolated and expanded as described in Example 1. Murine adipocytes are derived from the mASCs in culture as described in Example 3. The mASCs or murine adipocytes are suspended in PBS solution at 5-10×10⁶ cells/mL, and 10 μL of the cell suspension is injected using a 26G gauge syringe into each side of the dorsal flank of newborn Pex5^{-/-} or wild-type pups. In the control cohort, Pex5^{-/-} or wild-type pups are injected in the same manner with 10 μL PBS. Total body weight is monitored every day up to 2 weeks after birth. On days 2, 3, 7, and 14, liver, kidney, brain, and fat tissues are harvested and weighed.

It is expected that at least 20% of the Pex5^{-/-} pups transplanted with wild-type mASCs or wild-type murine adipocytes will survive more than 3 days and up to 2 weeks after birth whereas all control Pex5^{-/-} pups will die at various time before 3 days. Furthermore, after transplantation, Pex5^{-/-} pups will start increasing in total body weight compared to the age-matched control Pex5^{-/-} pups. The harvested tissues of the transplanted Pex5^{-/-} pups will also weigh significantly higher compared to those of the age-matched control Pex5^{-/-} pups. Finally, the severe physiological distress behavior typically observed in Pex5^{-/-} pups (e.g. inability to support body weight on legs, gasping, compensatory abdominal breathing, and periods of apnea) is expected to be less pronounced in the age-matched transplanted Pex5^{-/-} pups.

Overall, this example shows that unmodified wild-type adipogenic cells (ASCs and derived adipocytes) are able to promote survival and reduce symptoms in a Zellweger disease mouse model upon transplantation into newborn pups.

### Example 13: Identification and Isolation of Highly Adipogenic ASCs

This example demonstrates, inter alia, that a subtype of ASCs that are highly adipogenic can be identified and isolated.

In this example, an ASC subtype that is the strongest responder to adipogenic differentiation was identified using RNA sequencing data from Min et al., PNAS 116, 36, 17970-17979 (2019), which is incorporated by reference herein in its entirety. Specifically, using k-means clustering on 52 clonal ASC populations that underwent adipogenic differentiation, a cluster of 13 populations that show high expression levels across 10 adipocyte-specific genes (CIDEC, FABP4, PLIN1, LGALS12, ADIPOQ, TUSC5, SLC19A3, PPARG, LEP, CEBPA) was identified. See Ahn et al., Sci. Rep. 9, 1, 3087 (2019), which is incorporated by reference herein in its entirety. The 13 ASC clones that give rise to these populations are the strongest responders of adipogenic differentiation. In order to experimentally isolate these ASCs, a set of cell surface proteins that are differentially expressed between them and the remaining ASC clones was identified. The 4 most upregulated genes for the strongest responders are CDw210, CD107b, CD164, and CD253, and the 4 most downregulated genes are CD266, CD151, CD49c, and CD91.

hASCs are isolated and expanded as described in Example 1. The single-cell suspension is diluted to 0.75 or 1×10⁷ cells/ml with FACS buffer (PBS with 3% FBS, 1 mM EDTA, 1% penicillin-streptavidin) and stained with directly conjugated antibodies against CDw210, CD107b, CD164, CD253, CD266, CD151, CD49c, and CD91. The cells are incubated with the cocktail of antibodies on ice for 20 min protected from light, after which they are washed and stained with DAPI (Sigma #D9542) or propidium iodide (Molecular Probes #P3566) for assessing viability and subjected to FACS using a Becton Dickinson FACSAria II sorter. Compensation measurements are performed for single stains using compensation beads (eBiosciences #01-2222-42). The following gating strategy is applied while sorting the cells: first, the cells are selected based on their size and granulosity or complexity (side and forward scatter), and then any events that could represent more than one cell are eliminated. Next, the CD266⁻ CD151⁻ CD49⁻ CD91⁻ population is selected and is used to select for populations that are positive for one or a combination of the following markers: CDw210, CD107b, CD164, and CD253.

Each of the selected populations is tested for adipogenicity in vitro. The pre-selected ASC population is used as a control. The cells are subjected to the in vitro adipogenic differentiation procedure as described in Example 3. Adipogenic differentiation is measured after 3, 7, and 14 days in adipogenic induction medium via Oil Red O staining, LipidTox staining, and qPCR of adipogenic markers as described in Example 3. It is expected that one or more of the selected ASC populations will yield significantly more adipocytes than the control at one or more of the time points as measured by Oil Red O and LipidTox staining. In addition, one or more of these populations will achieve >80% as early as 3 days in adipogenic induction medium. Finally, it is expected that one or more of the selected populations will express one or more of the adipogenic markers at significantly higher levels compared to the control upon differentiation.

The selected ASC populations are also tested for their capacity to generate adipocytes in vivo. The pre-selected ASC population is also used as a control. The ASC populations are transplanted into mice, and the presence of the derived adipocytes is measured as the serum level of human adiponectin as described in Example 5A and/or 5B. It is expected that one or more of the selected ASC populations will lead to a significantly higher serum level of human adiponectin compared to the control ASC population as early as 14 days post transplantation.

Overall, this example demonstrates, inter alia, that an ASC subtype can be identified that is highly adipogenic and can be used to efficiently produce adipocytes in vitro and in vivo.

### Example 14A: In Vitro Isolation, Characterization, and/or Modulation of ASCs for Adipocytes Highly Secreting Adiponectin

This example demonstrates, inter alia, that a subtype of ASCs that produce adipocytes secreting high levels of adiponectin can be identify and isolate.

In this example, we identified an ASC subtype that is the highest producer of adiponectin using RNA sequencing data from Min et al., PNAS 116, 36, 17970-17979 (2019), which is incorporated by reference herein in its entirety. Specifically, among the strongest adipogenic responders identified in Example 13, we identified a cluster of 8 ASC clones that give rise to adipocytes expressing 2.5-10 times more adiponectin than average. In order to experimentally isolate these ASCs, we identified plasma membrane proteins that are differentially expressed between them and the remaining ASC clones. The 4 most upregulated genes are CD361, CD120b, CD164, and CD213A1, and the 4 most downregulated genes are CD266, CD167, CD325, and CD115.

hASCs are isolated and expanded as described in Example 1. Using FACS as described in Example 13, cell populations that are negative for the markers CD266, CD167, CD325, and CD115 and positive for one or a combination of the markers CD361, CD120b, CD164, and CD213A1 is selected.

The selected ASC populations are differentiated into adipocytes in vitro as described in Example 3. The derived adipocytes are tested for adiponectin secretion in vitro. The adipocytes derived from the pre-selected ASC population are used as a control. The number of differentiated adipocytes is measured using Oil Red O or LipidTOX staining as described in Example 3. The level of adiponectin secretion per adipocyte is calculated by collecting and analyzing the cell culture supernatants using an ELISA kit for human adiponectin (Zen-Bio, Inc., ADIP-1) and normalized by the number of differentiated adipocytes for each sample. It is expected that one or more of the selected ASC populations will produce adipocytes that secrete significantly higher levels of adiponectin compared to the control.

The adipocytes derived from the selected ASC populations are transplanted into mice in order to test for their adiponectin secretion capacity in vivo. The same number of adipocytes derived from the control ASC population is also transplanted. The transplantation procedure is described in Example 7A and/or 7B. The serum level of human adiponectin is measured at different time points also as described in Example 7A and/or 7B. It is expected that significantly higher levels of human adiponectin will be produced by the adipocytes derived from the selected ASC populations compared to the control.

In summary, this example shows, inter alia, that an ASC subtype that can be used to derive adipocytes secreting high levels of adiponectin can be identified and isolated.

### Example 14B: In Vitro Isolation, Characterization, and/or Modulation of ASCs for Adipocytes Highly Secreting Adiponectin

This example demonstrates, inter alia, that a subtype of ASCs can be identified and isolated which can differentiate into adipocytes that secrete high levels of adiponectin.

hASCs were immunophenotyped and cell surface proteins that displayed heterogeneous expression were identified. hASCs were isolated and expanded as described in Example 5A and/or 5B. Using FACS as described in Example 8A, cell populations that are positive and negative for the CD10 marker were sorted into separate wells. Unstained control cells were sorted into separate wells.

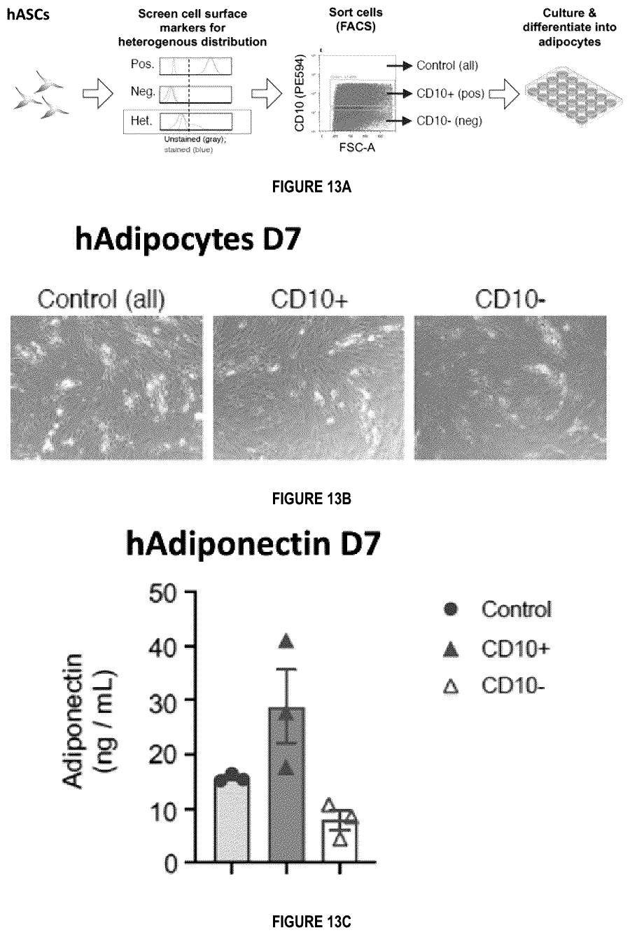

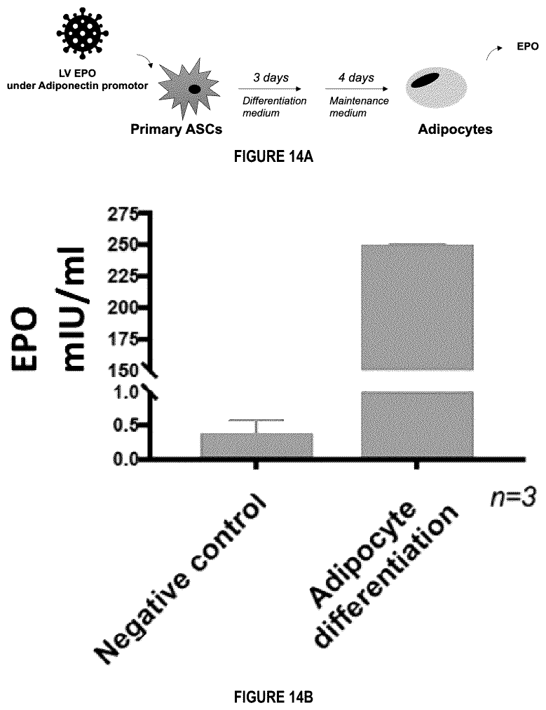

The selected ASC populations were differentiated into adipocytes in vitro as described in Example 7A and/or 7B. The derived adipocytes were tested for adiponectin secretion in vitro using an ELISA kit. The CD10+ selected ASC populations produced adipocytes that secrete significantly higher levels of adiponectin compared to the control and CD10− (FIGS. 13A-13C).

In summary, this example shows, inter alia, that a CD10+ ASC subtype can be used to derive adipocytes secreting high levels of adiponectin and can be identified and isolated.

### Example 15: In Vitro Isolation, Characterization, and/or Modulation of ASCs for Adipocytes Highly Expressing PEX5

This example demonstrates, inter alia, that a subtype of ASCs that produce adipocytes expressing high levels of intracellular PEX5 can be identified and isolated.

In this example, an ASC subtype was identified that is the highest producer of PEX5 using RNA sequencing data from Min et al., PNAS 116, 36, 17970-17979 (2019), which is incorporated by reference herein in its entirety. Specifically, among the strongest adipogenic responders identified in Example 13, a cluster of 3 ASC clones was identified that give rise to adipocytes expressing PEX5 at levels higher than 75% of the population. In order to experimentally isolate these ASCs, we identified plasma membrane proteins that are differentially expressed between them and the remaining ASC clones. The 3 most upregulated genes are CDw210b, CD340 and CDw293, and the 4 most downregulated genes are CD151, CD10, CD26, and CD142.

hASCs are isolated and expanded as described in Example 1. Using FACS as described in Example 13, cell populations that are negative for the markers CD151, CD10, CD26, and CD142 and positive for one or a combination of the markers CDw210b, CD340 and CDw293 are selected.

The selected ASC populations are differentiated into adipocytes in vitro as described in Example 3. The derived adipocytes are tested for PEX5 gene expression via qPCR. The adipocytes derived from the pre-selected ASC population are used as a control. qPCR is performed as described in Example 3. The qPCR primers for human PEX5 are 29 and 30. GAPDH (primers 21 and 22) and actin (primers 25 and 26) are used as controls. It is expected that adipocytes derived from one or more of the selected ASC populations will show significantly higher PEX5 gene expression levels compared to the control.

PEX5 protein expression is measured using Western blot analysis. Total proteins from differentiated adipocytes in a 12-well plate are harvested by adding 200 μL of RIPA buffer onto each well. Next, 10 μg of cell lysate proteins are analyzed on 10-20% gradient polyacrylamide/SDS gel. After electrotransfer to a nitrocellulose membrane using dry transfer method, the blot is incubated with an anti-PEX5 antibody and anti-mouse IgG peroxidase. As a loading control, anti-beta tubulin antibody is used. The blot is visualized with an enhanced chemiluminescent (ECL) kit. Western blot band intensity is measured by ImageJ. It is expected that adipocytes derived from one or more of the selected ASC populations will display significantly higher levels of PEX5 protein compared to the control.

PEX5 protein expression can also be measured using immunohistochemistry. Differentiated adipocytes are stained with DAPI and a fluorescence conjugated anti-PEX5 antibody and imaged using an epifluorescence microscope. Images are analyzed using ImageJ. The level of PEX5 expression is calculated as the average total fluorescence intensity per cell. It is expected that the adipocytes derived from one or more of the selected ASC populations will on average express PEX5 at significantly higher levels compared to the control.

In summary, this example shows, inter alia, that an ASC subtype can be identified and isolated that produces adipocytes expressing high amount of PEX5.

### Example 16: Engineering ASCs or CD34⁺ Cells to Secrete Gaussia Luciferase Upon Differentiation into Adipocytes

This example demonstrates, inter alia, the ability to genetically engineer ASCs or CD34⁺ cells to secrete gaussia luciferase (GLuc) upon differentiation into adipocytes.

In this example, ASCs and CD34⁺ cells are isolated and expanded as described in Examples 1 and 2. The cells are genetically labeled with two lentivirus reporter vectors expressing a green fluorescent protein (GFP) reporter gene (SEQ ID NO: 1) and a GLuc reporter gene (SEQ ID NO: 8). GFP expression is driven by the constitutive promoter CMV (pCMV) (SEQ ID NO: 3). GLuc expression is driven by the human adiponectin promoter (phAdipoQ) (SEQ ID NO: 4) in hASCs and hCD34⁺ cells or the murine adiponectin promoter (pmAdipoQ) (SEQ ID NO: 5) in mASCs. The adiponectin promoters drive adipocyte-specific expression of the GLuc reporter. The lentivirus vectors are used to genetically modify the ASCs and CD34⁺ cells following the method described in Example 9. The cells are then differentiated into adipocytes in vitro as described in Examples 3 and 4.

The expressions of the reporter proteins are characterized in the genetically modified cells as follows. GFP expression is assessed with flow cytometric analysis or fluorescence microscopy. Transduction efficiency is calculated as the percentage of GFP-expressing ASCs or CD34⁺ cells in total cells. The adipocytes derived from the transduced ASCs or CD34⁺ cells are also expected to express GFP. GLuc secretion is quantified using the Pierce™ Gaussia Luciferase Glow Assay kit (ThermoFisher, 16161) according to manufacturer's instructions. Briefly, the cell culture supernatant is collected and mixed with a buffer containing coelenterazine. The bioluminescence produced by GLuc results from the oxidation of coelenterazine, and the signal is measured using a luminometer. The adipocytes are expected to secrete a higher level of GLuc compared to the ASCs and CD34⁺ cells.

Overall, this example demonstrates, inter alia, the ability to generate and characterize adipocytes that secrete a reporter protein (GLuc) by engineering ASCs or CD34⁺ cells.

### Example 17A: Engineering ASCs or CD34⁺ Cells to Secrete Erythropoietin Upon Differentiation into Adipocytes

This example demonstrates, inter alia, the ability to genetically engineer ASCs or CD34⁺ cells to secrete erythropoietin (EPO) upon differentiation into adipocytes.

In this example, ASCs and CD34⁺ cells are isolated and expanded as described in Examples 1 and 2. The cells are genetically modified with a lentivirus vector expressing human EPO (SEQ ID NO: 9) or murine EPO (SEQ ID NO: 10). Human EPO expression is driven by the human adiponectin promoter (phAdipoQ) (SEQ ID NO: 4) in hASCs and hCD34⁺ cells, and the murine EPO expression is driven by the murine adiponectin promoter (pmAdipoQ) (SEQ ID NO: 5) in mASCs. The adiponectin promoters drive adipocyte-specific expression of EPO. The lentivirus vector is used to genetically modify the ASCs and CD34⁺ cells following the method described in Example 9. The cells are then differentiated into adipocytes in vitro as described in Examples 3 and 4.

EPO gene expression is quantified using quantified using reverse transcription-polymerase chain reaction (RT-PCR) following the procedure described in Example 3. The primer pairs for human EPO are 31 and 32 and for murine EPO are 33 and 34. GAPDH (human: primers 21 and 22; murine: primers 23 and 24) and actin (human: primers 25 and 26; murine: primers 27 and 28) are used as controls. It is expected that the level of EPO gene expression is higher in the adipocytes compared to the ASCs and CD34⁺ cells.

EPO secretion is measured using a standard enzyme-linked immunosorbent assay for human EPO (Abcam, ab119522) or murine EPO (Abcam, ab119593). Specifically, EPO specific antibodies have been precoated onto 96-well plates. The cell culture supernatants are collected and added to the wells along with a biotinylated EPO detection antibody. The microplate is then incubated at room temperature for 1 hour. Following washing with wash buffer, a Streptavidin-HRP conjugate is added to each well. The microplate is incubated at room temperature for 15 minutes, and unbound conjugates are then washed away using wash buffer. TMB is then added, and the microplate is incubated at room temperature for 10 minutes. The reaction is stopped by the addition of the Stop Solution, which changes the solution from blue to yellow. The density of yellow coloration is directly proportional to the amount of EPO captured in plate and is measured as absorbance on a spectrophotometer using 450 nm as the primary wavelength. It is expected that the genetically modified adipocytes secrete a higher level of EPO compared to the ASCs and CD34⁺ cells.

Overall, this example is expected to demonstrate, inter alia, the ability to generate and characterize adipocytes that secrete a mammalian serum protein, EPO, by engineering ASCs or CD34⁺ cells.

### Example 17B: Engineering ASCs Cells to Secrete Erythropoietin Upon Differentiation into Adipocytes (In Vitro)

This example demonstrates, inter alia, the ability to genetically engineer ASCs cells to secrete erythropoietin (EPO) in ASCs and upon differentiation into adipocytes.

In this example, hASCs were expanded as described in Example 5A and/or 5B. Once cells reached 70% confluence, they were passaged as described in Example 5A and/or 5B and seeded into 6 well culture plates at 1×10⁵ cells/well and allowed to culture overnight. The following day, cells were transfected with a pre-determined MOI, with a lentivirus reporter vector expressing a human EPO (hEPO) reporter gene (LV7) with a puromycin resistance gene. hEPO expression was driven by the human adiponectin promoter (phAdipoQ) in hASCs. hASCs were transfected as described in Example 16 the subsequently expanded as described in Example 5A and/or 5B then seeded for differentiation and differentiated as detailed in Example 7A and/or 7B. Media was then collected at day 6 and analyzed for hEPO presence using a hEPO ELISA kit. EPO secretion was measured using a standard enzyme-linked immunosorbent assay for human EPO (Biolegend, 442907). Specifically, EPO specific antibodies have been precoated onto 96-well plates. The cell culture supernatants were collected and diluted in assay buffer in pre-determined values then added to the wells. The plate was then incubated at room temperature for 2 hours on an orbital shaker. Following washing with wash buffer, a biotinylated EPO detection antibody was added to each well. The microplate was then incubated at room temperature for 2 hours on an orbital shaker. Following washing with wash buffer, a Streptavidin-HRP conjugate was added to each well. The microplate was incubated at room temperature for 30 minutes on an orbital shaker, and unbound conjugates were then washed away using wash buffer. Substrate solution F was then added, and the microplate was incubated at room temperature for 15 minutes. The reaction was stopped by the addition of the Stop Solution, which changes the solution from blue to yellow. The density of yellow coloration was directly proportional to the amount of EPO captured in plate and was measured as absorbance on a spectrophotometer using 450 nm as the primary wavelength and 560 nm as a background wavelength.

As shown in FIGS. 14A and 14B, hEPO was detected at ˜250 miU/ml in media wherein the hEPO engineered cells were growing while it was detected at very low background levels of ˜0.4 mIU/ml in media from unengineered control cells.

Overall, this example demonstrated, inter alia, the ability to generate and characterize adipocytes that secrete a mammalian serum protein, EPO specifically under an adipocyte specific promotor AdipoQ by engineering ASCs cells and then differentiating them.

### Example 17C: Engineering ASCs Cells to Secrete Gaussia Luciferase Upon Differentiation into Adipocytes (In Vitro)

This example demonstrates, inter alia, the ability to genetically engineer ASCs cells to secrete gaussia luciferase in ASCs and upon differentiation into adipocytes.

In this example, hASCs were expanded as described in Example 5A and/or 5B, and adipocytes were generated as described in Example 7A and/or 7B. The cells were genetically labeled with a lentivirus reporter expressing a GLuc reporter gene (LV1) under an adiponectin promoter as described in Example 16.

In this example, hASCs were expanded as described in Example 5A and/or 5B. Once cells reached 70% confluence, they were passaged as described in Example 5A and/or 5B and seeded into 6 well culture plates at 1×10⁵ cells/well and allowed to culture overnight. The following day, cells were transfected with a pre-determined MOI, with a lentivirus reporter vectors expressing a gaussia Luciferase reporter gene with a puromycin resistance gene. gLuc expression was driven by the human adiponectin promoter (phAdipoQ) in hASCs. Cells were then seeded for differentiation and differentiated as detailed in Example 7A and/or 7B. From day 3-day 7 of differentiation media was collected and analyzed for gaussia luciferase using the Pierce™ Gaussia Luciferase Glow Assay kit (ThermoFisher, 16161) according to manufacturer's instructions. Briefly, the media was collected and mixed with a buffer containing coelenterazine. The bioluminescence produced by GLuc results from the oxidation of coelenterazine, and the signal was measured using a luminometer. As shown in FIGS. 15A and 15B, ASCs secreted more GLuc as they were further differentiated into adipocytes.

Overall, this example demonstrates, inter alia, the ability to generate and characterize adipocytes that secrete gaussia Luciferase, by engineering ASCs cells.

### Example 18: Engineering ASCs or CD34⁺ Cells to Intracellularly Express Phenylalanine Hydroxylase Upon Differentiation into Adipocytes

This example demonstrates, inter alia, the ability to genetically engineer ASCs or CD34⁺ cells to express the intracellular enzyme phenylalanine hydroxylase (PAH) upon differentiation into adipocytes.

In this example, ASCs and CD34⁺ cells are isolated and expanded as described in Examples 1 and 2. The cells are genetically labeled with a lentivirus vector expressing human PAH (SEQ ID NO: 11) or murine PAH (SEQ ID NO: 12). Human PAH expression is driven by the human adiponectin promoter (phAdipoQ) (SEQ ID NO: 4) in hASCs and hCD34⁺ cells, and the murine PAH expression is driven by the murine adiponectin promoter (pmAdipoQ) (SEQ ID NO: 5) in mASCs. The adiponectin promoters drive adipocyte-specific expression of PAH. The lentivirus vector is used to genetically modify the ASCs and CD34⁺ cells following the method described in Example 9. The cells are then differentiated into adipocytes in vitro as described in Examples 3 and 4.

PAH gene expression in the genetically modified cells is quantified using reverse transcription-polymerase chain reaction (RT-PCR) following the procedure described in Example 3. The primer pairs for human PAH are 35 and 36 and for murine PAH are 37 and 38. GAPDH (human: primers 21 and 22; murine: primers 23 and 24) and actin (human: primers 25 and 26; murine: primers 27 and 28) are used as controls. It is expected that the level of PAH gene expression is higher in the adipocytes compared to the ASCs and CD34⁺ cells.

The PAH protein level in the engineered cells is measured using Western blot analysis. Total proteins from differentiated adipocytes in a 12-well plate are harvested by adding 200 μL of RIPA buffer onto each well. Next, 10 μg of cell lysate proteins are analyzed on 10-20% gradient polyacrylamide/SDS gel. After electrotransfer to a nitrocellulose membrane using dry transfer method, the blot is incubated with an anti-PAH antibody and anti-mouse IgG peroxidase. As a loading control, anti-beta tubulin antibody is used. The blot is visualized with an enhanced chemiluminescent (ECL) kit. Western blot band intensity is measured by ImageJ. It is expected that the engineered adipocytes will express a significantly higher level of PAH protein compared to the engineered ASCs and CD34⁺ cells.

PAH protein expression can also be measured using immunohistochemistry. Differentiated adipocytes are stained with DAPI and a fluorescence conjugated anti-PAH antibody and imaged using an epifluorescence microscope. Images are analyzed using ImageJ. The level of PAH expression is calculated as the average total fluorescence intensity per cell. It is expected that the engineered adipocytes will display a higher level of PAH fluorescence compared to the engineered ASCs and CD34⁺ cells.

Overall, this example is expected to demonstrate, inter alia, the ability to generate and characterize adipocytes that express an intracellular mammalian protein, PAH, by engineering ASCs or CD34⁺ cells.

### Example 19: In Vivo Secretion of Gaussia Luciferase by Adipocytes Derived from Transplanted Genetically Modified Adipogenic Cells

This example demonstrates, inter alia, the ability to achieve sustained in vivo secretion of gaussia luciferase by transplanting engineered adipogenic cells.

In this example, ASCs and CD34⁺ cells are isolated and expanded as described in Examples 1 and 2. The cells are genetically labeled with two lentivirus reporter vectors constitutively expressing a green fluorescent protein (GFP) reporter gene (SEQ ID NO: 1) and expressing a GLuc reporter gene (SEQ ID NO: 8) under an adiponectin promoter as described in Example 16. The cells are then differentiated into adipocytes in vitro as described in Examples 3 and 4.

The genetically modified ASCs, CD34⁺ cells, and differentiated adipocytes are transplanted into mice as described in Examples 5, 6, and 7, respectively. Secretion of GLuc is monitored via the serum level of GLuc. This level is quantified using the Pierce™ Gaussia Luciferase Glow Assay kit (ThermoFisher, 16161) according to manufacturer's instructions. In the transplanted mice, blood is drawn every seven days for up to six months post recovery. 5 μL blood is added to 1 μL of 20 mM EDTA and mixed with a buffer containing 100 μL of 100 μM coelenterazine. The bioluminescence produced by GLuc results from the oxidation of coelenterazine, and the signal is measured using a luminometer. It is expected that the serum level of GLuc in the transplanted mice will rise above the level in the control mice as early as the second week post recovery and will remain high up to six months.

Adipocyte engraftment from transplantation of the genetically modified ASCs, CD34⁺ cells, and differentiated adipocytes is assessed by harvesting the grafted tissues (in the case of ASCs and adipocytes only), the recipient mouse adipose depots (gonadal, perirenal, retroperitoneal, mesenteric, and inguinal), and non-adipose depot (lower hind limb skeletal muscle, liver, and lung) seven days post recovery and every month afterward up to six months. The harvested tissues are minced into ˜4 mm3 pieces and fixed in 1% paraformaldehyde for 15 min at room temperature. The fixed tissues are rehydrated in PBS 3×10 min each and stained with BODIPY-493/503 (ThermoFisher, D3922) (2 μg/ml to visualize the mature adipocytes), DAPI (ThermoFisher, D1306) (1 μg/ml, to visualize the nuclei), and anti-GFP antibody (to locate the transplanted cells) (Biolegend). The stained tissues are then washed and imaged using an epifluorescence microscope. Transplanted adipocytes and adipocytes derived from the transplanted ASCs or CD34⁺ cells stain positive for both GFP and BODIPY-493/503. Adipocyte engraftment is expected to be similar to the results observed in Examples 5, 6, and 7.

Overall, this example is expected to show, inter alia, that transplanting genetically modified adipogenic cells can lead to sustained secretion of GLuc protein in vivo.

### Example 20A: Therapeutic Effects in Mice by Transplanting Adipogenic Cells Genetically Modified to Produce Adipocytes Secreting EPO

This example demonstrates, inter alia, the ability to increase red blood cell production in vivo by transplanting adipogenic cells genetically modified to express EPO under an adiponectin promoter.

In this example, ASCs and CD34⁺ cells are isolated and expanded as described in Examples 1 and 2. The cells are genetically modified with a lentivirus vector expressing human EPO (SEQ ID NO: 9) under a human adiponectin promoter or murine EPO (SEQ ID NO: 10) under a murine adiponectin promoter as described in Example 17. The cells are then differentiated into adipocytes in vitro as described in Examples 3 and 4.

The genetically modified ASCs, CD34⁺ cells, and differentiated adipocytes are transplanted into mice as described in Examples 5, 6, and 7, respectively. Secretion of EPO is monitored via the serum levels of EPO, reticulocyte levels, and the hematocrit from whole blood. The procedures are described below.

In the transplanted mice, blood is drawn every seven days for up to six months post recovery. 18 μL of blood is mixed with 2 μL EDTA (0.2 mol/L) and placed into a 20 μL Drummond microcaps glass microcapillary tube (Sigma-Aldrich). After sealing one end of the tubes with Cha-seal (Chase Scientific Glass, Rockwood, TN), the capillary tubes are centrifuged in IEC MB Microhematocrit Centrifuge (DAMON/IEC Division, Needham, MA) for 3 minutes at 12,700×g. The capillary tubes are scanned (ScanMaker; Microtek, Santa Fe, CA), and digital images of the tubes are imported into Canvas X (ADB System, Seattle, WA). The packed cell volume ratio is determined.

After determining the hematocrit, the capillary tubes are snapped, and the plasma is collected for the measurement of plasma EPO levels. Plasma EPO level is quantified using a standard enzyme-linked immunosorbent assay for human EPO (Abcam, ab119522) or murine EPO (Abcam, ab119593) as described in Example 17.

To measure reticulocyte levels, 5 μL microliter of blood is mixed with 0.5 μL EDTA (0.2 mol/L) and analyzed using Retic-COUNT™, a thiazole orange reagent (BD Biosciences, 349204), as recommended by the manufacturer. Stained cells are analyzed on a flow cytometer, and the values are expressed as the percentage of reticulocytes relative to total erythrocytes.

It is expected that the hematocrit, plasma EPO levels, and reticulocyte levels in the transplanted mice will rise above the levels in the control mice as early as seven days post engraftment and will remain higher for up to six months.

Overall, this example is expected to show, inter alia, that transplanting adipogenic cells engineered to express EPO under an adiponectin promoter can lead to an increase in red blood cell production in mice.

### Example 20B: Therapeutic Effects in Mice by Transplanting ASCs and Adipogenic Cells Genetically Modified to Secrete EPO

This example demonstrates, inter alia, the ability to increase red blood cell production in vivo by transplanting ASCs and adipogenic cells derived from ASCs genetically modified to express EPO under an EF1a promoter.

In this example, hASCs were expanded as described in Example 5A and/or 5B. Once cells reached 70% confluence, they were passaged as described in Example 5A and/or 5B and seeded into 6 well culture plates at 1×10⁵ cells/well and allowed to culture overnight. The following day, cells were transfected with a pre-determined MOI, with a lentivirus reporter vectors expressing a human EPO (hEPO) reporter gene with a puromycin resistance gene. hASCs were subsequently expanded as described in Example 5A and/or 5B then seeded for differentiation and differentiated as detailed in Example 7A and/or 7B. Undifferentiated hASCs and differentiated hAdipocytes were transplanted into mice as described previously. In short NOD SCID mice (The Jackson Laboratory, 001303) were injected with ASCs or adipocytes derived from hASCs (hAdipocytes). The dorsal side of each mouse was swabbed with 70% ethanol, and the ASCs (16×10⁶ cells/side) and adipocytes (8×10⁶ cells/side) suspended in HBSS were injected using a 25G gauge syringe into the side of the dorsal flank for subcutaneous dosing. In the mock-transplanted cohort, an equal volume of HBSS alone or unengineered cells were injected. Post recovery, the mice were fed a high fat diet (Research Diets, D1245145% high fat diet product #NC9248609) for 28 days followed by normal chow diet (LabDiet, 5001) for the remainder of the study.

Mice were bled approximately weekly and blood was analyzed for the presence of hEPO protein and reticulocyte levels.

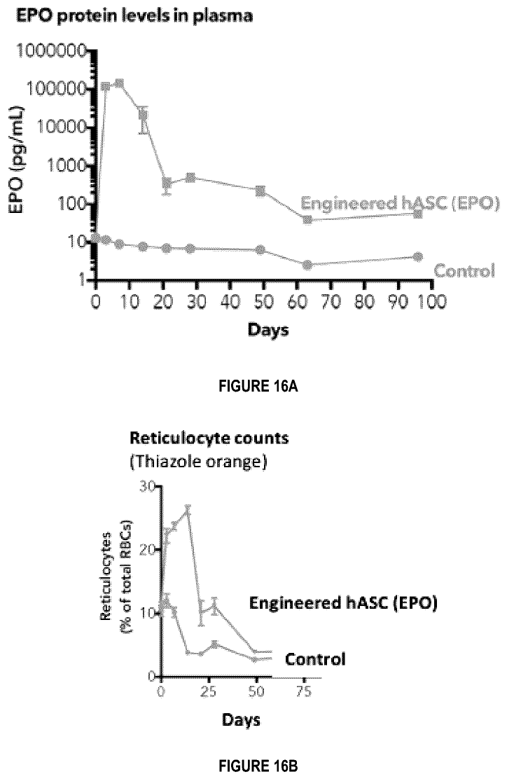

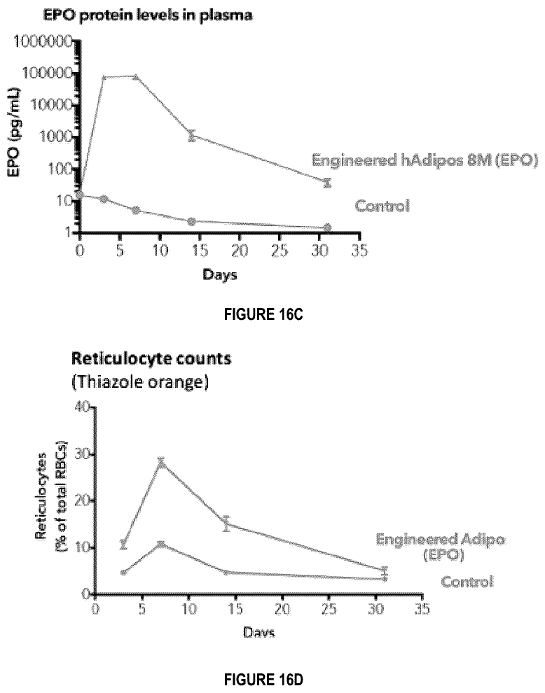

EPO secretion was measured using a qPCR-based immunoassay for human EPO (Thermo Fisher, A40419). Specifically, 5× diluted cell culture supernatant or mouse serum samples were combined with EPO specific oligo-conjugated antibodies and incubated at room temperature for 1 hour. A ligase and an additional splint oligo were added onto the plate. A qPCR protocol was run to generate a base DNA template which was then denatured and annealed for 40 cycles while measuring the fluorescence produced at each cycle. As shown in FIGS. 16A-16D, adipocytes and ASCs engineered to express hEPO secreted hEPO for the full duration of the study (100 days).

To measure reticulocyte levels, 5 μL microliter of blood was mixed with 1 mL of Retic-COUNT™, a thiazole orange reagent (BD Biosciences, 349204), as recommended by the manufacturer and 1 mL of PBS (control). Stained cells were analyzed on a flow cytometer using the Attune™ NxT No-Wash No-Lyse Filter Kit, and the values were expressed as the percentage of reticulocytes relative to total erythrocytes.

As shown in FIGS. 16A-16D, reticulocyte levels in the mice transplanted with hEPO expressing ASCs and Adipocytes rose above the levels in the control mice remained higher for 30+ days.

Overall, this example shows, inter alia, that transplanting ASCs and adipogenic cells engineered to express EPO can lead to an increase in red blood cell production in mice.

### Example 21: Therapeutic Effects of in PKU Mouse Model by Transplanting Adipogenic Cells Genetically Modified to Express PAH Upon Adipogenic Differentiation

This example demonstrates, inter alia, that transplanting adipogenic cells engineered to express PAH upon adipogenic differentiation leads to long-lasting reduction of hyperphenylalaninemia (HPA) in a PKU mouse model.

In this example, ASCs and CD34⁺ cells are isolated and expanded as described in Examples 1 and 2. The cells are genetically labeled with a lentivirus vector expressing human PAH (SEQ ID NO: 11) under a human adiponectin promoter or murine PAH (SEQ ID NO: 12) under a murine adiponectin promoter as described in Example 18. The cells are then differentiated into adipocytes in vitro as described in Examples 3 and 4.

PKU mice, which are homozygous Pah^{enu2-/-}, are generated by mating the heterozygous Pah^{enu2+/-} mice (B6.BTBR-Pah^{enu2}, The Jackson Laboratory, 029218). The genetically modified ASCs, CD34⁺ cells, and differentiated adipocytes are transplanted into four-week old PKU mice following procedures described in Examples 5, 6, and 7, respectively. The mice are maintained on a normal chow diet. Due to attenuated biosynthesis of melanin, hypopigmentation is one of the visible phenotypes of HPA. It is expected that this phenotype is significantly reversed in the transplanted mice. Specifically, as early as 2 weeks after engraftment, the transplanted mice are expected to show noticeably darker color than the control ones. The hair color in the transplanted mice will continue to darken overtime and may become undistinguishable from the wild-type mice after 2-4 months.

The effect of the transplantation on HPA is also measured by quantifying serum phenylalanine (Phe) concentration using a standard Phenylalanine Assay Kit (Millipore Sigma, MAK005). In the transplanted mice, serum is drawn every 7 days for up to 6 months post recovery. Serum is deproteinized before use in the assay with a 10 kDa MWCO spin filter. 10-50 μL of deproteinized serum is directly diluted to a final volume of 50 μL with the Phenylalanine Assay Buffer. The reaction is incubated for 20 minutes at 37° C., protected from light. Fluorescence intensity (λₑₓ=535 nm/λₑₘ=587 nm), which is proportional to the phenylalanine present, is measured using a fluorescence multiwell plate reader. It is expected that the serum Phe concentration in the transplanted mice is significantly reduced compared to the level in the control mice as early as 2 weeks post engraftment and remains low for the length of the study.

### Example 22: Non-Immunogenicity of ASCs in Culture

This example demonstrates, inter alia, that allogenic ASCs in culture have low immunogenicity.

In this example, mASCs were expanded as described in Example 5B and/or 19. Cells were plated at 2×10⁴ cells per well in 96 well plates. Murine lymphocytes were collected from primary mouse spleens via manual dissection followed by mechanical disruption with a 10 mL syringe plunger and homogenization by repeated pipetting. Solution filtered through a 70 μm cell strainer and washed with RPMI+10% FBS. Cells collected via centrifugation and red blood cell lysis using ammonium chloride. Spleens were collected from the following strains: C57, Balb/c, and FVB.

The immunogenicity of mASCs was characterized using a cytotoxicity assay. The responder cells in the cytotoxicity assay were mASCs derived from C57 mice. The effector cells in the cytotoxicity assay were splenocytes isolated from syngeneic (C57) and allogeneic (Balb/c and FVB) mice. YAC-1 was a murine lymphoma cell line that was used as a positive control for NK-mediated cytotoxicity.

The cytotoxicity assay was performed in 96-well microtiter plates. Target mASCs and YAC-1 cells were plated at 2×10⁴ cells per well. Effector cells (splenocytes) were added at various numbers ranging from 2×10⁵ to 2×10⁶ cells per well. C57 splenocytes serve as a syngeneic control, and Balb/c and FVB splenocytes serve as allogeneic effectors. Additional controls include mASCs alone, and YAC-1 cells alone. After 4 hours of incubation, CytoTox-Glo Assay Reagent (Promega) added to each well, incubated for 20 minutes and luminescence measured. Digitonin solution then added to wells to fully lyse all cells and luminescence measured after 20 minutes. Luminescence was directly correlated to the number of dead cells in each well.

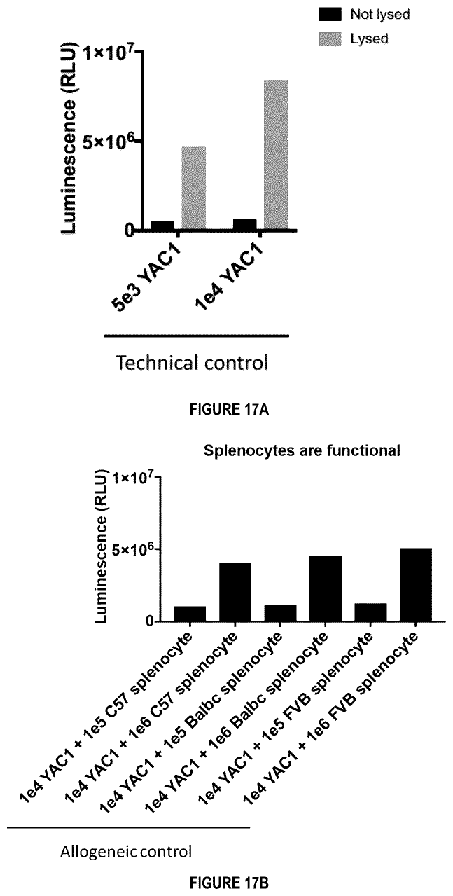

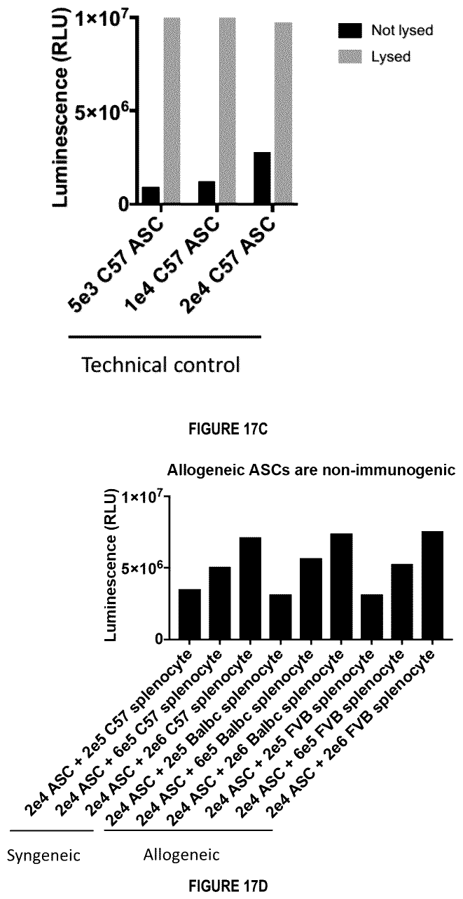

As shown in FIG. 17A, when YAC-1 cells were analyzed alone they have an RLU of ˜500k for 5k cells and an RLU of ˜640k before lysis, while after lysis the RLU goes up to ˜4.6M and ˜8.4M for the lysed cells thus demonstrating the positive technical control for the assay worked. Furthermore, when YAC-1 cells were co-cultured with splenocytes an increase in cell death can be detected as shown by a higher RLU when splenocytes are added to the YAC1 culture as shown in FIGS. 17A-17B, demonstrating that the splenocytes are functional. In FIG. 17C-17D, C57 ASCs were shown to display RLUs of ˜900k, 1.2M, 2.7M prelysis while post lysis they showed RLUs of 10-12M depending on how many cells were plated, again showing the positive technical control for the assay worked. When splenocytes were cocultured with mASCs very little cytotoxicity was observed as shown in FIG. 17B. Furthermore, cytotoxicity levels between syngeneic and allogeneic mASCs splenocyte co-cultures were similar. Although not wishing to be bound by any particular theory, these results demonstrate that allogeneic ASCs are non-immunogenic.

The immunogenicity was evaluated based on the change in cytoxicity when mASCs were co-cultured with allogeneic vs syngeneic splenocytes. It was shown that the all splenocytes were active toward YAC-1 while displaying very little cytotoxicity toward allogeneic and syngeneic mASCs. As shown in FIGS. 17A-17D, allogeneic splenocytes displayed cytotoxicity towards YAC-1 cells but not to mASCs.

In conclusion, the results in this example show, inter alia, that mASCs are non-immunogenic, as demonstrated by the lack of cell death in mixed lymphocyte assays. In summary, this example is expected to show, inter alia, that long-lasting reduction of HPA in a PKU mouse model can be achieved by transplanting adipogenic cells engineered to produce PAH-expressing adipocytes.

SEQUENCES

SEQ ID NO: 1 (GFP ORF; 720 bp):
ATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGCGAGGGCGATGCCACCTACGGCA
AGCTGACCCTGAAGTTCATCTGCACCACCGGCAAGCTGCCCGTGCCCTGGCCCACCCTCGTGA
CCACCCTGACCTACGGCGTGCAGTGCTTCAGCCGCTACCCCGACCACATGAAGCAGCACGACT
TCTTCAAGTCCGCCATGCCCGAAGGCTACGTCCAGGAGCGCACCATCTTCTTCAAGGACGACG
GCAACTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTAC
AACAGCCACAACGTCTATATCATGGCCGACAAGCAGAAGAACGGCATCAAGGTGAACTTCAAGA
TCCGCCACAACATCGAGGACGGCAGCGTGCAGCTCGCCGACCACTACCAGCAGAACACCCCC
ATCGGCGACGGCCCCGTGCTGCTGCCCGACAACCACTACCTGAGCACCCAGTCCGCCCTGAG
CAAAGACCCCAACGAGAAGCGCGATCACATGGTCCTGCTGGAGTTCGTGACCGCCGCCGGGA
TCACTCTCGGCATGGACGAGCTGTACAAGTAA
SEQ ID NO: 2 (Firefly luciferase (Luc) ORF; 1653 bp):
ATGGAAGATGCCAAAAACATTAAGAAGGGCCCAGCGCCATTCTACCCACTCGAAGACGGGACC
GCCGGCGAGCAGCTGCACAAAGCCATGAAGCGCTACGCCCTGGTGCCCGGCACCATCGCCTT
TACCGACGCACATATCGAGGTGGACATTACCTACGCCGAGTACTTCGAGATGAGCGTTCGGCT
GGCAGAAGCTATGAAGCGCTATGGGCTGAATACAAACCATCGGATCGTGGTGTGCAGCGAGAA
TAGCTTGCAGTTCTTCATGCCCGTGTTGGGTGCCCTGTTCATCGGTGTGGCTGTGGCCCCAGC
TAACGACATCTACAACGAGCGCGAGCTGCTGAACAGCATGGGCATCAGCCAGCCCACCGTCGT
ATTCGTGAGCAAGAAAGGGCTGCAAAAGATCCTCAACGTGCAAAAGAAGCTACCGATCATACAA
AAGATCATCATCATGGATAGCAAGACCGACTACCAGGGCTTCCAAAGCATGTACACCTTCGTGA
CTTCCCATTTGCCACCCGGCTTCAACGAGTACGACTTCGTGCCCGAGAGCTTCGACCGGGACA
AAACCATCGCCCTGATCATGAACAGTAGTGGCAGTACCGGATTGCCCAAGGGCGTAGCCCTAC
CGCACCGCACCGCTTGTGTCCGATTCAGTCATGCCCGCGACCCCATCTTCGGCAACCAGATCA
TCCCCGACACCGCTATCCTCAGCGTGGTGCCATTTCACCACGGCTTCGGCATGTTCACCACGCT
GGGCTACTTGATCTGCGGCTTTCGGGTCGTGCTCATGTACCGCTTCGAGGAGGAGCTATTCTT
GCGCAGCTTGCAAGACTATAAGATTCAATCTGCCCTGCTGGTGCCCACACTATTTAGCTTCTTC
GCTAAGAGCACTCTCATCGACAAGTACGACCTAAGCAACTTGCACGAGATCGCCAGCGGGGGG
GCGCCGCTCAGCAAGGAGGTAGGTGAGGCCGTGGCCAAACGCTTCCACCTACCAGGCATCCG
CCAGGGCTACGGCCTGACAGAAACAACCAGCGCCATTCTGATCACCCCCGAAGGGGACGACAA
GCCTGGCGCAGTAGGCAAGGTGGTGCCCTTCTTCGAGGCTAAGGTGGTGGACTTGGACACCG
GTAAGACACTGGGTGTGAACCAGCGCGGCGAGCTGTGCGTCCGTGGCCCCATGATCATGAGC
GGCTACGTTAACAACCCCGAGGCTACAAACGCTCTCATCGACAAGGACGGCTGGCTGCACAGC
GGCGACATCGCCTACTGGGACGAGGACGAGCACTTCTTCATCGTGGACCGGCTGAAGAGCCT
GATCAAATACAAGGGCTACCAGGTAGCCCCAGCCGAACTGGAGAGCATCCTGCTGCAACACCC
CAACATCTTCGACGCCGGGGTCGCCGGCCTGCCCGACGACGATGCCGGCGAGCTGCCCGCCG
CAGTCGTCGTGCTGGAACACGGTAAAACCATGACCGAGAAGGAGATCGTGGACTATGTGGCCA
GCCAGGTTACAACCGCCAAGAAGCTGCGCGGTGGTGTTGTGTTCGTGGACGAGGTGCCTAAAG
GACTGACCGGCAAGTTGGACGCCCGCAAGATCCGCGAGATTCTCATTAAGGCCAAGAAGGGCG
GCAAGATCGCCGTGTAA
SEQ ID NO: 3 (CMV promoter (pCMV); 589 bp):
TAGTTATTAATAGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTA
TTTACGGTAAACTGCCCACTTGGCAGTACATCAAGTGTATCATATGCCAAGTACGCCCCCTATTG
ACGTCAATGACGGTAAATGGCCCGCCTGGCATTATGCCCAGTACATGACCTTATGGGACTTTCC
TACTTGGCAGTACATCTACGTATTAGTCATCGCTATTACCATGGTGATGCGGTTTTGGCAGTACA
TCAATGGGCGTGGATAGCGGTTTGACTCACGGGGATTTCCAAGTCTCCACCCCATTGACGTCAA
TGGGAGTTTGTTTTGGCACCAAAATCAACGGGACTTTCCAAAATGTCGTAACAACTCCGCCCCA
TTGACGCAAATGGGCGGTAGGCGTGTACGGTGGGAGGTCTATATAAGCAGAGCTGGTTTAGTG
AACCGTCAGATC
SEQ ID NO: 4 (Human adiponectin promoter (phAdipoQ); 2741 bp):
CTCTTTCCACATGACGGCCTTTGTGGTGGGTGGCAGATTGCCCTGAGGCCTCGCAAAATGCTA
GGCTTTCACAATGTCACTGACTGACAGCCAGGCCCAGCACAGTCTTGGTGTGATTGTGGGGCT
AAAGTTATTCCACCTTGTGCAATAGCTACAGCTTTCTCTAACCAGCTGCATTCTTATAAAGTTAGA
AGAAAATACTTTTTTTTTTTTGAGATGGATTCTCGCTCTGTTGCCCAGGCTGGAGTGCAATGGTG
CGATCTCGGCTCGCTGCAACCTCCGCCTCCTGGGTTCAAACGATTCTCCTCCCTCAGACCCCC
GAGTAGCTGGGATTGCAGGTGCCTGCCACCACGCCCGGCTAACTTTTTTGTATTTTTAGTGGAG
ACGGGGTTTCACCATCTTCGTCAGGCTGGTCTCAGACTCCTGACCTCAAGTGATCTGCCCGCCT
CAGCCTCCCAAAATGCTGGGATTACAGGCATGAGCTACTGTGCCCGGCCAAAGAAAATACTTTT
TATGCCAGCCCTGAAACTACCCTGAAGCACATACATCAACCTTGAGGCCTCACACTCCATCAAG
AGGGGTGAAGGGCATGAGGAATTAGAAAGCATAGGGATTTTTAGTTAGACAGATCTGGTTCAAA
TCCTAGACTTGTGCCTTGAACAAATTATTTACCCTCATTGAACTCTAGATTCATTATTTGTAAAAT
GAAAGACAATAATAGTTATCTCCAAAGGAAAGTTGAATATGATCATTCATTTATTCATTAATTCAA
CATTTATTATTGCCTACTTTGTGCCAGGTTCTATTCTAGGAACTAAGGGATACAACTTTGAATAG
GCAAAATCTCTGCTCTCCTGAAGTTTACTTTTTTTTTTTTTTTTGAGACAGAGTTTCACTCTTGTCA
CCCAGGCTAGAGCGCAATGGTGCTCTTGGCTCACTGCAACCTCCACCTCCTGGGTTCAAGTGA
TTCTCTTGTCTCAGCCTCCCAAGTAGCTGGGACTACAGGTATGTGCCACCACGCCCGGCTATTT
CTGCATTTTTAGTAGAGATGGGGTTTCACCATGTTGGCCAGACTGGTCTCAAACTCCTGATCTCA
GGTGATATGCCTGTCTTGGCCTTCCAAAGTACTGGGATTACAGGCCTGAGCCACTGCACCTGAC
CTGAAGTTTATGTTCTATTAAATAGCAACAGACAGTAACATAAACCAAAAATAAATAGGAAAACAC
CATAACAAAAATCAAACAGTGATATAATTGAGAGTTGCTTCTATTTCTTTTTGTTGTCTTCTTGGTT
CAATCAGCCTGCTAAACTATATGGAACCTCATTTTCATGGGCCACTTATTTAAGCCGGGGGACC
TTGGAAAGTCTCTCATGTCTCTCATCTCAACGGCCTAATGTGACTTCTCTTGAAATATTTGGACAT
TAGCAGGAAGCTGAGGCTTTACATCAGATCTTTACTTTAATGGTGGACTTGACTTTACTGGTAGA
TTTTTAGGCTCTGTGTGGACTGTGGAGATGATATCTGGGGGGCAGGCAGACACTTGCCCTGCC
TCTGTCTGAGAAAATTCTGTTTTGGATGTCTTGTTGAAGTTGGTGCTGGCATCCTAAGCCCTTGC
TGGGGTCGTAATTTAATTCATCAGAATGTGTGGCTTGCAAGAACCGGCTCAGATCCTGCCCTTC
AAAAACAAAACATGAGCGTGCCAAGAAAGTCCAAGGTGTTGAATGTTGCCACTTCAAGCCTAAA
CTTTCTAGGAACACCTAAGTGGGTGGCAGCTTCCAGTTCTCCAGGCTGCTTCTAGGCCAGAGCT
GGGTTCCACAAGAGACAGAATAGGCATATATATGCTTAAGGAACTGGAAAAACAGGCTCTCTCT
CTCTCACAAACACACACACACACATACCAAGGTAGCTGTCAAAATGTTATCCGAAATTTTGGAAC
CAAAAAATCTTGAAAGATGGTATTCCAATATCACATTTTATGTAAGTTTTCTATTATATTAGATTCA
AATTACGATTCGAGGCCACAAGCTTTAAGAATTCAGGGCCTTTTTAACTTGCCAAGCCCCACACC
ACTCCAGGAACTTCCCCACACCCCAGTTCTCAGAATTCATGTGCAAGGTCTTTCCTAAATCCAG
GGTCCAGGTCAGAGAGTGGAGGATGTGCTCTATTTCTTACCTGATTGCAGACCCCTCTGACAGT
GCTCCCTTCTGAAGCACTCACTGTCTGAACGTACACAGTCTCAGACTTAATCATGCACAGTGAG
CAAGACTGTGGTGTGATAATTGGCGTCCCTGACTTATTAGGGCAAATCTATGGGAGGGGGAGA
CCTCCTGGACCACTGAGCAATTAATTCATTTACATTAGGAAGTTTCTCCGTCAGATGCAGGAAAA
AAATCTTGTTTTCCTGCTGTGGTTTTGACTTTTGCCCCATCTTCTGTTGCTGTTGTAGGAGGCAA
AATAAGGGTCAAGGCCTGGAAACACAAGTGCTTTGACTGAAGCTCCACTTGGCTTCCGAAGCCC
AAGCTGGGTTGTACCAGGTTCCCTAGGGTGCAGGCTGTGGGCAACTGCCAGGGACATGTGCCT
GCCCACCGGCCTCTGGCCCTCACTGAGTTGGCCAATGGGAAATGACAATTGTGAGGTGGGGAC
TGCCTGCCCCCGTGAGTACCAGGCTGTTGAGGCTGGGCCATCTCCTCCTCACTTCCATTCTGAC
TGCAGTCTGTGGTTCTGATTCCATACCAGAGGG
SEQ ID NO: 5 (Murine adiponectin promoter (pmAdipoQ); 2266 bp):
AAACCACCCAGCAAAAAACCAAACCGCCTAGCCTCAAGACATGTGTGGTTGAATGTTTTTCACTT
CTAGTCGCTAAGCAAGTGTGTGTTTTTACACAATGCCCTCTGTGGTGAGTGGCGGATTCCCCTG
AGAGTTCACCAAATGATAGGCTTTCACAATGCTCCCGGGTGTCTACCAGACCCAGCAAAGTATT
GATGTGGTTTTGGGGTGAAAGTCACTCTGTCTTGTGCAATAGTTAGAATCTGCTGAAACCAGCA
GTGTTCCTATATGGGACAGGGGTCCAGAGCTAACCCGGAGGCTATAACTGAGCAGAGGTGAAG
ACCACGAGGCATTGGGGAGCGTATGCCCTTTGTGGTCAGAGAGATCTAGCTTCGTGCCTTGGG
TCTGTGTCTCTCCCTCTTACTGGCTTCTGGCTTCTTCATTAAGTGGGAGACAACCACAGGTATCT
GTATGGGAAGACTCGACTACCCCTTGACTCAACATTGCTTGTTACTTACTTTGTACAAGATACTA
CTTAGTCTAGGGGTTATGGAGCATAACCTCAAGTAGGTAAAGCCCCTGCTCCAGCGTGTTTGCA
TTCCAGTAAGAAGCGAAAGACAGTAACACACATACAAAATAAGTAAGAAAATGCAACAACAGCAA
CAACAACAACACACACACACAAAGTAAGCAAAACGCTAAGGGAAAGATAGAGAGTGATACAGCT
TTGAGTTGCTGTAGTTCTTCTCTCTCCTTTGCTTCATACAGTTTGCTTGGGAAGTGTCCAGGGCC
ATGGGGTCACAACTAACAGCCCTTGGAAATGAGCTTGTGTCCTTAATCTTCATGACCTAACGTG
ATTTCTCTAGAAACATCAGTGCATTAACAGGAAGACAAGATGGAAGATCATATTTTGGCTCTCCT
TCCTTGGTGGGTTGACACTGCTGGTCCTATCCACTAGTAAAAGCATGACTCTTAGGCTCTGTGT
GGCCAGTGGAAGGTGGCAGTTGGAGGAAGCAGATGCTTGGCCAGCCTTTGCCTGGGAGCAGT
CTAGCTCTGAGTGTCTTATTGGAGCAGCTGCTGGCATCCAGAGTTCTTTTTGGATTCACGATTTA
ATTCAAAAGCTTTGTGCTCCCGAGAATCAGCTCTGGTCTTTCAAAAATAAGATGTGAGTCCGCC
GAGAGGCTCCCAAGGTATTGCCTTGCCAACTGCAAGCCTTTTAGGAGCAGTTTAGTGAGTGGTG
ACTGCTAGTTGCAGTTGGCTGTTAGCCCAGAGCTAATAATAGATAGAAAAGGTATATACTTAAGG
AGTCTGGAAACTGAGGTTTATCTACTCACAGAAAATGAGTTTCTAAAAAACTAGCTTGAAACTTA
CCCAGAAAAATCTTAGAACATGGTTCTCCAATGTCAAGGTAAGTGTTCTGTGACACTGGGCTTG
AATTATGTAGGGACCACAGATTTTAGAATTTGGACCCCTGAACTTGCTTCACACCCCACCAGGA
ACCTTCCTGTACAACAGCCCTCAGAATTCATCTACATGGTCTTTTCTCAGTATGGGATCCGGTCT
AGCAAGTGGAGCACACCTTCTATTGCTTAAAGATTTGTTTATGTATATGGGTATTTTGGCTGCAT
GCATATTTGCACACCAAAAGAAGGCAGCGGATCCCATGGAATTACTGTGGGTGCTGGGAATTGA
ACTCAGGACCTCTGGAAGAATAGCCAGTGCTCTTAACCACTGAGCCATGCCTGCAGTCCATCTA
TTTTTTATTCTAGTACAGCCCCTCTTCATTCTTACTGAAATAGTAATGCCTGAACCACACAGCTTC
ACATTTAGTTACAAAGAAAGAGTGGGAGTATCATGTGACAATTAGTGTTGTTGACTCTCCAGGAC
AAACTTATGGGAAAGGGAGGTCTCCTGACCCCTGAACAATCATTTTACTTGAGGATAATTTTCAT
TGCACTCAGAAACATGCTGAATTATTGTCCTTACCCTTGCCCCATCTCTTGCTCTGGTAGAGAAT
GGCCAAAGCCTGGAAACAGGATGGCTTGACAGAAGCTCTACTTGGCTTCCCAGACCCAAGCTG
GATTAAACCAGGTTCCCTAAGGAGTCTTAAGGCAGCTGCCAGGAGCAAGGGGCCCACTCATTG
GCTATTGGCCTTGACTGGGTTGGCCAATGGTAAGCTGGGGTCTGCCTGTCCCCATGAGTACCA
GACTAATGAGACCTGGCCACTTTCTCCTCATTTCTGTCTGTACGATTGTCAGTGGATCTGACGAC
ACCAAAAGGTAAGAAC
SEQ ID NO: 6 (Hygromycin B resistance ORF; 1026 bp):
ATGAAAAAGCCTGAACTCACCGCGACGTCTGTCGAGAAGTTTCTGATCGAAAAGTTCGACAGCG
TCTCCGACCTGATGCAGCTCTCGGAGGGCGAAGAATCTCGTGCTTTCAGCTTCGATGTAGGAG
GGCGTGGATATGTCCTGCGGGTAAATAGCTGCGCCGATGGTTTCTACAAAGATCGTTATGTTTA
TCGGCACTTTGCATCGGCCGCGCTCCCGATTCCGGAAGTGCTTGACATTGGGGAATTTAGCGA
GAGCCTGACCTATTGCATCTCCCGCCGTGCACAGGGTGTCACGTTGCAAGACCTGCCTGAAAC
CGAACTGCCCGCTGTTCTGCAGCCGGTCGCGGAGGCCATGGATGCGATCGCTGCGGCCGATC
TTAGCCAGACGAGCGGGTTCGGCCCATTCGGACCGCAAGGAATCGGTCAATACACTACATGGC
GTGATTTCATATGCGCGATTGCTGATCCCCATGTGTATCACTGGCAAACTGTGATGGACGACAC
CGTCAGTGCGTCCGTCGCGCAGGCTCTCGATGAGCTGATGCTTTGGGCCGAGGACTGCCCCG
AAGTCCGGCACCTCGTGCACGCGGATTTCGGCTCCAACAATGTCCTGACGGACAATGGCCGCA
TAACAGCGGTCATTGACTGGAGCGAGGCGATGTTCGGGGATTCCCAATACGAGGTCGCCAACA
TCTTCTTCTGGAGGCCGTGGTTGGCTTGTATGGAGCAGCAGACGCGCTACTTCGAGCGGAGGC
ATCCGGAGCTTGCAGGATCGCCGCGGCTCCGGGCGTATATGCTCCGCATTGGTCTTGACCAAC
TCTATCAGAGCTTGGTTGACGGCAATTTCGATGATGCAGCTTGGGCGCAGGGTCGATGCGACG
CAATCGTCCGATCCGGAGCCGGGACTGTCGGGCGTACACAAATCGCCCGCAGAAGCGCGGCC
GTCTGGACCGATGGCTGTGTAGAAGTACTCGCCGATAGTGGAAACCGACGCCCCAGCACTCGT
CCGAGGGCAAAGGAATAG
SEQ ID NO: 7 (Puromycin resistance ORF; 600 bp):
ATGACCGAGTACAAGCCCACGGTGCGCCTCGCCACCCGCGACGACGTCCCCAGGGCCGTACG
CACCCTCGCCGCCGCGTTCGCCGACTACCCCGCCACGCGCCACACCGTCGATCCGGACCGCC
ACATCGAGCGGGTCACCGAGCTGCAAGAACTCTTCCTCACGCGCGTCGGGCTCGACATCGGCA
AGGTGTGGGTCGCGGACGACGGCGCCGCGGTGGCGGTCTGGACCACGCCGGAGAGCGTCGA
AGCGGGGGGGGTGTTCGCCGAGATCGGCCCGCGCATGGCCGAGTTGAGCGGTTCCCGGCTG
GCCGCGCAGCAACAGATGGAAGGCCTCCTGGCGCCGCACCGGCCCAAGGAGCCCGCGTGGT
TCCTGGCCACCGTCGGCGTCTCGCCCGACCACCAGGGCAAGGGTCTGGGCAGCGCCGTCGTG
CTCCCCGGAGTGGAGGCGGCCGAGCGCGCCGGGGTGCCCGCCTTCCTGGAGACCTCCGCGC
CCCGCAACCTCCCCTTCTACGAGCGGCTCGGCTTCACCGTCACCGCCGACGTCGAGGTGCCC
GAAGGACCGCGCACCTGGTGCATGACCCGCAAGCCCGGTGCCTGA
SEQ ID NO: 8 (Gaussia luciferase (Gluc) ORF; 558 bp):
ATGGGAGTCAAAGTTCTGTTTGCCCTGATCTGCATCGCTGTGGCCGAGGCCAAGCCCACCGAG
AACAACGAAGACTTCAACATCGTGGCCGTGGCCAGCAACTTCGCGACCACGGATCTCGATGCT
GACCGCGGGAAGTTGCCCGGCAAGAAGCTGCCGCTGGAGGTGCTCAAAGAGATGGAAGCCAA
TGCCCGGAAAGCTGGCTGCACCAGGGGCTGTCTGATCTGCCTGTCCCACATCAAGTGCACGCC
CAAGATGAAGAAGTTCATCCCAGGACGCTGCCACACCTACGAAGGCGACAAAGAGTCCGCACA
GGGCGGCATAGGCGAGGCGATCGTCGACATTCCTGAGATTCCTGGGTTCAAGGACTTGGAGCC
CATGGAGCAGTTCATCGCACAGGTCGATCTGTGTGTGGACTGCACAACTGGCTGCCTCAAAGG
GCTTGCCAACGTGCAGTGTTCTGACCTGCTCAAGAAGTGGCTGCCGCAACGCTGTGCGACCTT
TGCCAGCAAGATCCAGGGCCAGGTGGACAAGATCAAGGGGGCCGGTGGTGACTAA
SEQ ID NO: 9 (Human erythropoietin (EPO) ORF; 582 bp):
ATGGGCGTGCACGAGTGCCCCGCCTGGCTGTGGCTGCTGCTGAGCCTGCTGAGCCTGCCCCT
GGGCCTGCCCGTGCTGGGCGCCCCCCCCCGGCTGATCTGCGACAGCCGGGTGCTGGAGCGG
TACCTGCTGGAGGCCAAGGAGGCCGAGAACATCACCACCGGCTGCGCCGAGCACTGCAGCCT
GAACGAGAACATCACCGTGCCCGACACCAAGGTGAACTTCTACGCCTGGAAGCGGATGGAGGT
GGGCCAGCAGGCCGTGGAGGTGTGGCAGGGCCTGGCCCTGCTGAGCGAGGCCGTGCTGCGG
GGCCAGGCCCTGCTGGTGAACAGCAGCCAGCCCTGGGAGCCCCTGCAGCTGCACGTGGACAA
GGCCGTGAGCGGCCTGCGGAGCCTGACCACCCTGCTGCGGGCCCTGGGCGCCCAGAAGGAG
GCCATCAGCCCCCCCGACGCCGCCAGCGCCGCCCCCCTGCGGACCATCACCGCCGACACCTT
CCGGAAGCTGTTCCGGGTGTACAGCAACTTCCTGCGGGGCAAGCTGAAGCTGTACACCGGCG
AGGCCTGCCGGACCGGCGACCGGTGA
SEQ ID NO: 10 (Murine EPO ORF; 576 bp):
ATGGGGGTGCCCGAACGTCCCACCCTGCTGCTTTTACTCTCCTTGCTACTGATTCCTCTGGGCC
TCCCAGTCCTCTGTGCTCCCCCACGCCTCATCTGCGACAGTCGAGTTCTGGAGAGGTACATCTT
AGAGGCCAAGGAGGCAGAAAATGTCACGATGGGTTGTGCAGAAGGTCCCAGACTGAGTGAAAA
TATTACAGTCCCAGATACCAAAGTCAACTTCTATGCTTGGAAAAGAATGGAGGTGGAAGAACAG
GCCATAGAAGTTTGGCAAGGCCTGTCCCTGCTCTCAGAAGCCATCCTGCAGGCCCAGGCCCTG
CTAGCCAATTCCTCCCAGCCACCAGAGACCCTTCAGCTTCATATAGACAAAGCCATCAGTGGTC
TACGTAGCCTCACTTCACTGCTCCGGGTGCTGGGAGCTCAGGAATTGATGTCACCTCCAGATAC
CACCCCACCTGCTCCACTCCGAACACTCACAGTGGATACTTTCTGCAAGCTCTTCCGGGTCTAC
GCCAACTTCCTCCGGGGGAAACTGAAGCTGTACACGGGAGAGGTCTGCAGGAGAGGGGACAG
GTGA
SEQ ID NO: 11 (Human phenylalanine hydroxylase (PAH) ORF; 1359 bp):
ATGTCCACTGCGGTCCTGGAAAACCCAGGCTTGGGCAGGAAACTCTCTGACTTTGGACAGGAA
ACAAGCTATATTGAAGACAACTGCAATCAAAATGGTGCCATATCACTGATCTTCTCACTCAAAGA
AGAAGTTGGTGCATTGGCCAAAGTATTGCGCTTATTTGAGGAGAATGATGTAAACCTGACCCAC
ATTGAATCTAGACCTTCTCGTTTAAAGAAAGATGAGTATGAATTTTTCACCCATTTGGATAAACGT
AGCCTGCCTGCTCTGACAAACATCATCAAGATCTTGAGGCATGACATTGGTGCCACTGTCCATG
AGCTTTCACGAGATAAGAAGAAAGACACAGTGCCCTGGTTCCCAAGAACCATTCAAGAGCTGGA
CAGATTTGCCAATCAGATTCTCAGCTATGGAGCGGAACTGGATGCTGACCACCCTGGTTTTAAA
GATCCTGTGTACCGTGCAAGACGGAAGCAGTTTGCTGACATTGCCTACAACTACCGCCATGGG
CAGCCCATCCCTCGAGTGGAATACATGGAGGAAGAAAAGAAAACATGGGGCACAGTGTTCAAG
ACTCTGAAGTCCTTGTATAAAACCCATGCTTGCTATGAGTACAATCACATTTTTCCACTTCTTGAA
AAGTACTGTGGCTTCCATGAAGATAACATTCCCCAGCTGGAAGACGTTTCTCAATTCCTGCAGA
CTTGCACTGGTTTCCGCCTCCGACCTGTGGCTGGCCTGCTTTCCTCTCGGGATTTCTTGGGTGG
CCTGGCCTTCCGAGTCTTCCACTGCACACAGTACATCAGACATGGATCCAAGCCCATGTATACC
CCCGAACCTGACATCTGCCATGAGCTGTTGGGACATGTGCCCTTGTTTTCAGATCGCAGCTTTG
CCCAGTTTTCCCAGGAAATTGGCCTTGCCTCTCTGGGTGCACCTGATGAATACATTGAAAAGCT
CGCCACAATTTACTGGTTTACTGTGGAGTTTGGGCTCTGCAAACAAGGAGACTCCATAAAGGCA
TATGGTGCTGGGCTCCTGTCATCCTTTGGTGAATTACAGTACTGCTTATCAGAGAAGCCAAAGC
TTCTCCCCCTGGAGCTGGAGAAGACAGCCATCCAAAATTACACTGTCACGGAGTTCCAGCCCCT
GTATTACGTGGCAGAGAGTTTTAATGATGCCAAGGAGAAAGTAAGGAACTTTGCTGCCACAATA
CCTCGGCCCTTCTCAGTTCGCTACGACCCATACACCCAAAGGATTGAGGTCTTGGACAATACCC
AGCAGCTTAAGATTTTGGCTGATTCCATTAACAGTGAAATTGGAATCCTTTGCAGTGCCCTCCAG
AAAATAAAGTAA
SEQ ID NO: 12 (Murine phenylalanine hydroxylase (PAH) ORF; 1362 bp):
ATGGCAGCTGTTGTCCTGGAGAACGGAGTCCTGAGCAGAAAACTCTCAGACTTTGGGCAGGAA
ACAAGTTACATCGAAGACAACTCCAATCAAAATGGTGCTGTATCTCTGATATTCTCACTCAAAGA
GGAAGTTGGTGCCCTGGCCAAGGTCCTGCGCTTATTTGAGGAGAATGAGATCAACCTGACACA
CATTGAATCCAGACCTTCTCGTTTAAACAAAGATGAGTATGAGTTTTTCACCTATCTGGATAAGC
GTAGCAAGCCCGTCCTGGGCAGCATCATCAAGAGCCTGAGGAACGACATTGGTGCCACTGTCC
ATGAGCTTTCCCGAGACAAGGAAAAGAACACAGTGCCCTGGTTCCCAAGGACCATTCAGGAGC
TGGACAGATTCGCCAATCAGATTCTCAGCTATGGAGCCGAACTGGATGCAGACCACCCAGGCT
TTAAAGATCCTGTGTACCGGGCGAGACGAAAGCAGTTTGCTGACATTGCCTACAACTACCGCCA
TGGGCAGCCCATTCCTCGGGTGGAATACACAGAGGAGGAGAGGAAGACCTGGGGAACGGTGT
TCAGGACTCTGAAGGCCTTGTATAAAACACATGCCTGCTACGAGCACAACCACATCTTCCCTCTT
CTGGAAAAGTACTGCGGTTTCCGTGAAGACAACATCCCGCAGCTGGAAGATGTTTCTCAGTTTC
TGCAGACTTGTACTGGTTTCCGCCTCCGTCCTGTTGCTGGCTTACTGTCGTCTCGAGATTTCTTG
GGTGGCCTGGCCTTCCGAGTCTTCCACTGCACACAGTACATTAGGCATGGATCTAAGCCCATGT
ACACACCTGAACCTGATATCTGTCATGAACTCTTGGGACATGTGCCCTTGTTTTCAGATAGAAGC
TTTGCCCAGTTTTCTCAGGAAATTGGGCTTGCATCGCTGGGGGCACCTGATGAGTACATTGAGA
AACTGGCCACAATTTACTGGTTTACTGTGGAGTTTGGGCTTTGCAAGGAAGGAGATTCTATAAA
GGCATATGGTGCTGGGCTCTTGTCATCCTTTGGAGAATTACAGTACTGTTTATCAGACAAGCCA
AAGCTCCTGCCCCTGGAGCTAGAGAAGACAGCCTGCCAGGAGTATACTGTCACAGAGTTCCAG
CCCCTGTACTATGTGGCCGAGAGTTTCAATGATGCCAAGGAGAAAGTGAGGACTTTTGCTGCCA
CAATCCCCCGGCCCTTCTCCGTTCGCTATGACCCCTACACTCAAAGGGTTGAGGTCCTGGACAA
TACTCAGCAGTTGAAGATTTTAGCTGACTCCATTAATAGTGAGGTTGGAATCCTTTGCCATGCCC
TGCAGAAAATAAAGTCATGA
SEQ ID NO: 13 (Murine aP2/Fabp4 promoter; 857 bp):
GGATGAACTGCTCCGCCCTCTGTCTCTTTGGCAGGGTTGGAGCCCACTGTGGCCTGAGCGACT
TCTATGGCTCCCTTTCTGTGATTTTCATGGTTTCTGAGCTCTTTTCCCCCGCTTTATGATTTTCTC
TTTTTGTCTCTCTCTTGCTAAACCTCCTTCGTATATGCCCTCTCAGGTTTCATTTCTGAATCATCT
ACTGTGAACTATTCCCATTGTTTGCCAGAAGCCCCCTGGTTCTTCCTTCTAGAAGGAATAATGGG
GGGAAGTTCAATGCATTAGCTTTTGACAGTCAAAACAGGAACCTTTAAAATACTCTGTTCATGGT
TAAAAATAATTTGTACTCTAAGTCCAGTGATCATTGCCAGGGAGAACCAAAGTTGAGAAATTTCT
ATTAAAAACATGACTCAGAGGAAAACATACAGGGTCTGGTCATGAAGGAAATGATCTGGCCCCC
ATTGGTCACTCCTACAGTCACATGGTCAGGGCATCTTTAAAAGTGAGCTATCTGGACTTCAGAG
GCTCATAGCACCCTCCTGTGCTGCAGCCTTTCTCACCTGGAAGACAGCTCCTCCTCGAAGGTTT
ACAAA

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

As used herein, all headings are simply for organization and are not intended to limit the disclosure in any manner. The content of any individual section may be equally applicable to all sections.

### INCORPORATION BY REFERENCE

All patents and publications referenced herein are hereby incorporated by reference in their entireties.

1. An allogenic, non-immunogenic, long-acting composition comprising a therapeutically effective amount of substantially pure adipogenic cells.

2-4. (canceled)

5. The composition of claim 1, wherein the composition does not substantially result in an inflammatory reaction upon administration.

6. The composition of claim 1, wherein the composition elicits less than about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% increase in TNF-alpha, IL-2, or IFN-gamma, or any combination thereof, upon administration to a subject.

7. The composition of claim 1, wherein the composition elicits an increase of about 5%, about 10%, about 15%, about 20%, about 25%, about 30%, about 35%, about 40%, about 45%, about 50%, about 60%, about 70%, about 80%, about 90%, about 100%, about 150%, about 200%, about 250%, about 300%, about 350%, or about 400% or more of IDO, HLA-G, HGF, PGE2, TGFbeta, and IL-6, or any combination thereof, upon administration to a subject.

8. The composition of claim 1, wherein the adipogenic cells are selected from adipocytes, adipogenic stem cells (ASCs), and CD34⁺ cells.

9. (canceled)

10. (canceled)

11. The composition of claim 8, wherein the adipocytes express and/or secrete one or more of CIDEC, FABP4, PLIN1, LGALS12, ADIPOQ, TUSC5, SLC19A3, PPARG, LEP, CEBPA, or a combination thereof.

12. The composition of claim 8, wherein the adipocytes are characterized as having one or more, 2 or more, 3 or more, 4 or more, 5 or more, 10 or more, 15 or more, 20 or more, 25 or more, 30 or more, or 35 or more of the following:
a. being post-mitotic;
b. having a lipid content of greater than about 35% (% fresh weight of adipose tissue); optionally greater than about 40%, about 45%, about 50%, about 55%, about 60%, about 65%, about 70%, about 75%, or about 80%;
c. having a fat content in adipose tissue of about 60% to about 95%, optionally 60-94%, about 60% to about 90%, about 60% to about 85%, about 60% to about 80%, about 60% to about 75%, about 60% to about 70%, about 60% to about 65%, about 65% to about 90%, about 70% to about 90%, about 75% to about 90%, about 80% to about 90%, or about 85% to about 90%;
d. having an average fat content of about 80%, optionally about 75 to about 85%;
e. having a water content in adipose tissue of about 5% to about 40%, optionally about 6-36%, about 5% to about 35%, about 5% to about 30%, about 5% to about 25%, about 5% to about 20%, about 5% to about 15%, about 5% to about 10%, about 10% to about 40%, about 15% to about 40%, about 20% to about 40%, about 25% to about 40%, about 30% to about 40%, or about 35% to about 40%);
f. having an average water content of about 15%, optionally about 12.5% to about 17.5%;
g. having a specific gravity of about 1 g/mL, optionally 0.916 g/mL, about 0.5 g/mL, about 0.6 g/mL, about 0.7 g/mL, about 0.8 g/mL, about 0.9 g/mL, about 1.1 g/mL, or about 1.2 g/mL;
h. having a lipid content comprising one or more of stearic acid, oleic acid, linoleic acid, palmitic acid, palmitoleic acid, and myristic acid, a derivative thereof;
i. having a lipid content comprising one or more of free fatty acids, cholesterol, monoglycerides, and diglycerides;
j. having a lipid droplet of a size greater than about 90% of the cell volume, optionally greater than 95% or greater than about 98%, or about 93%, or about 95%, or about 97%, or about 99%;
k. having a lipid droplet comprising at least about 30% to about 99% of the volume of the cell; optionally at least about 40% to about 90%, about 50% to about 90%, about 60% to about 90%, about 70% to about 90% about 80% to about 90%, about 50%, about 60%, about 70%, about 80%, or about 90%;
l. having a surface size of about 20-300 μm in diameter, optionally about 20-300 μm, about 20-200 μm, about 20-100 μm, about 20-500 μm, about 20-30 μm, about 50-300 μm, about 50-200 μm, about 50-100 μm, about 100-300 μm, about 100-200 μm, about 150-300 μm, about 150-200 μm, or about 200-300 μm;
m. having a nucleus volume of about 200-400 μm³, optionally about 200 to about 350 μm³, about 200 to about 300 μm³, about 200 to about 250 μm³, about 250 to about 400 μm³, about 250 to about 350 μm³, about 250 to about 300 μm³, about 300 to about 350 μm³ or about 300 to about 400 μm³;
n. having a total volume of about 4,000-18,000 μm³, optionally about 4000 to about 15000 μm³, about 5000 to about 15000 μm³, about 10000 to about 15000 μm³, about 12500 to about 15000 μm³, about 4000 to about 10000 μm³, about 5000 to about 15000 μm³, about 7500 to about 15000 μm³, about 10000 to about 15000 μm³, about 12500 to about 15000 μm³;
o. having a nucleus to cell ratio of about 1:20-1:90, optionally about 1:20 to about 1:80, about 1:20 to about 1:70, about 1:20 to about 1:60, about 1:20 to about 1:50, about 1:20 to about 1:40, about 1:20 to about 1:30; about 1:30 to about 1:80, about 1:40 to about 1:80, about 1:50 to about 1:80, about 1:60 to about 1:80, or about 1:70 to about 1:80;
p. having a flattened nucleus;
q. having a small cytoplasm of less than about 10% to about 60% of total cell volume, wherein the cytoplasm excludes lipid droplets volume, optionally less than about 20%, less than about 30%, less than about 40%, or less than about 50%;
r. being capable of absorbing and releasing liquids;
s. being buoyant in in water or an aqueous solution, optionally media, or HBSS;
t. having a non-centrally located nucleus;
u. having one or more fat droplets;
v. having a non-spherical cytoplasm;
w. being capable of secreting one or more of adiponectin, leptin, and TNF-alpha;
x. being capable of lipogenesis;
y. being capable of storing triglycerides (TG);
z. being capable of secreting non-esterified fatty acids (NEFA), optionally long chain fatty acids such as oleic acid palmitoleic acid, linoleic acid, arachidonic acid, lauric acid, and stearic acid;
aa. being responsive to hormones;
bb. being responsive to neural input;
cc. having a cell turn-over rate of about 9 years, optionally about 8 to about 10 years;
dd. having an average diameter of about 45 μm, optionally about 47.2 μm, about 40 μm, about; 42.5 μm, about 47.5 μm, or about 50 μm;
ee. a cell population having a diameter distribution wherein about 25% of cells have a diameter of less than about 50 μm; about 40% of cells have a diameter of about 50-69 μm; about 25% of cells have a diameter of about 70-89 μm, and about 10% of cells have a diameter of greater than or equal to about 90 μm;
ff. responsive to atrial natriuretic peptide (ANP);
gg. capable of lipolysis;
hh. expressing receptors that can bind and respond to steroid hormones;
ii. lysed due to phosphatidylcholine;
jj. cell density of about 1 g/ml, optionally about 0.8 g/ml, about 0.9 g/ml, about 1.1 g/ml, about 1.2 g/ml;
kk. greater than about 80% viability, optionally about 85%, about 90%, about 95%, about 97%, about 98%, or about 99%;
ll. greater than about 80% purity, optionally about 85%, about 90%, about 95%, about 97%, about 98%, or about 99%,
mm. adequate potency, optionally amount of Oil Red O eluted greater than about 200 μg/ml; and
nn. negative for microbial contamination.

13-18. (canceled)

19. The composition of claim 8, wherein the ASCs are characterized as having one or more, or one, two, three of the following:
a. viability of about 90% or greater;
b. glucose uptake of about 5 mmol/L to about 10 mmol/L;
c. and lactate production of about 10 mmol/L to about 15 mmol/L.

20. The composition of claim 8, wherein the ASCs express elevated levels of one or more of CDw210, CD107b, CD164, CD253, CD361, CD120b, CD213A1 CDw210b, CD340 and CDw293 or any combination thereof compared to wild type ASCs and/or unenriched ASCs.

21. The composition of claim 8, wherein the ASCs express reduced levels of one or more of CD266, CD151, CD49c, CD9, CD167, CD325, CD115 CD10, CD26, and CD142 or any combination thereof compared to wild type ASCs and/or unenriched ASCs.

22.-25. (canceled)

26. The composition of claim 8, wherein less than about 5% of ASCs express one or more of the surface markers HLAII, CD11b, CD11c, CD14, CD45, CD31, CD34, CD80 and CD86.

27. The composition of claim 8, wherein at least about 90% or at least about 95% of the ASCs express one or more of the surface markers HLA I, CD29, CD44, CD59, CD73, CD90, and CD105.

28.-35. (canceled)

36. The composition of claim 1, wherein the adipogenic cells, upon administration to a subject, provide a therapeutically effective amount of one or more of erythropoietin (EPO); adipsin; phenylalanine hydroxylase (PAH); adiponectin; PEX5; ATP:cob(1)alamin adenosyl transferase (MMAB); 14-3-3 protein epsilon; 2-oxoisovalerate dehydrogenase subunit alpha, mitochondrial, BCKDHA; 2-Oxoisovalerate dehydrogenase subunit beta, mitochondrial, BCKDHB; 3-Hydroxyisobutyrate dehydrogenase (HIBADH); 3-Hydroxyisobutyryl-CoA deacylase (HIBCH); 3-Methylcrotonyl CoA carboxylase, MCCC1; 3-Methylcrotonyl CoA carboxylase, MCCC2; 4-Aminobutyrate-α-ketoglutarate aminotransferase (ABAT); 5-nucleotidase; 6-phosphogluconate dehydrogenase, decarboxylating; medium-chain acyl-CoA dehydrogenase, MCAD; short-chain acyl-CoA dehydrogenase, SCAD; very long-chain acyl-CoA dehydrogenase, VLCAD; Acetyl-CoA thiolase (acetyl-coenzyme A acetyltransferase), ACAT1; Acid ceramidase; Adenine phosphoribosyltransferase, APRT; Adenosine deaminase; Adipocyte enhancer-binding protein 1; Agrin; Aldehyde oxidase; Aldo-keto reductase family 1 member C2; Alkaline phosphatase, tissue-nonspecific isozyme; Alkyldihydroxyacetonephosphate synthase, AGPS; Alpha-2-macroglobulin; Alpha-enolase; Alpha-fetoprotein; Alpha-L-iduronidase, Alpha-N-acetylglucosaminidase; Alpha-N-acetylglucosaminidase 82 kDa form; Alpha-N-acetylglucosaminidase 77 kDa form; Aminoacylase-1; Angiotensinogen; Angiotensin-1; Angiotensin-2; Angiotensin-3; Angiotensin-4; Angiotensin 1-9; Angiotensin 1-7; Angiotensin 1-5; Angiotensin 1-4; Annexin A5; Adaptor Related Protein Complex 3 Subunit Beta 1, AP3B1; Apolipoprotein E; Argininosuccinate lyase, ASL; Argininosuccinate synthase; Argininosuccinic acid synthetase, ASS; Arylsulfatase A; Arylsulfatase A component B; Arylsulfatase A component C; Arylsulfatase B; aspartylglucosaminidase; ATP-binding cassette transporter, ABCD1; ATP-dependent RNA helicase, DDX3X; Endorepellin; Beta-2-microglobulin; Beta-galactosidase; Beta-hexosaminidase subunit alpha, HEXA; Beta-hexosaminidase subunit beta, HEXB; Bifunctional purine biosynthesis protein, PURH; Biglycan; Biotinidase; Biotinidase; Bone morphogenetic protein 1; Branching enzyme, GBE1; Calmodulin; Calreticulin; cAMP-dependent protein kinase catalytic subunit gamma; Cartilage oligomeric matrix protein; Cartilage-associated protein; Catalase; Catalase, CAT; Cathepsin A; Cathepsin B; Cathepsin D; Cathepsin F; Cathepsin K; Citrin, SLC25A13; Collagen alpha-1(1) chain; Collagen alpha-1(III) chain; Collagen alpha-1(IV) chain; Arresten; Collagen alpha-1(V) chain, Collagen alpha-1(XI) chain, Collagen alpha-1(XVIII) chain; Endostatin, Collagen alpha-2(I) chain; Collagen alpha-2(IV) chain; Canstatin; Collagen alpha-2(V) chain; Collagen alpha-2(VI) chain; Collagen alpha-3(VI) chain; Complement C1r subcomponent; Complement C1s subcomponent; Complement C3; Complement C4 beta chain; Complement factor D; Carnitine palmitoyltransferase 1A, CPT1A; Cystathionine β-synthase, CBS; Cystatin-C; Cystinosin, CTNS; Cytochrome c; Cytokine receptor-like factor 1; Cytoplasmic acetoacetyl-CoA thiolase, ACAT2; D-bifuncitonal enzyme, HSD17B4; Decorin; Dihydrolipoyl dehydrogenase, mitochondrial; Dihydroxyacetonephosphate acyltransferase, GNPAT; Dipeptidyl peptidase 1; Cathepsin C; EGF-containing fibulin-like extracellular matrix protein 1; EGF-containing fibulin-like extracellular matrix protein 2; Elastin; Elongation factor 2; Electron Transfer Flavoprotein Subunit Alpha, ETFA; Electron Transfer Flavoprotein Subunit Beta, ETFB; Electron transfer flavoprotein dehydrogenase, ETFDH; Extracellular matrix protein 1; Fibrillin-1; Fibrillin-2; Fibronectin; Fibulin-1; Fibulin-5; Formyl-Glycin generating enzyme, SUMF1; Fructose 1,6-biphosphatase, FBP1; Fumarylacetoacetase; Fumarylacetoacetate hydrolase domain-containing protein 2A, FAHD2A; Galactocerebrosidase; Galactokinase 1; Galactose-1-phosphate uridyl transferase, GALT; Ganglioside GM2 activator; Ganglioside GM2 activator isoform short; Gelsolin; GIcNAc phosphotransferase, GNPTA; Glucose-6-phosphate 1-dehydrogenase; Glucose-6-phosphate isomerase; Glucose-6-phosphate translocase, G6PT1; Glutaryl CoA dehydrogenase, GCDH; Glutathione peroxidase 3; Glutathione synthetase; Glycerol kinase; Glycerol-3-phosphate dehydrogenase [NAD(+)], cytoplasmic; Glycine cleavage enzyme system, AMT; Glycine cleavage enzyme system, GCSH; Glycogen debranching enzyme; 4-alpha-glucanotransferase; Amylo-alpha-1,6-glucosidase; Glycogen phosphorylase, liver form; Glypican-1; Glypican-6; Hydroxyacyl-CoA Dehydrogenase Trifunctional Multienzyme Complex Subunit Alpha, HADHA; Haptoglobin; Heparan N-sulfatase, N-sulfoglucosamine sulfohydrolase, SGSH; Heparan-alpha-glucosaminide N-acetyltransferase, HGSNAT; Hormone-sensitive lipase; Hydroxyacyl-coenzyme A dehydrogenase, mitochondrial; Hyperactivity of glutamate dehydrogenase, GLUD1; Hypoxanthine-guanine phosphoribosyltransferase, HPRT; Iduronate-2-sulfatase, IDS; Insulin-like growth factor-binding protein 7; Interstitial collagenase; Isovaleryl-CoA dehydrogenase; Keratin, type II cytoskeletal 1; Keratin, type II cytoskeletal 6B; L-lactate dehydrogenase A chain; L-lactate dehydrogenase B chain; Lactoylglutathione lyase; Laminin subunit alpha-2; Laminin subunit alpha-4; Laminin subunit beta-1; Laminin subunit beta-2; Laminin subunit gamma-1; Leptin; Lipoamide acyltransferase component of branched-chain alpha-keto acid dehydrogenase complex, mitochondrial, DBT; Lipoprotein lipase; Liver and muscle phosphorylase kinase, PHKB; Liver phosphorylase kinase, PHKG2; Lysosomal acid lipase/cholesteryl ester hydrolase; Lysosomal alpha-glucosidase; Lysosomal alpha-mannosidase; Lysosomal protective protein; CLN6 Transmembrane ER Protein, CLN6; CLN8 Transmembrane ER And ERGIC Protein, CLN8; Lysosomal transmembrane CLN3 protein, CLN3; Lysosomal transmembrane CLN5 protein, CLN5; Lysosome-associated membrane glycoprotein 2; Lysosomal trafficking regulator, LYST; Malonyl-CoA decarboxylase, MLYCD; Matrilin-3; Matrix Gla protein; Melanophilin, MLPH; Methionine synthase reductase, MTRR; Methylene tetrahydrofolate homocysteine methyltransferase, MTR; Methylenetetrahydrofolate reductase, MTHFR; Methylmalonic semialdehyde dehydrogenase, ALDH6A1; Methylmalonyl-CoA mutase; Mevalonate kinase; Mitochondrial branched-chain aminotransferase 2, BCAT2; Mitochondrial ornithine translocase, SLC25A15; Methylmalonic aciduria type A, MMAA; Molybdopterin synthase, Gephyrin, MOCS1A; Mucolipin-1, MCOLN1; Muscle phosphorylase kinase, PHKA1; Myosin Va, MYO5A; Myosin light chain 4; N-Acetylgalactosamine-6 Sulfatase, GALNS; N-acetylglucosamine-6-sulfatase; Nicotinamide N-methyltransferase; NPC intracellular cholesterol transporter 1, NPC1; Palmitoyl-protein thioesterase-1, PPT1; Palmitoyl-protein thioesterase, PPT2; Pentraxin-related protein, PTX3; Peptidyl-prolyl cis-trans isomerase, FKBP10; Peroxidasin homolog; Peroxin-1, 2, 3, 5, 6, 7, 10, 12, 13, 14, 26, Phosphoacetylglucosamine mutase; Phosphoglucomutase-1; Phosphoglycerate kinase 1; Phosphoglycerate mutase 1; Pigment epithelium-derived factor, PEDF; Plasma alpha-L-fucosidase; Plasma membrane carnitine transport, OCTN2; Plasma protease C1 inhibitor; Plasminogen activator inhibitor 1; Procollagen-lysine,2-oxoglutarate 5-dioxygenase 1; Propionyl-CoA carboxylase; Prosaposin; Proteoglycan 4; Proteoglycan 4 C-terminal part; Pyruvate carboxylase; Pyruvate dehydrogenase complex, DLAT; Pyruvate dehydrogenase complex, PDHB; Pyruvate dehydrogenase complex, PDHX; Pyruvate dehydrogenase complex, PDP1; Ras-related protein Rab-27A, RAB27A; Retinol-binding protein 4; Ribonuclease T2; Semaphorin-7A; Sepiapterin reductase; Serine protease, HTRA1; Serotransferrin; Serpin B6; Serum amyloid A-1 protein; Short branched-chain acyl-CoA dehydrogenase, ACADSB; Sialic acid synthase; Sialidase-1; Sialin (sialic acid transport), SLC17A5; Solute Carrier Family 22 Member 5, SLC22A5; SPARC-related modular calcium-binding protein 2; Spectrin alpha chain, non-erythrocytic 1; Sphingomyelin phosphodiesterase, SMPD1; Succinyl-CoA 3-oxoacid-CoA transferase, OXCT1; Sushi repeat-containing protein, SRPX2; Tafazzin; Tenascin; Thrombospondin-2; Transforming growth factor-beta-induced protein ig-h3; Transitional endoplasmic reticulum ATPase; Triosephosphate isomerase; Tripeptidyl-peptidase 1; Tumor necrosis factor receptor superfamily member 11B; Vascular endothelial growth factor C; Versican core protein; Vimentin; Vitamin K-dependent protein S; X-linked phosphorylase kinase, PHKA2; Xaa-Pro dipeptidase; α-Fucosidase, FUCA1; α-Galactosidase A, GLA; α-N-Acetylglucosaminidase, NAGA; β-Glucocerebrosidase (aka Glucosylceramidase); GBA, β-glucuronidase, GUSB; β-mannosidasen; VEGFA; VEGF165; FGF2; FGF4; PDGF-BB (platelet-derived growth factor); Ang1 (angiopoiten 1), TGFβ (transforming growth factor); LPA-producing enzyme (AXT); and phthalimide neovascularization factor (PNF1).

37. The composition of claim 1, wherein the adipogenic cells comprise a heterologous nucleic acid.

38.-55. (canceled)

56. A syringe comprising the composition of claim 1.

57. (canceled)

58. (canceled)

59. A method for treating, preventing, or ameliorating a disease or disorder in a subject in need thereof, comprising administering a composition of claim 1 to the subject.

60.-65. (canceled)

66. The method of claim 59, wherein the subject has, is suspected of having, or is suspected of having an elevated risk for a disease or disorder selected from Lysosomal storage disorders, Metabolic disorders, Complement deficiencies, Adipocyte disorders, Endocrine disorders, Vascular diseases, Branched-chain amino acid metabolism disorders, Connective tissue disorders, Fatty acid transport and mitochrondrial oxidation disorders, Genetic dyslipidemias, Hematological disorders, Phenylalanine and tyrosine metabolism disorders, Purine metabolism disorders, Urea cycle disorders, Beta-amino acid and gamma-amino acid disorders, Ketone metabolism disorders, Galactosemia, Glycerol Metabolism Disorders, Glycine Metabolism Disorders, Lysine Metabolism Disorders, Methionine and Sulfur Metabolism Disorders, Peroxisome biogenesis and very long chain fatty acid metabolism disorders, Lysosomal storage disorders, Metabolic disorders, Hematological disorders, Bone and connective tissue disorders, Endocrine disorders, Inflammatory disorders, Monogenic disorders, Cancer, Cardiovascular disorders, Branched-chain amino acid metabolism disorders, Fatty acid transport and mitochrondrial oxidation disorders, Genetic dyslipidemias, Phenylalanine and tyrosine metabolism disorders, Purine metabolism disorders, Urea cycle disorders, Ketone metabolism disorders, Glycine Metabolism Disorders, Lysine Metabolism Disorders, Methionine and Sulfur Metabolism Disorders, Peroxisome biogenesis and very long chain fatty acid metabolism disorders, other protein deficiency disorders, Wolman disease, Obesity, C3 deficiency, Familial lipodystrophy, Cachexia, Hereditary angioedema, Propionic acidemia Type 1, Ehlers-Danlos syndrome, long-chain 3-hydroxy acyl-CoA dehydrogenase deficiency, Familial LPL deficiency, Protein S deficiency, Tyrosinemia type I, Adenine phosphoribosyltransferase deficiency, Citrullinemia type I, Methylmalonic semialdehyde dehydrogenase deficiency, Succinyl-CoA 3-oxoacid-CoA transferase deficiency, Galactose-1-phosphate uridyl transferase deficiency, Glycerol kinase deficiency, Nonketotic hyperglycinemia, Glutaric acidemia type I, Molybdenum cofactor defect, Zellweger syndrome, Cystinosis, T2D, Hemophilia A or B, Stickler syndrome, Osteoporosis, Rheumatoid Arthritis, A1AT deficiency, Breast cancer, Atherosclerosis, Isobutyryl-CoA dehydroqenase deficiency, carnitine-acylcarnitine translocase deficiency, Sitosterolemia, Phenylketonuria, Hereditary xanthinuria, Ornithine-transcarbamoylase deficiency, 3-Hydroxy-3-methylglutaryl-CoA synthase deficiency, Nonketotic hyperglycinemia, Hyperlysinemia, Homocystinuria, Refsum disease, or growth failure in children with kidney disease.

67.-70. (canceled)

71. The method of claim 59, wherein the composition comprises adipogenic cells that are transformed, comprising a heterologous nucleic acid comprising a therapeutic transgene, wherein the adipogenic cells comprise one or more of a gene, or genes associated with cystinosin, GLP-1, Factor VIII, Factor IX, COL2A1, Parathyroid hormone (1-84), alkaline phosphatase, alpha-1 antitrypsin, Trastuzumab, Apolipoprotein A1, Isobutyryl-CoA dehydrogenase, SLC25A20, ATP-binding cassette sub-family G member 5, ABCG5, Phenylalanine hydroxylase, Xanthine dehydrogenase, Ornithine-transcarbamoylase, 3-Hydroxy-3-methylglutaryl-CoA synthase, Glycine cleavage system P protein, Lysine:α-ketoglutarate reductase, Cystathionine β-synthase, Phytanoyl-CoA hydroxylase, and human growth hormone (somatotropin), wherein the gene is in operative association with an adipocyte-specific promoter.

72.-75. (canceled)

76. A process for in vivo electroporation of adipogenic cells comprising:
injecting the adipogenic cells into adipose tissue of a subject;
placing the adipose tissue between a first plate electrode and a second plate electrode; and
passing a current from the first plate electrode through the adipose tissue to the second plate electrode.

77.-82. (canceled)

83. An allogenic, non-immunogenic, long-acting composition comprising a therapeutically effective amount of a substantially pure adipogenic cells, wherein the adipogenic cells are obtainable from ASCs that express elevated levels of CD10 compared to wild type ASCs and/or unenriched ASCs.

84.-87. (canceled)


SEQUENCE LISTING (35 sequences)

SEQ ID NO: 1 - DNA, 720 bp, Artificial Sequence
  Synthetic Sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - DNA, 1653 bp, Artificial Sequence
  Synthetic Sequence
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 3 - DNA, 589 bp, Artificial Sequence
  Synthetic Sequence
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 4 - DNA, 2741 bp, Artificial Sequence
  Synthetic Sequence
ctctttccacatgacggcctttgtggtgggtggcagattgccctgaggcctcgcaaaatgctaggctttcacaatgtcactgactgacagccaggcccagcacagtcttggtgtgattgtggggctaaagttattccaccttgtgcaatagctacagctttctctaaccagctgcattcttataaagttagaagaaaatacttttttttttttgagatggattctcgctctgttgcccaggctggagtgcaatggtgcgatctcggctcgctgcaacctccgcctcctgggttcaaacgattctcctccctcagacccccgagtagctgggattgcaggtgcctgccaccacgcccggctaacttttttgtatttttagtggagacggggtttcaccatcttcgtcaggctggtctcagactcctgacctcaagtgatctgcccgcctcagcctcccaaaatgctgggattacaggcatgagctactgtgcccggccaaagaaaatactttttatgccagccctgaaactaccctgaagcacatacatcaaccttgaggcctcacactccatcaagaggggtgaagggcatgaggaattagaaagcatagggatttttagttagacagatctggttcaaatcctagacttgtgccttgaacaaattatttaccctcattgaactctagattcattatttgtaaaatgaaagacaataatagttatctccaaaggaaagttgaatatgatcattcatttattcattaattcaacatttattattgcctactttgtgccaggttctattctaggaactaagggatacaactttgaataggcaaaatctctgctctcctgaagtttacttttttttttttttttgagacagagtttcactcttgtcacccaggctagagcgcaatggtgctcttggctcactgcaacctccacctcctgggttcaagtgattctcttgtctcagcctcccaagtagctgggactacaggtatgtgccaccacgcccggctatttctgcatttttagtagagatggggtttcaccatgttggccagactggtctcaaactcctgatctcaggtgatatgcctgtcttggccttccaaagtactgggattacaggcctgagccactgcacctgacctgaagtttatgttctattaaatagcaacagacagtaacataaaccaaaaataaataggaaaacaccataacaaaaatcaaacagtgatataattgagagttgcttctatttctttttgttgtcttcttggttcaatcagcctgctaaactatatggaacctcattttcatgggccacttatttaagccgggggaccttggaaagtctctcatgtctctcatctcaacggcctaatgtgacttctcttgaaatatttggacattagcaggaagctgaggctttacatcagatctttactttaatggtggacttgactttactggtagatttttaggctctgtgtggactgtggagatgatatctggggggcaggcagacacttgccctgcctctgtctgagaaaattctgttttggatgtcttgttgaagttggtgctggcatcctaagcccttgctggggtcgtaatttaattcatcagaatgtgtggcttgcaagaaccggctcagatcctgcccttcaaaaacaaaacatgagcgtgccaagaaagtccaaggtgttgaatgttgccacttcaagcctaaactttctaggaacacctaagtgggtggcagcttccagttctccaggctgcttctaggccagagctgggttccacaagagacagaataggcatatatatgcttaaggaactggaaaaacaggctctctctctctcacaaacacacacacacacataccaaggtagctgtcaaaatgttatccgaaattttggaaccaaaaaatcttgaaagatggtattccaatatcacattttatgtaagttttctattatattagattcaaattacgattcgaggccacaagctttaagaattcagggcctttttaacttgccaagccccacaccactccaggaacttccccacaccccagttctcagaattcatgtgcaaggtctttcctaaatccagggtccaggtcagagagtggaggatgtgctctatttcttacctgattgcagacccctctgacagtgctcccttctgaagcactcactgtctgaacgtacacagtctcagacttaatcatgcacagtgagcaagactgtggtgtgataattggcgtccctgacttattagggcaaatctatgggagggggagacctcctggaccactgagcaattaattcatttacattaggaagtttctccgtcagatgcaggaaaaaaatcttgttttcctgctgtggttttgacttttgccccatcttctgttgctgttgtaggaggcaaaataagggtcaaggcctggaaacacaagtgctttgactgaagctccacttggcttccgaagcccaagctgggttgtaccaggttccctagggtgcaggctgtgggcaactgccagggacatgtgcctgcccaccggcctctggccctcactgagttggccaatgggaaatgacaattgtgaggtggggactgcctgcccccgtgagtaccaggctgttgaggctgggccatctcctcctcacttccattctgactgcagtctgtggttctgattccataccagaggg

SEQ ID NO: 5 - DNA, 2266 bp, Artificial Sequence
  Synthetic Sequence
aaaccacccagcaaaaaaccaaaccgcctagcctcaagacatgtgtggttgaatgtttttcacttctagtcgctaagcaagtgtgtgtttttacacaatgccctctgtggtgagtggcggattcccctgagagttcaccaaatgataggctttcacaatgctcccgggtgtctaccagacccagcaaagtattgatgtggttttggggtgaaagtcactctgtcttgtgcaatagttagaatctgctgaaaccagcagtgttcctatatgggacaggggtccagagctaacccggaggctataactgagcagaggtgaagaccacgaggcattggggagcgtatgccctttgtggtcagagagatctagcttcgtgccttgggtctgtgtctctccctcttactggcttctggcttcttcattaagtgggagacaaccacaggtatctgtatgggaagactcgactaccccttgactcaacattgcttgttacttactttgtacaagatactacttagtctaggggttatggagcataacctcaagtaggtaaagcccctgctccagcgtgtttgcattccagtaagaagcgaaagacagtaacacacatacaaaataagtaagaaaatgcaacaacagcaacaacaacaacacacacacacaaagtaagcaaaacgctaagggaaagatagagagtgatacagctttgagttgctgtagttcttctctctcctttgcttcatacagtttgcttgggaagtgtccagggccatggggtcacaactaacagcccttggaaatgagcttgtgtccttaatcttcatgacctaacgtgatttctctagaaacatcagtgcattaacaggaagacaagatggaagatcatattttggctctccttccttggtgggttgacactgctggtcctatccactagtaaaagcatgactcttaggctctgtgtggccagtggaaggtggcagttggaggaagcagatgcttggccagcctttgcctgggagcagtctagctctgagtgtcttattggagcagctgctggcatccagagttctttttggattcacgatttaattcaaaagctttgtgctcccgagaatcagctctggtctttcaaaaataagatgtgagtccgccgagaggctcccaaggtattgccttgccaactgcaagccttttaggagcagtttagtgagtggtgactgctagttgcagttggctgttagcccagagctaataatagatagaaaaggtatatacttaaggagtctggaaactgaggtttatctactcacagaaaatgagtttctaaaaaactagcttgaaacttacccagaaaaatcttagaacatggttctccaatgtcaaggtaagtgttctgtgacactgggcttgaattatgtagggaccacagattttagaatttggacccctgaacttgcttcacaccccaccaggaaccttcctgtacaacagccctcagaattcatctacatggtcttttctcagtatgggatccggtctagcaagtggagcacaccttctattgcttaaagatttgtttatgtatatgggtattttggctgcatgcatatttgcacaccaaaagaaggcagcggatcccatggaattactgtgggtgctgggaattgaactcaggacctctggaagaatagccagtgctcttaaccactgagccatgcctgcagtccatctattttttattctagtacagcccctcttcattcttactgaaatagtaatgcctgaaccacacagcttcacatttagttacaaagaaagagtgggagtatcatgtgacaattagtgttgttgactctccaggacaaacttatgggaaagggaggtctcctgacccctgaacaatcattttacttgaggataattttcattgcactcagaaacatgctgaattattgtccttacccttgccccatctcttgctctggtagagaatggccaaagcctggaaacaggatggcttgacagaagctctacttggcttcccagacccaagctggattaaaccaggttccctaaggagtcttaaggcagctgccaggagcaaggggcccactcattggctattggccttgactgggttggccaatggtaagctggggtctgcctgtccccatgagtaccagactaatgagacctggccactttctcctcatttctgtctgtacgattgtcagtggatctgacgacaccaaaaggtaagaac

SEQ ID NO: 6 - DNA, 1026 bp, Artificial Sequence
  Synthetic Sequence
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 7 - DNA, 600 bp, Artificial Sequence
  Synthetic Sequence
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 8 - DNA, 558 bp, Artificial Sequence
  Synthetic Sequence
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 9 - DNA, 582 bp, Artificial Sequence
  Synthetic Sequence
atgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagaacatcaccaccggctgcgccgagcactgcagcctgaacgagaacatcaccgtgcccgacaccaaggtgaacttctacgcctggaagcggatggaggtgggccagcaggccgtggaggtgtggcagggcctggccctgctgagcgaggccgtgctgcggggccaggccctgctggtgaacagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccggaccggcgaccggtga

SEQ ID NO: 10 - DNA, 576 bp, Artificial Sequence
  Synthetic Sequence
atgggggtgcccgaacgtcccaccctgctgcttttactctccttgctactgattcctctgggcctcccagtcctctgtgctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgctccgggtgctgggagctcaggaattgatgtcacctccagataccaccccacctgctccactccgaacactcacagtggatactttctgcaagctcttccgggtctacgccaacttcctccgggggaaactgaagctgtacacgggagaggtctgcaggagaggggacaggtga

SEQ ID NO: 11 - DNA, 1359 bp, Artificial Sequence
  Synthetic Sequence
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaa

SEQ ID NO: 12 - DNA, 1362 bp, Artificial Sequence
  Synthetic Sequence
atggcagctgttgtcctggagaacggagtcctgagcagaaaactctcagactttgggcaggaaacaagttacatcgaagacaactccaatcaaaatggtgctgtatctctgatattctcactcaaagaggaagttggtgccctggccaaggtcctgcgcttatttgaggagaatgagatcaacctgacacacattgaatccagaccttctcgtttaaacaaagatgagtatgagtttttcacctatctggataagcgtagcaagcccgtcctgggcagcatcatcaagagcctgaggaacgacattggtgccactgtccatgagctttcccgagacaaggaaaagaacacagtgccctggttcccaaggaccattcaggagctggacagattcgccaatcagattctcagctatggagccgaactggatgcagaccacccaggctttaaagatcctgtgtaccgggcgagacgaaagcagtttgctgacattgcctacaactaccgccatgggcagcccattcctcgggtggaatacacagaggaggagaggaagacctggggaacggtgttcaggactctgaaggccttgtataaaacacatgcctgctacgagcacaaccacatcttccctcttctggaaaagtactgcggtttccgtgaagacaacatcccgcagctggaagatgtttctcagtttctgcagacttgtactggtttccgcctccgtcctgttgctggcttactgtcgtctcgagatttcttgggtggcctggccttccgagtcttccactgcacacagtacattaggcatggatctaagcccatgtacacacctgaacctgatatctgtcatgaactcttgggacatgtgcccttgttttcagatagaagctttgcccagttttctcaggaaattgggcttgcatcgctgggggcacctgatgagtacattgagaaactggccacaatttactggtttactgtggagtttgggctttgcaaggaaggagattctataaaggcatatggtgctgggctcttgtcatcctttggagaattacagtactgtttatcagacaagccaaagctcctgcccctggagctagagaagacagcctgccaggagtatactgtcacagagttccagcccctgtactatgtggccgagagtttcaatgatgccaaggagaaagtgaggacttttgctgccacaatcccccggcccttctccgttcgctatgacccctacactcaaagggttgaggtcctggacaatactcagcagttgaagattttagctgactccattaatagtgaggttggaatcctttgccatgccctgcagaaaataaagtcatga

SEQ ID NO: 13 - DNA, 587 bp, Artificial Sequence
  Synthetic Sequence
ggatgaactgctccgccctctgtctctttggcagggttggagcccactgtggcctgagcgacttctatggctccctttctgtgattttcatggtttctgagctcttttcccccgctttatgattttctctttttgtctctctcttgctaaacctccttcgtatatgccctctcaggtttcatttctgaatcatctactgtgaactattcccattgtttgccagaagccccctggttcttccttctagaaggaataatggggggaagttcaatgcattagcttttgacagtcaaaacaggaacctttaaaatactctgttcatggttaaaaataatttgtactctaagtccagtgatcattgccagggagaaccaaagttgagaaatttctattaaaaacatgactcagaggaaaacatacagggtctggtcatgaaggaaatgatctggcccccattggtcactcctacagtcacatggtcagggcatctttaaaagtgagctatctggacttcagaggctcatagcaccctcctgtgctgcagcctttctcacctggaagacagctcctcctcgaaggtttacaaa

SEQ ID NO: 14 - protein, 15 aa, Artificial Sequence
  Synthetic Sequence
GLNDIFEAQKIEWHE

SEQ ID NO: 15 - protein, 26 aa, Artificial Sequence
  Synthetic Sequence
KRRWKKNFIAVSAANRFKKISSSGAL

SEQ ID NO: 16 - protein, 6 aa, Artificial Sequence
  Synthetic Sequence
EEEEEE

SEQ ID NO: 17 - protein, 13 aa, Artificial Sequence
  Synthetic Sequence
GAPVPYPDPLEPR

SEQ ID NO: 18 - protein, 8 aa, Artificial Sequence
  Synthetic Sequence
DYKDDDDK

SEQ ID NO: 19 - protein, 9 aa, Artificial Sequence
  Synthetic Sequence
YPYDVPDYA

SEQ ID NO: 20 - protein, 6 aa, Artificial Sequence
  Synthetic Sequence
HHHHHH

SEQ ID NO: 21 - protein, 10 aa, Artificial Sequence
  Synthetic Sequence
EQKLISEEDL

SEQ ID NO: 22 - protein, 18 aa, Artificial Sequence
  Synthetic Sequence
TKENPRSNQEESYDDNES

SEQ ID NO: 23 - protein, 15 aa, Artificial Sequence
  Synthetic Sequence
KETAAAKFERQHMDS

SEQ ID NO: 24 - protein, 38 aa, Artificial Sequence
  Synthetic Sequence
MDEKTTGWRGGHVVEGLAGELEQLRARLEHHPQGQREP

SEQ ID NO: 25 - protein, 13 aa, Artificial Sequence
  Synthetic Sequence
SLAELLNAGLGGS

SEQ ID NO: 26 - protein, 8 aa, Artificial Sequence
  Synthetic Sequence
TQDPSRVG

SEQ ID NO: 27 - protein, 12 aa, Artificial Sequence
  Synthetic Sequence
PDRVRAVSHWSS

SEQ ID NO: 28 - protein, 8 aa, Artificial Sequence
  Synthetic Sequence
WSHPQFEK

SEQ ID NO: 29 - protein, 6 aa, Artificial Sequence
  Synthetic Sequence
CCPGCC

SEQ ID NO: 30 - protein, 10 aa, Artificial Sequence
  Synthetic Sequence
EVHTNQDPLD

SEQ ID NO: 31 - protein, 14 aa, Artificial Sequence
  Synthetic Sequence
GKPIPNPLLGLDST

SEQ ID NO: 32 - protein, 11 aa, Artificial Sequence
  Synthetic Sequence
YTDIEMNRLGK

SEQ ID NO: 33 - protein, 8 aa, Artificial Sequence
  Synthetic Sequence
DLYDDDDK

SEQ ID NO: 34 - DNA, 19 bp, Artificial Sequence
  Synthetic Sequence
gacaccatcgaccacgacc

SEQ ID NO: 35 - DNA, 19 bp, Artificial Sequence
  Synthetic Sequence
gccacgtcgcagagagttc